SEQUENCE LISTING (404 sequences)

SEQ ID NO: 1 - RNA, 20 nt, synthetic construct
  source 1..20: mol_type = other RNA
gctagatcaagaagtacagg

SEQ ID NO: 2 - RNA, 20 nt, synthetic construct
  source 1..20: mol_type = other RNA
gcaagtgcaggagtcagtga

SEQ ID NO: 3 - RNA, 20 nt, synthetic construct
  source 1..20: mol_type = other RNA
agaagtacaggaggagactc

SEQ ID NO: 4 - RNA, 20 nt, synthetic construct
  source 1..20: mol_type = other RNA
gaagtacaggaggagactca

SEQ ID NO: 5 - RNA, 20 nt, synthetic construct
  source 1..20: mol_type = other RNA
acctgtcaggtgaagttcgc

SEQ ID NO: 6 - RNA, 20 nt, synthetic construct
  source 1..20: mol_type = other RNA
gttccacagaacccaacaac

SEQ ID NO: 7 - RNA, 20 nt, synthetic construct
  source 1..20: mol_type = other RNA
cactcacctgcccacagcag

SEQ ID NO: 8 - RNA, 20 nt, synthetic construct
  source 1..20: mol_type = other RNA
gaaccagtaaccatgaactg

SEQ ID NO: 9 - RNA, 20 nt, synthetic construct
  source 1..20: mol_type = other RNA
cggaaccagtaaccatgaac

SEQ ID NO: 10 - RNA, 20 nt, synthetic construct
  source 1..20: mol_type = other RNA
acaggcccaggacacagagc

SEQ ID NO: 11 - RNA, 20 nt, synthetic construct
  source 1..20: mol_type = other RNA
gacaaccaggagaagatcgg

SEQ ID NO: 12 - RNA, 20 nt, synthetic construct
  source 1..20: mol_type = other RNA
ccactcacctgcccacagca

SEQ ID NO: 13 - RNA, 20 nt, synthetic construct
  source 1..20: mol_type = other RNA
caagtctagtgaggagaaag

SEQ ID NO: 14 - RNA, 20 nt, synthetic construct
  source 1..20: mol_type = other RNA
acttacaggtgacgttgagc

SEQ ID NO: 15 - RNA, 20 nt, synthetic construct
  source 1..20: mol_type = other RNA
aacatctaggagaggaagag

SEQ ID NO: 16 - RNA, 20 nt, synthetic construct
  source 1..20: mol_type = other RNA
ttcctacctgagccatctcc

SEQ ID NO: 17 - RNA, 20 nt, synthetic construct
  source 1..20: mol_type = other RNA
atgctcacatgaagaagatg

SEQ ID NO: 18 - RNA, 20 nt, synthetic construct
  source 1..20: mol_type = other RNA
cactctgatgggagacacca

SEQ ID NO: 19 - RNA, 20 nt, synthetic construct
  source 1..20: mol_type = other RNA
tgtctcctgcaaacgaagga

SEQ ID NO: 20 - RNA, 20 nt, synthetic construct
  source 1..20: mol_type = other RNA
ttctcaaagttcccacatcc

SEQ ID NO: 21 - RNA, 20 nt, synthetic construct
  source 1..20: mol_type = other RNA
atgctcagggaacacgtatc

SEQ ID NO: 22 - RNA, 20 nt, synthetic construct
  source 1..20: mol_type = other RNA
gatgctcagggaacacgtat

SEQ ID NO: 23 - RNA, 20 nt, synthetic construct
  source 1..20: mol_type = other RNA
tcacagattggtgagtagcc

SEQ ID NO: 24 - RNA, 20 nt, synthetic construct
  source 1..20: mol_type = other RNA
gacatctctcgactctccag

SEQ ID NO: 25 - RNA, 20 nt, synthetic construct
  source 1..20: mol_type = other RNA
tgagccaaaggaggaccatc

SEQ ID NO: 26 - RNA, 20 nt, synthetic construct
  source 1..20: mol_type = other RNA
cctgcccaaggcttcccacc

SEQ ID NO: 27 - RNA, 20 nt, synthetic construct
  source 1..20: mol_type = other RNA
tcttaccttccttcgtttgc

SEQ ID NO: 28 - RNA, 20 nt, synthetic construct
  source 1..20: mol_type = other RNA
tttggatctaaaacggtgac

SEQ ID NO: 29 - RNA, 20 nt, synthetic construct
  source 1..20: mol_type = other RNA
ctcacctgttctgtgattac

SEQ ID NO: 30 - RNA, 20 nt, synthetic construct
  source 1..20: mol_type = other RNA
ggtcgcactctaggggtaaa

SEQ ID NO: 31 - RNA, 20 nt, synthetic construct
  source 1..20: mol_type = other RNA
tacctcggaggaaagagaaa

SEQ ID NO: 32 - RNA, 20 nt, synthetic construct
  source 1..20: mol_type = other RNA
tggagaaaaccatgaaactg

SEQ ID NO: 33 - RNA, 20 nt, synthetic construct
  source 1..20: mol_type = other RNA
cttacctgggacgaaaagaa

SEQ ID NO: 34 - RNA, 20 nt, synthetic construct
  source 1..20: mol_type = other RNA
caatcgctggaggaaggaaa

SEQ ID NO: 35 - RNA, 20 nt, synthetic construct
  source 1..20: mol_type = other RNA
cacatcaaggagtaattacc

SEQ ID NO: 36 - RNA, 20 nt, synthetic construct
  source 1..20: mol_type = other RNA
gactcgcagacgccctctgc

SEQ ID NO: 37 - RNA, 20 nt, synthetic construct
  source 1..20: mol_type = other RNA
tgctgctcagggaccactga

SEQ ID NO: 38 - RNA, 20 nt, synthetic construct
  source 1..20: mol_type = other RNA
tgtgctcagtactggggaga

SEQ ID NO: 39 - RNA, 20 nt, synthetic construct
  source 1..20: mol_type = other RNA
ccccagaagaattcctctga

SEQ ID NO: 40 - RNA, 20 nt, synthetic construct
  source 1..20: mol_type = other RNA
ctcgatgtgaagaaggaaac

SEQ ID NO: 41 - RNA, 20 nt, synthetic construct
  source 1..20: mol_type = other RNA
aaggcgacagagatgcctga

SEQ ID NO: 42 - RNA, 20 nt, synthetic construct
  source 1..20: mol_type = other RNA
gtcatgttccagacctggct

SEQ ID NO: 43 - RNA, 20 nt, synthetic construct
  source 1..20: mol_type = other RNA
ccacactccagtttgtatat

SEQ ID NO: 44 - RNA, 20 nt, synthetic construct
  source 1..20: mol_type = other RNA
tcctgttaattaatggaaaa

SEQ ID NO: 45 - RNA, 20 nt, synthetic construct
  source 1..20: mol_type = other RNA
tgggacatctgcaatcagaa

SEQ ID NO: 46 - RNA, 20 nt, synthetic construct
  source 1..20: mol_type = other RNA
ggtaatatcacctattgttg

SEQ ID NO: 47 - RNA, 20 nt, synthetic construct
  source 1..20: mol_type = other RNA
ttaccacatgttggcttaga

SEQ ID NO: 48 - RNA, 20 nt, synthetic construct
  source 1..20: mol_type = other RNA
aacatggcaggagagtaaaa

SEQ ID NO: 49 - RNA, 20 nt, synthetic construct
  source 1..20: mol_type = other RNA
caaatccatctccttggaca

SEQ ID NO: 50 - RNA, 20 nt, synthetic construct
  source 1..20: mol_type = other RNA
ttatgccaaatccatctcct

SEQ ID NO: 51 - RNA, 20 nt, synthetic construct
  source 1..20: mol_type = other RNA
tatatccagggactctccag

SEQ ID NO: 52 - RNA, 20 nt, synthetic construct
  source 1..20: mol_type = other RNA
ggaaggagccattatatcca

SEQ ID NO: 53 - RNA, 20 nt, synthetic construct
  source 1..20: mol_type = other RNA
catggttactggttccggga

SEQ ID NO: 54 - RNA, 20 nt, synthetic construct
  source 1..20: mol_type = other RNA
agttcatggttactggttcc

SEQ ID NO: 55 - RNA, 20 nt, synthetic construct
  source 1..20: mol_type = other RNA
actccccagttcatggttac

SEQ ID NO: 56 - RNA, 20 nt, synthetic construct
  source 1..20: mol_type = other RNA
gacaagaactccccagttca

SEQ ID NO: 57 - RNA, 10 nt, synthetic construct
  source 1..10: mol_type = other RNA
gactctccag

SEQ ID NO: 58 - RNA, 10 nt, synthetic construct
  source 1..10: mol_type = other RNA
attatatcca

SEQ ID NO: 59 - RNA, 10 nt, synthetic construct
  source 1..10: mol_type = other RNA
ggttccggga

SEQ ID NO: 60 - RNA, 10 nt, synthetic construct
  source 1..10: mol_type = other RNA
tactggttcc

SEQ ID NO: 61 - RNA, 10 nt, synthetic construct
  source 1..10: mol_type = other RNA
tcatggttac

SEQ ID NO: 62 - RNA, 10 nt, synthetic construct
  source 1..10: mol_type = other RNA
ccccagttca

SEQ ID NO: 63 - RNA, 20 nt, synthetic construct
  source 1..20: mol_type = other RNA
ccccacaggggtcctggcta

SEQ ID NO: 64 - RNA, 20 nt, synthetic construct
  source 1..20: mol_type = other RNA
tcgtttccccacaggggtcc

SEQ ID NO: 65 - RNA, 20 nt, synthetic construct
  source 1..20: mol_type = other RNA
tgaccctcgtttccccacag

SEQ ID NO: 66 - RNA, 20 nt, synthetic construct
  source 1..20: mol_type = other RNA
gctgaccctcgtttccccac

SEQ ID NO: 67 - RNA, 20 nt, synthetic construct
  source 1..20: mol_type = other RNA
gggagaggggttgtcgggct

SEQ ID NO: 68 - RNA, 20 nt, synthetic construct
  source 1..20: mol_type = other RNA
ctgtggggagaggggttgtc

SEQ ID NO: 69 - RNA, 10 nt, synthetic construct
  source 1..10: mol_type = other RNA
gtcctggcta

SEQ ID NO: 70 - RNA, 10 nt, synthetic construct
  source 1..10: mol_type = other RNA
acaggggtcc

SEQ ID NO: 71 - RNA, 10 nt, synthetic construct
  source 1..10: mol_type = other RNA
ttccccacag

SEQ ID NO: 72 - RNA, 10 nt, synthetic construct
  source 1..10: mol_type = other RNA
gtttccccac

SEQ ID NO: 73 - RNA, 10 nt, synthetic construct
  source 1..10: mol_type = other RNA
ttgtcgggct

SEQ ID NO: 74 - RNA, 10 nt, synthetic construct
  source 1..10: mol_type = other RNA
aggggttgtc

SEQ ID NO: 75 - RNA, 20 nt, synthetic construct
  source 1..20: mol_type = other RNA
tatatccagggactctccag

SEQ ID NO: 76 - RNA, 20 nt, synthetic construct
  source 1..20: mol_type = other RNA
ggaaggagccattatatcca

SEQ ID NO: 77 - RNA, 20 nt, synthetic construct
  source 1..20: mol_type = other RNA
catggttactggttccggga

SEQ ID NO: 78 - RNA, 20 nt, synthetic construct
  source 1..20: mol_type = other RNA
agttcatggttactggttcc

SEQ ID NO: 79 - RNA, 20 nt, synthetic construct
  source 1..20: mol_type = other RNA
actccccagttcatggttac

SEQ ID NO: 80 - RNA, 20 nt, synthetic construct
  source 1..20: mol_type = other RNA
gacaagaactccccagttca

SEQ ID NO: 81 - RNA, 10 nt, synthetic construct
  source 1..10: mol_type = other RNA
gactctccag

SEQ ID NO: 82 - RNA, 10 nt, synthetic construct
  source 1..10: mol_type = other RNA
attatatcca

SEQ ID NO: 83 - RNA, 10 nt, synthetic construct
  source 1..10: mol_type = other RNA
ggttccggga

SEQ ID NO: 84 - RNA, 10 nt, synthetic construct
  source 1..10: mol_type = other RNA
tactggttcc

SEQ ID NO: 85 - RNA, 10 nt, synthetic construct
  source 1..10: mol_type = other RNA
tcatggttac

SEQ ID NO: 86 - RNA, 10 nt, synthetic construct
  source 1..10: mol_type = other RNA
ccccagttca

SEQ ID NO: 87 - RNA, 20 nt, synthetic construct
  source 1..20: mol_type = other RNA
tatatccagggactctccag

SEQ ID NO: 88 - RNA, 20 nt, synthetic construct
  source 1..20: mol_type = other RNA
ggaaggagccattatatcca

SEQ ID NO: 89 - RNA, 20 nt, synthetic construct
  source 1..20: mol_type = other RNA
catggttactggttccggga

SEQ ID NO: 90 - RNA, 20 nt, synthetic construct
  source 1..20: mol_type = other RNA
agttcatggttactggttcc

SEQ ID NO: 91 - RNA, 20 nt, synthetic construct
  source 1..20: mol_type = other RNA
actccccagttcatggttac

SEQ ID NO: 92 - RNA, 20 nt, synthetic construct
  source 1..20: mol_type = other RNA
gacaagaactccccagttca

SEQ ID NO: 93 - RNA, 10 nt, synthetic construct
  source 1..10: mol_type = other RNA
gactctccag

SEQ ID NO: 94 - RNA, 10 nt, synthetic construct
  source 1..10: mol_type = other RNA
attatatcca

SEQ ID NO: 95 - RNA, 10 nt, synthetic construct
  source 1..10: mol_type = other RNA
ggttccggga

SEQ ID NO: 96 - RNA, 10 nt, synthetic construct
  source 1..10: mol_type = other RNA
tactggttcc

SEQ ID NO: 97 - RNA, 10 nt, synthetic construct
  source 1..10: mol_type = other RNA
tcatggttac

SEQ ID NO: 98 - RNA, 10 nt, synthetic construct
  source 1..10: mol_type = other RNA
ccccagttca

SEQ ID NO: 99 - RNA, 20 nt, synthetic construct
  source 1..20: mol_type = other RNA
accccacggccccaggacca

SEQ ID NO: 100 - RNA, 20 nt, synthetic construct
  source 1..20: mol_type = other RNA
cataatcaccccacggcccc

SEQ ID NO: 101 - RNA, 20 nt, synthetic construct
  source 1..20: mol_type = other RNA
cggtgctcataatcacccca

SEQ ID NO: 102 - RNA, 20 nt, synthetic construct
  source 1..20: mol_type = other RNA
ccccaggactactcactcct

SEQ ID NO: 103 - RNA, 20 nt, synthetic construct
  source 1..20: mol_type = other RNA
cccccacctccctgggcccc

SEQ ID NO: 104 - RNA, 10 nt, synthetic construct
  source 1..10: mol_type = other RNA
cccaggacca

SEQ ID NO: 105 - RNA, 10 nt, synthetic construct
  source 1..10: mol_type = other RNA
ccacggcccc

SEQ ID NO: 106 - RNA, 10 nt, synthetic construct
  source 1..10: mol_type = other RNA
aatcacccca

SEQ ID NO: 107 - RNA, 10 nt, synthetic construct
  source 1..10: mol_type = other RNA
actcactcct

SEQ ID NO: 108 - RNA, 10 nt, synthetic construct
  source 1..10: mol_type = other RNA
cctgggcccc

SEQ ID NO: 109 - RNA, 20 nt, synthetic construct
  source 1..20: mol_type = other RNA
tcatctctacccccaactga

SEQ ID NO: 110 - RNA, 20 nt, synthetic construct
  source 1..20: mol_type = other RNA
tggtttctggcaggagtaag

SEQ ID NO: 111 - RNA, 20 nt, synthetic construct
  source 1..20: mol_type = other RNA
tgtggtttctggcaggagta

SEQ ID NO: 112 - RNA, 10 nt, synthetic construct
  source 1..10: mol_type = other RNA
ccccaactga

SEQ ID NO: 113 - RNA, 10 nt, synthetic construct
  source 1..10: mol_type = other RNA
caggagtaag

SEQ ID NO: 114 - RNA, 10 nt, synthetic construct
  source 1..10: mol_type = other RNA
ggcaggagta

SEQ ID NO: 115 - RNA, 20 nt, synthetic construct
  source 1..20: mol_type = other RNA
tggggaaacgagggtcagct

SEQ ID NO: 116 - RNA, 20 nt, synthetic construct
  source 1..20: mol_type = other RNA
ggacccctgtggggaaacga

SEQ ID NO: 117 - RNA, 20 nt, synthetic construct
  source 1..20: mol_type = other RNA
ccatagccaggacccctgtg

SEQ ID NO: 118 - RNA, 20 nt, synthetic construct
  source 1..20: mol_type = other RNA
atccatagccaggacccctg

SEQ ID NO: 119 - RNA, 20 nt, synthetic construct
  source 1..20: mol_type = other RNA
agaaatttggatccatagcc

SEQ ID NO: 120 - RNA, 10 nt, synthetic construct
  source 1..10: mol_type = other RNA
agggtcagct

SEQ ID NO: 121 - RNA, 10 nt, synthetic construct
  source 1..10: mol_type = other RNA
ggggaaacga

SEQ ID NO: 122 - RNA, 10 nt, synthetic construct
  source 1..10: mol_type = other RNA
gacccctgtg

SEQ ID NO: 123 - RNA, 10 nt, synthetic construct
  source 1..10: mol_type = other RNA
aggacccctg

SEQ ID NO: 124 - RNA, 10 nt, synthetic construct
  source 1..10: mol_type = other RNA
atccatagcc

SEQ ID NO: 125 - RNA, 20 nt, synthetic construct
  source 1..20: mol_type = other RNA
tgtggccactggagagtccc

SEQ ID NO: 126 - RNA, 20 nt, synthetic construct
  source 1..20: mol_type = other RNA
tctagcttgtttgtggccac

SEQ ID NO: 127 - RNA, 20 nt, synthetic construct
  source 1..20: mol_type = other RNA
ttcttgatctagcttgtttg

SEQ ID NO: 128 - RNA, 10 nt, synthetic construct
  source 1..10: mol_type = other RNA
ggagagtccc

SEQ ID NO: 129 - RNA, 10 nt, synthetic construct
  source 1..10: mol_type = other RNA
ttgtggccac

SEQ ID NO: 130 - RNA, 10 nt, synthetic construct
  source 1..10: mol_type = other RNA
agcttgtttg

SEQ ID NO: 131 - RNA, 20 nt, synthetic construct
  source 1..20: mol_type = other RNA
tgtggccactggagagtccc

SEQ ID NO: 132 - RNA, 20 nt, synthetic construct
  source 1..20: mol_type = other RNA
tctagcttgtttgtggccac

SEQ ID NO: 133 - RNA, 20 nt, synthetic construct
  source 1..20: mol_type = other RNA
ttcttgatctagcttgtttg

SEQ ID NO: 134 - RNA, 10 nt, synthetic construct
  source 1..10: mol_type = other RNA
ggagagtccc

SEQ ID NO: 135 - RNA, 10 nt, synthetic construct
  source 1..10: mol_type = other RNA
ttgtggccac

SEQ ID NO: 136 - RNA, 10 nt, synthetic construct
  source 1..10: mol_type = other RNA
agcttgtttg

SEQ ID NO: 137 - RNA, 20 nt, synthetic construct
  source 1..20: mol_type = other RNA
gacaaccaggagaagatcgg

SEQ ID NO: 138 - RNA, 20 nt, synthetic construct
  source 1..20: mol_type = other RNA
gctgacaaccaggagaagat

SEQ ID NO: 139 - RNA, 20 nt, synthetic construct
  source 1..20: mol_type = other RNA
ggtgggggcagctgacaacc

SEQ ID NO: 140 - RNA, 20 nt, synthetic construct
  source 1..20: mol_type = other RNA
cctggggcccagggaggtgg

SEQ ID NO: 141 - RNA, 20 nt, synthetic construct
  source 1..20: mol_type = other RNA
agtcctggggcccagggagg

SEQ ID NO: 142 - RNA, 20 nt, synthetic construct
  source 1..20: mol_type = other RNA
agtagtcctggggcccaggg

SEQ ID NO: 143 - RNA, 20 nt, synthetic construct
  source 1..20: mol_type = other RNA
agtgagtagtcctggggccc

SEQ ID NO: 144 - RNA, 20 nt, synthetic construct
  source 1..20: mol_type = other RNA
ccgaggagtgagtagtcctg

SEQ ID NO: 145 - RNA, 20 nt, synthetic construct
  source 1..20: mol_type = other RNA
caccgaggagtgagtagtcc

SEQ ID NO: 146 - RNA, 10 nt, synthetic construct
  source 1..10: mol_type = other RNA
agaagatcgg

SEQ ID NO: 147 - RNA, 10 nt, synthetic construct
  source 1..10: mol_type = other RNA
aggagaagat

SEQ ID NO: 148 - RNA, 10 nt, synthetic construct
  source 1..10: mol_type = other RNA
gctgacaacc

SEQ ID NO: 149 - RNA, 10 nt, synthetic construct
  source 1..10: mol_type = other RNA
agggaggtgg

SEQ ID NO: 150 - RNA, 10 nt, synthetic construct
  source 1..10: mol_type = other RNA
cccagggagg

SEQ ID NO: 151 - RNA, 10 nt, synthetic construct
  source 1..10: mol_type = other RNA
gggcccaggg

SEQ ID NO: 152 - RNA, 10 nt, synthetic construct
  source 1..10: mol_type = other RNA
cctggggccc

SEQ ID NO: 153 - RNA, 10 nt, synthetic construct
  source 1..10: mol_type = other RNA
agtagtcctg

SEQ ID NO: 154 - RNA, 10 nt, synthetic construct
  source 1..10: mol_type = other RNA
tgagtagtcc

SEQ ID NO: 155 - RNA, 20 nt, synthetic construct
  source 1..20: mol_type = other RNA
agtagtcctggggcccaggg

SEQ ID NO: 156 - RNA, 20 nt, synthetic construct
  source 1..20: mol_type = other RNA
agtgagtagtcctggggccc

SEQ ID NO: 157 - RNA, 20 nt, synthetic construct
  source 1..20: mol_type = other RNA
ccgaggagtgagtagtcctg

SEQ ID NO: 158 - RNA, 20 nt, synthetic construct
  source 1..20: mol_type = other RNA
caccgaggagtgagtagtcc

SEQ ID NO: 159 - RNA, 20 nt, synthetic construct
  source 1..20: mol_type = other RNA
tggggtgattatgagcaccg

SEQ ID NO: 160 - RNA, 20 nt, synthetic construct
  source 1..20: mol_type = other RNA
gccgtggtcctggggccgtg

SEQ ID NO: 161 - RNA, 20 nt, synthetic construct
  source 1..20: mol_type = other RNA
aggttggtgccgtggtcctg

SEQ ID NO: 162 - RNA, 10 nt, synthetic construct
  source 1..10: mol_type = other RNA
gggcccaggg

SEQ ID NO: 163 - RNA, 10 nt, synthetic construct
  source 1..10: mol_type = other RNA
cctggggccc

SEQ ID NO: 164 - RNA, 10 nt, synthetic construct
  source 1..10: mol_type = other RNA
agtagtcctg

SEQ ID NO: 165 - RNA, 10 nt, synthetic construct
  source 1..10: mol_type = other RNA
tgagtagtcc

SEQ ID NO: 166 - RNA, 10 nt, synthetic construct
  source 1..10: mol_type = other RNA
atgagcaccg

SEQ ID NO: 167 - RNA, 10 nt, synthetic construct
  source 1..10: mol_type = other RNA
tggggccgtg

SEQ ID NO: 168 - RNA, 10 nt, synthetic construct
  source 1..10: mol_type = other RNA
cgtggtcctg

SEQ ID NO: 169 - RNA, 20 nt, synthetic construct
  source 1..20: mol_type = other RNA
tggggaaacgagggtcagct

SEQ ID NO: 170 - RNA, 20 nt, synthetic construct
  source 1..20: mol_type = other RNA
ggacccctgtggggaaacga

SEQ ID NO: 171 - RNA, 20 nt, synthetic construct
  source 1..20: mol_type = other RNA
ccatagccaggacccctgtg

SEQ ID NO: 172 - RNA, 20 nt, synthetic construct
  source 1..20: mol_type = other RNA
atccatagccaggacccctg

SEQ ID NO: 173 - RNA, 20 nt, synthetic construct
  source 1..20: mol_type = other RNA
agaaatttggatccatagcc

SEQ ID NO: 174 - RNA, 10 nt, synthetic construct
  source 1..10: mol_type = other RNA
agggtcagct

SEQ ID NO: 175 - RNA, 10 nt, synthetic construct
  source 1..10: mol_type = other RNA
ggggaaacga

SEQ ID NO: 176 - RNA, 10 nt, synthetic construct
  source 1..10: mol_type = other RNA
gacccctgtg

SEQ ID NO: 177 - RNA, 10 nt, synthetic construct
  source 1..10: mol_type = other RNA
aggacccctg

SEQ ID NO: 178 - RNA, 10 nt, synthetic construct
  source 1..10: mol_type = other RNA
atccatagcc

SEQ ID NO: 179 - RNA, 20 nt, synthetic construct
  source 1..20: mol_type = other RNA
gttctagagtgccagggatg

SEQ ID NO: 180 - RNA, 20 nt, synthetic construct
  source 1..20: mol_type = other RNA
ggccgggttctagagtgcca

SEQ ID NO: 181 - RNA, 20 nt, synthetic construct
  source 1..20: mol_type = other RNA
tggccgggttctagagtgcc

SEQ ID NO: 182 - RNA, 20 nt, synthetic construct
  source 1..20: mol_type = other RNA
gtcaggtttttggagtggcc

SEQ ID NO: 183 - RNA, 20 nt, synthetic construct
  source 1..20: mol_type = other RNA
agcaggtcaggtttttggag

SEQ ID NO: 184 - RNA, 20 nt, synthetic construct
  source 1..20: mol_type = other RNA
cacagagcaggtcaggtttt

SEQ ID NO: 185 - RNA, 20 nt, synthetic construct
  source 1..20: mol_type = other RNA
cccaggacacagagcaggtc

SEQ ID NO: 186 - RNA, 20 nt, synthetic construct
  source 1..20: mol_type = other RNA
acaggcccaggacacagagc

SEQ ID NO: 187 - RNA, 10 nt, synthetic construct
  source 1..10: mol_type = other RNA
gccagggatg

SEQ ID NO: 188 - RNA, 10 nt, synthetic construct
  source 1..10: mol_type = other RNA
tagagtgcca

SEQ ID NO: 189 - RNA, 10 nt, synthetic construct
  source 1..10: mol_type = other RNA
ctagagtgcc

SEQ ID NO: 190 - RNA, 10 nt, synthetic construct
  source 1..10: mol_type = other RNA
tggagtggcc

SEQ ID NO: 191 - RNA, 10 nt, synthetic construct
  source 1..10: mol_type = other RNA
gtttttggag

SEQ ID NO: 192 - RNA, 10 nt, synthetic construct
  source 1..10: mol_type = other RNA
gtcaggtttt

SEQ ID NO: 193 - RNA, 10 nt, synthetic construct
  source 1..10: mol_type = other RNA
agagcaggtc

SEQ ID NO: 194 - RNA, 10 nt, synthetic construct
  source 1..10: mol_type = other RNA
gacacagagc

SEQ ID NO: 195 - RNA, 20 nt, synthetic construct
  source 1..20: mol_type = other RNA
ctgtctcccctacaccctca

SEQ ID NO: 196 - RNA, 20 nt, synthetic construct
  source 1..20: mol_type = other RNA
cctgtctcccctacaccctc

SEQ ID NO: 197 - RNA, 10 nt, synthetic construct
  source 1..10: mol_type = other RNA
tacaccctca

SEQ ID NO: 198 - RNA, 10 nt, synthetic construct
  source 1..10: mol_type = other RNA
ctacaccctc

SEQ ID NO: 199 - RNA, 20 nt, synthetic construct
  source 1..20: mol_type = other RNA
ttcctacctgagccatctcc

SEQ ID NO: 200 - RNA, 20 nt, synthetic construct
  source 1..20: mol_type = other RNA
agtaacagccccaggcgggg

SEQ ID NO: 201 - RNA, 20 nt, synthetic construct
  source 1..20: mol_type = other RNA
gtcagtaacagccccaggcg

SEQ ID NO: 202 - RNA, 20 nt, synthetic construct
  source 1..20: mol_type = other RNA
atgtcagtaacagccccagg

SEQ ID NO: 203 - RNA, 20 nt, synthetic construct
  source 1..20: mol_type = other RNA
tcaatgtcagtaacagcccc

SEQ ID NO: 204 - RNA, 10 nt, synthetic construct
  source 1..10: mol_type = other RNA
agccatctcc

SEQ ID NO: 205 - RNA, 10 nt, synthetic construct
  source 1..10: mol_type = other RNA
ccaggcgggg

SEQ ID NO: 206 - RNA, 10 nt, synthetic construct
  source 1..10: mol_type = other RNA
gccccaggcg

SEQ ID NO: 207 - RNA, 10 nt, synthetic construct
  source 1..10: mol_type = other RNA
cagccccagg

SEQ ID NO: 208 - RNA, 10 nt, synthetic construct
  source 1..10: mol_type = other RNA
taacagcccc

SEQ ID NO: 209 - RNA, 20 nt, synthetic construct
  source 1..20: mol_type = other RNA
tcaatgtcagtaacagcccc

SEQ ID NO: 210 - RNA, 10 nt, synthetic construct
  source 1..10: mol_type = other RNA
taacagcccc

SEQ ID NO: 211 - RNA, 20 nt, synthetic construct
  source 1..20: mol_type = other RNA
aggtccatcctcttcacctc

SEQ ID NO: 212 - RNA, 20 nt, synthetic construct
  source 1..20: mol_type = other RNA
ctccaggacccttctacacc

SEQ ID NO: 213 - RNA, 20 nt, synthetic construct
  source 1..20: mol_type = other RNA
ccagccctcacagcccctcc

SEQ ID NO: 214 - RNA, 10 nt, synthetic construct
  source 1..10: mol_type = other RNA
tcttcacctc

SEQ ID NO: 215 - RNA, 10 nt, synthetic construct
  source 1..10: mol_type = other RNA
cttctacacc

SEQ ID NO: 216 - RNA, 10 nt, synthetic construct
  source 1..10: mol_type = other RNA
cagcccctcc

SEQ ID NO: 217 - RNA, 20 nt, synthetic construct
  source 1..20: mol_type = other RNA
attcctgcccactgctgtcc

SEQ ID NO: 218 - RNA, 20 nt, synthetic construct
  source 1..20: mol_type = other RNA
ggctgaccctgtggtagggt

SEQ ID NO: 219 - RNA, 20 nt, synthetic construct
  source 1..20: mol_type = other RNA
gggaggctgaccctgtggta

SEQ ID NO: 220 - RNA, 20 nt, synthetic construct
  source 1..20: mol_type = other RNA
caccggggaggctgaccctg

SEQ ID NO: 221 - RNA, 10 nt, synthetic construct
  source 1..10: mol_type = other RNA
actgctgtcc

SEQ ID NO: 222 - RNA, 10 nt, synthetic construct
  source 1..10: mol_type = other RNA
gtggtagggt

SEQ ID NO: 223 - RNA, 10 nt, synthetic construct
  source 1..10: mol_type = other RNA
ccctgtggta

SEQ ID NO: 224 - RNA, 10 nt, synthetic construct
  source 1..10: mol_type = other RNA
gctgaccctg

SEQ ID NO: 225 - RNA, 20 nt, synthetic construct
  source 1..20: mol_type = other RNA
cgttcccgatggtcctcctt

SEQ ID NO: 226 - RNA, 20 nt, synthetic construct
  source 1..20: mol_type = other RNA
ttccggagctgcgttcccga

SEQ ID NO: 227 - RNA, 20 nt, synthetic construct
  source 1..20: mol_type = other RNA
ggcacctctgtcctgcgttc

SEQ ID NO: 228 - RNA, 10 nt, synthetic construct
  source 1..10: mol_type = other RNA
ggtcctcctt

SEQ ID NO: 229 - RNA, 10 nt, synthetic construct
  source 1..10: mol_type = other RNA
gcgttcccga

SEQ ID NO: 230 - RNA, 10 nt, synthetic construct
  source 1..10: mol_type = other RNA
tcctgcgttc

SEQ ID NO: 231 - RNA, 20 nt, synthetic construct
  source 1..20: mol_type = other RNA
atgctcagggaacacgtatc

SEQ ID NO: 232 - RNA, 20 nt, synthetic construct
  source 1..20: mol_type = other RNA
cactacaaaacggatgctca

SEQ ID NO: 233 - RNA, 20 nt, synthetic construct
  source 1..20: mol_type = other RNA
cgagtgtcttcactacaaaa

SEQ ID NO: 234 - RNA, 10 nt, synthetic construct
  source 1..10: mol_type = other RNA
aacacgtatc

SEQ ID NO: 235 - RNA, 10 nt, synthetic construct
  source 1..10: mol_type = other RNA
cggatgctca

SEQ ID NO: 236 - RNA, 10 nt, synthetic construct
  source 1..10: mol_type = other RNA
cactacaaaa

SEQ ID NO: 237 - RNA, 20 nt, synthetic construct
  source 1..20: mol_type = other RNA
accttaccgcttaccgcagc

SEQ ID NO: 238 - RNA, 20 nt, synthetic construct
  source 1..20: mol_type = other RNA
gtgcgggtgccatcggcgtg

SEQ ID NO: 239 - RNA, 10 nt, synthetic construct
  source 1..10: mol_type = other RNA
ttaccgcagc

SEQ ID NO: 240 - RNA, 10 nt, synthetic construct
  source 1..10: mol_type = other RNA
catcggcgtg

SEQ ID NO: 241 - RNA, 20 nt, synthetic construct
  source 1..20: mol_type = other RNA
accttaccgcttaccgcagc

SEQ ID NO: 242 - RNA, 20 nt, synthetic construct
  source 1..20: mol_type = other RNA
gtgcgggtgccatcggcgtg

SEQ ID NO: 243 - RNA, 10 nt, synthetic construct
  source 1..10: mol_type = other RNA
ttaccgcagc

SEQ ID NO: 244 - RNA, 10 nt, synthetic construct
  source 1..10: mol_type = other RNA
catcggcgtg

SEQ ID NO: 245 - RNA, 20 nt, synthetic construct
  source 1..20: mol_type = other RNA
cggggcaggagcgcagcctt

SEQ ID NO: 246 - RNA, 20 nt, synthetic construct
  source 1..20: mol_type = other RNA
cccacatcctggtgcggggc

SEQ ID NO: 247 - RNA, 20 nt, synthetic construct
  source 1..20: mol_type = other RNA
agttcccacatcctggtgcg

SEQ ID NO: 248 - RNA, 20 nt, synthetic construct
  source 1..20: mol_type = other RNA
aaagttcccacatcctggtg

SEQ ID NO: 249 - RNA, 20 nt, synthetic construct
  source 1..20: mol_type = other RNA
ttctcaaagttcccacatcc

SEQ ID NO: 250 - RNA, 10 nt, synthetic construct
  source 1..10: mol_type = other RNA
gcgcagcctt

SEQ ID NO: 251 - RNA, 10 nt, synthetic construct
  source 1..10: mol_type = other RNA
ggtgcggggc

SEQ ID NO: 252 - RNA, 10 nt, synthetic construct
  source 1..10: mol_type = other RNA
tcctggtgcg

SEQ ID NO: 253 - RNA, 10 nt, synthetic construct
  source 1..10: mol_type = other RNA
catcctggtg

SEQ ID NO: 254 - RNA, 10 nt, synthetic construct
  source 1..10: mol_type = other RNA
tcccacatcc

SEQ ID NO: 255 - RNA, 20 nt, synthetic construct
  source 1..20: mol_type = other RNA
gatgctcagggaacacgtat

SEQ ID NO: 256 - RNA, 20 nt, synthetic construct
  source 1..20: mol_type = other RNA
cactacaaaacggatgctca

SEQ ID NO: 257 - RNA, 20 nt, synthetic construct
  source 1..20: mol_type = other RNA
cgagtgtcttcactacaaaa

SEQ ID NO: 258 - RNA, 10 nt, synthetic construct
  source 1..10: mol_type = other RNA
gaacacgtat

SEQ ID NO: 259 - RNA, 10 nt, synthetic construct
  source 1..10: mol_type = other RNA
cggatgctca

SEQ ID NO: 260 - RNA, 10 nt, synthetic construct
  source 1..10: mol_type = other RNA
cactacaaaa

SEQ ID NO: 261 - RNA, 20 nt, synthetic construct
  source 1..20: mol_type = other RNA
cgtttgcaggagacagggca

SEQ ID NO: 262 - RNA, 20 nt, synthetic construct
  source 1..20: mol_type = other RNA
tcgtttgcaggagacagggc

SEQ ID NO: 263 - RNA, 20 nt, synthetic construct
  source 1..20: mol_type = other RNA
tccttcgtttgcaggagaca

SEQ ID NO: 264 - RNA, 20 nt, synthetic construct
  source 1..20: mol_type = other RNA
ttccttcgtttgcaggagac

SEQ ID NO: 265 - RNA, 20 nt, synthetic construct
  source 1..20: mol_type = other RNA
tcttaccttccttcgtttgc

SEQ ID NO: 266 - RNA, 10 nt, synthetic construct
  source 1..10: mol_type = other RNA
agacagggca

SEQ ID NO: 267 - RNA, 10 nt, synthetic construct
  source 1..10: mol_type = other RNA
gagacagggc

SEQ ID NO: 268 - RNA, 10 nt, synthetic construct
  source 1..10: mol_type = other RNA
gcaggagaca

SEQ ID NO: 269 - RNA, 10 nt, synthetic construct
  source 1..10: mol_type = other RNA
tgcaggagac

SEQ ID NO: 270 - RNA, 10 nt, synthetic construct
  source 1..10: mol_type = other RNA
cttcgtttgc

SEQ ID NO: 271 - RNA, 20 nt, synthetic construct
  source 1..20: mol_type = other RNA
atccacgtcatgaatccagc

SEQ ID NO: 272 - RNA, 20 nt, synthetic construct
  source 1..20: mol_type = other RNA
ggacgtccgcgggggccccc

SEQ ID NO: 273 - RNA, 10 nt, synthetic construct
  source 1..10: mol_type = other RNA
tgaatccagc

SEQ ID NO: 274 - RNA, 10 nt, synthetic construct
  source 1..10: mol_type = other RNA
gggggccccc

SEQ ID NO: 275 - RNA, 20 nt, synthetic construct
  source 1..20: mol_type = other RNA
tacccccaccgctccccagg

SEQ ID NO: 276 - RNA, 20 nt, synthetic construct
  source 1..20: mol_type = other RNA
gtctacccccaccgctcccc

SEQ ID NO: 277 - RNA, 10 nt, synthetic construct
  source 1..10: mol_type = other RNA
gctccccagg

SEQ ID NO: 278 - RNA, 10 nt, synthetic construct
  source 1..10: mol_type = other RNA
accgctcccc

SEQ ID NO: 279 - RNA, 20 nt, synthetic construct
  source 1..20: mol_type = other RNA
gagcctttgctttcatcctt

SEQ ID NO: 280 - RNA, 20 nt, synthetic construct
  source 1..20: mol_type = other RNA
cacatttctgttaaggtccc

SEQ ID NO: 281 - RNA, 20 nt, synthetic construct
  source 1..20: mol_type = other RNA
tatcggtcacatttctgtta

SEQ ID NO: 282 - RNA, 10 nt, synthetic construct
  source 1..10: mol_type = other RNA
tttcatcctt

SEQ ID NO: 283 - RNA, 10 nt, synthetic construct
  source 1..10: mol_type = other RNA
ttaaggtccc

SEQ ID NO: 284 - RNA, 10 nt, synthetic construct
  source 1..10: mol_type = other RNA
atttctgtta

SEQ ID NO: 285 - RNA, 20 nt, synthetic construct
  source 1..20: mol_type = other RNA
ctttgcaatgtcaagtacag

SEQ ID NO: 286 - RNA, 10 nt, synthetic construct
  source 1..10: mol_type = other RNA
tcaagtacag

SEQ ID NO: 287 - RNA, 20 nt, synthetic construct
  source 1..20: mol_type = other RNA
gatcagcagcgacgtccgcc

SEQ ID NO: 288 - RNA, 20 nt, synthetic construct
  source 1..20: mol_type = other RNA
gatcacgaagacacagacca

SEQ ID NO: 289 - RNA, 10 nt, synthetic construct
  source 1..10: mol_type = other RNA
gacgtccgcc

SEQ ID NO: 290 - RNA, 10 nt, synthetic construct
  source 1..10: mol_type = other RNA
acacagacca

SEQ ID NO: 291 - RNA, 20 nt, synthetic construct
  source 1..20: mol_type = other RNA
cgatggggtctttcatgtga

SEQ ID NO: 292 - RNA, 20 nt, synthetic construct
  source 1..20: mol_type = other RNA
ccgatggggtctttcatgtg

SEQ ID NO: 293 - RNA, 20 nt, synthetic construct
  source 1..20: mol_type = other RNA
tttggaagctgtcaccgatg

SEQ ID NO: 294 - RNA, 20 nt, synthetic construct
  source 1..20: mol_type = other RNA
gttttggaagctgtcaccga

SEQ ID NO: 295 - RNA, 20 nt, synthetic construct
  source 1..20: mol_type = other RNA
aacataccagcttgtcgttt

SEQ ID NO: 296 - RNA, 10 nt, synthetic construct
  source 1..10: mol_type = other RNA
tttcatgtga

SEQ ID NO: 297 - RNA, 10 nt, synthetic construct
  source 1..10: mol_type = other RNA
ctttcatgtg

SEQ ID NO: 298 - RNA, 10 nt, synthetic construct
  source 1..10: mol_type = other RNA
gtcaccgatg

SEQ ID NO: 299 - RNA, 10 nt, synthetic construct
  source 1..10: mol_type = other RNA
ctgtcaccga

SEQ ID NO: 300 - RNA, 10 nt, synthetic construct
  source 1..10: mol_type = other RNA
cttgtcgttt

SEQ ID NO: 301 - RNA, 20 nt, synthetic construct
  source 1..20: mol_type = other RNA
acaggagtatagcaaaaatt

SEQ ID NO: 302 - RNA, 20 nt, synthetic construct
  source 1..20: mol_type = other RNA
aacaggagtatagcaaaaat

SEQ ID NO: 303 - RNA, 20 nt, synthetic construct
  source 1..20: mol_type = other RNA
taccaaactgtccccagaac

SEQ ID NO: 304 - RNA, 10 nt, synthetic construct
  source 1..10: mol_type = other RNA
agcaaaaatt

SEQ ID NO: 305 - RNA, 10 nt, synthetic construct
  source 1..10: mol_type = other RNA
tagcaaaaat

SEQ ID NO: 306 - RNA, 10 nt, synthetic construct
  source 1..10: mol_type = other RNA
tccccagaac

SEQ ID NO: 307 - RNA, 20 nt, synthetic construct
  source 1..20: mol_type = other RNA
tacctcctgcgttcctgcct

SEQ ID NO: 308 - RNA, 20 nt, synthetic construct
  source 1..20: mol_type = other RNA
taatgcagccctcctcacct

SEQ ID NO: 309 - RNA, 10 nt, synthetic construct
  source 1..10: mol_type = other RNA
gttcctgcct

SEQ ID NO: 310 - RNA, 10 nt, synthetic construct
  source 1..10: mol_type = other RNA
ctcctcacct

SEQ ID NO: 311 - RNA, 20 nt, synthetic construct
  source 1..20: mol_type = other RNA
aaccaatatggcaatgacga

SEQ ID NO: 312 - RNA, 20 nt, synthetic construct
  source 1..20: mol_type = other RNA
tatcacctgaataaccaata

SEQ ID NO: 313 - RNA, 20 nt, synthetic construct
  source 1..20: mol_type = other RNA
tccaaccacagcgaggatat

SEQ ID NO: 314 - RNA, 20 nt, synthetic construct
  source 1..20: mol_type = other RNA
gactcagtccaaccacagcg

SEQ ID NO: 315 - RNA, 10 nt, synthetic construct
  source 1..10: mol_type = other RNA
gcaatgacga

SEQ ID NO: 316 - RNA, 10 nt, synthetic construct
  source 1..10: mol_type = other RNA
ataaccaata

SEQ ID NO: 317 - RNA, 10 nt, synthetic construct
  source 1..10: mol_type = other RNA
gcgaggatat

SEQ ID NO: 318 - RNA, 10 nt, synthetic construct
  source 1..10: mol_type = other RNA
aaccacagcg

SEQ ID NO: 319 - RNA, 20 nt, synthetic construct
  source 1..20: mol_type = other RNA
cttattcttttaacaggtcc

SEQ ID NO: 320 - RNA, 20 nt, synthetic construct
  source 1..20: mol_type = other RNA
aacattcttattcttttaac

SEQ ID NO: 321 - RNA, 10 nt, synthetic construct
  source 1..10: mol_type = other RNA
taacaggtcc

SEQ ID NO: 322 - RNA, 10 nt, synthetic construct
  source 1..10: mol_type = other RNA
ttcttttaac

SEQ ID NO: 323 - RNA, 20 nt, synthetic construct
  source 1..20: mol_type = other RNA
tgatctcactttcctacctt

SEQ ID NO: 324 - RNA, 10 nt, synthetic construct
  source 1..10: mol_type = other RNA
ttcctacctt

SEQ ID NO: 325 - RNA, 20 nt, synthetic construct
  source 1..20: mol_type = other RNA
ttttgtctaaaaagagctac

SEQ ID NO: 326 - RNA, 10 nt, synthetic construct
  source 1..10: mol_type = other RNA
aaagagctac

SEQ ID NO: 327 - RNA, 20 nt, synthetic construct
  source 1..20: mol_type = other RNA
atcctttgcatatttcaaat

SEQ ID NO: 328 - RNA, 10 nt, synthetic construct
  source 1..10: mol_type = other RNA
tatttcaaat

SEQ ID NO: 329 - RNA, 20 nt, synthetic construct
  source 1..20: mol_type = other RNA
gcagcttcctgcagaaccca

SEQ ID NO: 330 - RNA, 20 nt, synthetic construct
  source 1..20: mol_type = other RNA
ccaatatacaaactggagtg

SEQ ID NO: 331 - RNA, 10 nt, synthetic construct
  source 1..10: mol_type = other RNA
gcagaaccca

SEQ ID NO: 332 - RNA, 10 nt, synthetic construct
  source 1..10: mol_type = other RNA
aactggagtg

SEQ ID NO: 333 - RNA, 20 nt, synthetic construct
  source 1..20: mol_type = other RNA
tgatttctggaggatgattt

SEQ ID NO: 334 - RNA, 20 nt, synthetic construct
  source 1..20: mol_type = other RNA
aaactgttgatgatttctgg

SEQ ID NO: 335 - RNA, 20 nt, synthetic construct
  source 1..20: mol_type = other RNA
tgtttacctcctaggtccca

SEQ ID NO: 336 - RNA, 10 nt, synthetic construct
  source 1..10: mol_type = other RNA
aggatgattt

SEQ ID NO: 337 - RNA, 10 nt, synthetic construct
  source 1..10: mol_type = other RNA
tgatttctgg

SEQ ID NO: 338 - RNA, 10 nt, synthetic construct
  source 1..10: mol_type = other RNA
ctaggtccca

SEQ ID NO: 339 - RNA, 20 nt, synthetic construct
  source 1..20: mol_type = other RNA
cctggaagccgagaacaaag

SEQ ID NO: 340 - RNA, 10 nt, synthetic construct
  source 1..10: mol_type = other RNA
gagaacaaag

SEQ ID NO: 341 - RNA, 20 nt, synthetic construct
  source 1..20: mol_type = other RNA
gggccattacggtccctggg

SEQ ID NO: 342 - RNA, 10 nt, synthetic construct
  source 1..10: mol_type = other RNA
ggtccctggg

SEQ ID NO: 343 - RNA, 20 nt, synthetic construct
  source 1..20: mol_type = other RNA
aatcatagagattaattcct

SEQ ID NO: 344 - RNA, 10 nt, synthetic construct
  source 1..10: mol_type = other RNA
attaattcct

SEQ ID NO: 345 - RNA, 20 nt, synthetic construct
  source 1..20: mol_type = other RNA
gtcacttgaaagtggaacac

SEQ ID NO: 346 - RNA, 20 nt, synthetic construct
  source 1..20: mol_type = other RNA
ggtaaggggtcacttgaaag

SEQ ID NO: 347 - RNA, 20 nt, synthetic construct
  source 1..20: mol_type = other RNA
gcagtggtgtgagtaggtaa

SEQ ID NO: 348 - RNA, 20 nt, synthetic construct
  source 1..20: mol_type = other RNA
gagaatgcagtggtgtgagt

SEQ ID NO: 349 - RNA, 20 nt, synthetic construct
  source 1..20: mol_type = other RNA
gcttgcgggtgagaatgcag

SEQ ID NO: 350 - RNA, 10 nt, synthetic construct
  source 1..10: mol_type = other RNA
agtggaacac

SEQ ID NO: 351 - RNA, 10 nt, synthetic construct
  source 1..10: mol_type = other RNA
cacttgaaag

SEQ ID NO: 352 - RNA, 10 nt, synthetic construct
  source 1..10: mol_type = other RNA
gagtaggtaa

SEQ ID NO: 353 - RNA, 10 nt, synthetic construct
  source 1..10: mol_type = other RNA
tggtgtgagt

SEQ ID NO: 354 - RNA, 10 nt, synthetic construct
  source 1..10: mol_type = other RNA
gagaatgcag

SEQ ID NO: 355 - RNA, 20 nt, synthetic construct
  source 1..20: mol_type = other RNA
tgggtctgtaggaaaggtgc

SEQ ID NO: 356 - RNA, 20 nt, synthetic construct
  source 1..20: mol_type = other RNA
ggaaactgggtctgtaggaa

SEQ ID NO: 357 - RNA, 20 nt, synthetic construct
  source 1..20: mol_type = other RNA
aatggggaaactgggtctgt

SEQ ID NO: 358 - RNA, 20 nt, synthetic construct
  source 1..20: mol_type = other RNA
tggttgtcaatggggaaact

SEQ ID NO: 359 - RNA, 20 nt, synthetic construct
  source 1..20: mol_type = other RNA
gctgagggtggttgtcaatg

SEQ ID NO: 360 - RNA, 20 nt, synthetic construct
  source 1..20: mol_type = other RNA
aggctgagggtggttgtcaa

SEQ ID NO: 361 - RNA, 20 nt, synthetic construct
  source 1..20: mol_type = other RNA
gtggtgtgcaaggctgaggg

SEQ ID NO: 362 - RNA, 20 nt, synthetic construct
  source 1..20: mol_type = other RNA
agctgtggtgtgcaaggctg

SEQ ID NO: 363 - RNA, 20 nt, synthetic construct
  source 1..20: mol_type = other RNA
cagcagagctgtggtgtgca

SEQ ID NO: 364 - RNA, 10 nt, synthetic construct
  source 1..10: mol_type = other RNA
ggaaaggtgc

SEQ ID NO: 365 - RNA, 10 nt, synthetic construct
  source 1..10: mol_type = other RNA
tctgtaggaa

SEQ ID NO: 366 - RNA, 10 nt, synthetic construct
  source 1..10: mol_type = other RNA
ctgggtctgt

SEQ ID NO: 367 - RNA, 10 nt, synthetic construct
  source 1..10: mol_type = other RNA
tggggaaact

SEQ ID NO: 368 - RNA, 10 nt, synthetic construct
  source 1..10: mol_type = other RNA
gttgtcaatg

SEQ ID NO: 369 - RNA, 10 nt, synthetic construct
  source 1..10: mol_type = other RNA
tggttgtcaa

SEQ ID NO: 370 - RNA, 10 nt, synthetic construct
  source 1..10: mol_type = other RNA
aggctgaggg

SEQ ID NO: 371 - RNA, 10 nt, synthetic construct
  source 1..10: mol_type = other RNA
tgcaaggctg

SEQ ID NO: 372 - RNA, 10 nt, synthetic construct
  source 1..10: mol_type = other RNA
gtggtgtgca

SEQ ID NO: 373 - RNA, 20 nt, synthetic construct
  source 1..20: mol_type = other RNA
ccaggcacatggggatggtg

SEQ ID NO: 374 - RNA, 20 nt, synthetic construct
  source 1..20: mol_type = other RNA
catcaccaggcacatgggga

SEQ ID NO: 375 - RNA, 20 nt, synthetic construct
  source 1..20: mol_type = other RNA
gagcacatcaccaggcacat

SEQ ID NO: 376 - RNA, 20 nt, synthetic construct
  source 1..20: mol_type = other RNA
ccttgtgagagcacatcacc

SEQ ID NO: 377 - RNA, 20 nt, synthetic construct
  source 1..20: mol_type = other RNA
cccgaggtagagtgggtgga

SEQ ID NO: 378 - RNA, 10 nt, synthetic construct
  source 1..10: mol_type = other RNA
ggggatggtg

SEQ ID NO: 379 - RNA, 10 nt, synthetic construct
  source 1..10: mol_type = other RNA
cacatgggga

SEQ ID NO: 380 - RNA, 10 nt, synthetic construct
  source 1..10: mol_type = other RNA
ccaggcacat

SEQ ID NO: 381 - RNA, 10 nt, synthetic construct
  source 1..10: mol_type = other RNA
gcacatcacc

SEQ ID NO: 382 - RNA, 10 nt, synthetic construct
  source 1..10: mol_type = other RNA
agtgggtgga

SEQ ID NO: 383 - RNA, 20 nt, synthetic construct
  source 1..20: mol_type = other RNA
taaagtgctattccaaagtg

SEQ ID NO: 384 - RNA, 10 nt, synthetic construct
  source 1..10: mol_type = other RNA
ttccaaagtg

SEQ ID NO: 385 - RNA, 20 nt, synthetic construct
  source 1..20: mol_type = other RNA
aaggagatggatttggcata

SEQ ID NO: 386 - RNA, 20 nt, synthetic construct
  source 1..20: mol_type = other RNA
cttgtccaaggagatggatt

SEQ ID NO: 387 - RNA, 20 nt, synthetic construct
  source 1..20: mol_type = other RNA
gtaagccttgtccaaggaga

SEQ ID NO: 388 - RNA, 20 nt, synthetic construct
  source 1..20: mol_type = other RNA
acaaatgtaagccttgtcca

SEQ ID NO: 389 - RNA, 10 nt, synthetic construct
  source 1..10: mol_type = other RNA
atttggcata

SEQ ID NO: 390 - RNA, 10 nt, synthetic construct
  source 1..10: mol_type = other RNA
gagatggatt

SEQ ID NO: 391 - RNA, 10 nt, synthetic construct
  source 1..10: mol_type = other RNA
tccaaggaga

SEQ ID NO: 392 - RNA, 10 nt, synthetic construct
  source 1..10: mol_type = other RNA
gccttgtcca

SEQ ID NO: 393 - RNA, 20 nt, synthetic construct
  source 1..20: mol_type = other RNA
atgtttggacatcatcactt

SEQ ID NO: 394 - RNA, 20 nt, synthetic construct
  source 1..20: mol_type = other RNA
ttttactctcctgccatgtt

SEQ ID NO: 395 - RNA, 20 nt, synthetic construct
  source 1..20: mol_type = other RNA
gctggcattctgagcagcac

SEQ ID NO: 396 - RNA, 10 nt, synthetic construct
  source 1..10: mol_type = other RNA
atcatcactt

SEQ ID NO: 397 - RNA, 10 nt, synthetic construct
  source 1..10: mol_type = other RNA
ctgccatgtt

SEQ ID NO: 398 - RNA, 10 nt, synthetic construct
  source 1..10: mol_type = other RNA
tgagcagcac

SEQ ID NO: 399 - RNA, 20 nt, synthetic construct
  source 1..20: mol_type = other RNA
atgtttggacatcatcactt

SEQ ID NO: 400 - RNA, 20 nt, synthetic construct
  source 1..20: mol_type = other RNA
ttttactctcctgccatgtt

SEQ ID NO: 401 - RNA, 20 nt, synthetic construct
  source 1..20: mol_type = other RNA
gctggcattctgagcagcac

SEQ ID NO: 402 - RNA, 10 nt, synthetic construct
  source 1..10: mol_type = other RNA
atcatcactt

SEQ ID NO: 403 - RNA, 10 nt, synthetic construct
  source 1..10: mol_type = other RNA
ctgccatgtt

SEQ ID NO: 404 - RNA, 10 nt, synthetic construct
  source 1..10: mol_type = other RNA
tgagcagcac

(19) United States
(12) Patent Application Publication
(10) Pub. No.: US 2025/0019724 A1
(43) Pub. Date: Jan. 16, 2025

(54) REGENERATION OF SURFACE ANTIGEN-NEGATIVE CELLS

(71) Applicant: CORRECTSEQUENCE THERAPEUTICS, Pudong District, Shanghai (CN)

(72) Inventors: Lijie WANG, Shanghai (CN); Yichuan WANG, Shanghai (CN); Suling LI, Shanghai (CN); Jia CHEN, Shanghai (CN); Li YANG, Shanghai (CN)

(21) Appl. No.: 18/711,858

(22) PCT Filed: Nov. 18, 2022
(86) PCT No.: PCT/CN2022/132953

§ 371 Date: May 20, 2024

(30) Foreign Application Priority Data
Nov. 18, 2021 (WO) PCT/CN2021/131565

(51) Int. Cl. C12N 15/90; C12N 9/22; C12N 9/78; C12N 15/11

(52) U.S. Cl. CPC C12N 15/907; C12N 9/22; C12N 9/78; C12N 15/11; C12Y 305/04005; C07K 2319/50; C12N 2310/20

(57) ABSTRACT

Provided are gene editing technologies, including specifically designed and tested guide RNA sequences for improved base editors, useful for disrupting the expression of genes, such as CD33, CD123, CD47, CD45 and CLL1, in a cell. Such methods and edited cells are useful in reducing the toxicity associated with therapies targeting such cell surface antigens, such as those for treating acute myeloid leukemia.

### BACKGROUND

Acute myeloid leukemia is a common acute leukemia in adults and children. Targeted therapy gradually has been one of the major ways of AML treatments. AML cell surface antigens are shared with normal myeloid progenitors, in other words, some surface markers that found on AML cells can also be found on normal cells. Therefore, targeting AML tumor cells based on surface marker also result in toxicity to myeloid system, limiting the use in clinical trials. The new approaches of targeted therapy are required to target AML tumor cells while retaining normal hematopoietic system unaffected.

Currently, there is a new paradigm for antigen-specific targeted therapeutics: regenerating a surface antigen-negative myeloid system that is resistant to targeted therapy by using genome-edited hematopoietic stem and progenitor cells (HSPCs).

CD33 (Siglec-3) is a member of the sialic acid-binding immunoglobulin-like lectin family. CD123 (IL-3Ra) is a receptor for interleukin-3. Both CD33 and CD123 are major AML cell-specific antigens and therapeutic target for AML, but their expression on normal myeloid cells limits the therapy window. Besides CD33 and CD123, many cell surface antigens that are expressed on both normal stem cells and leukemic stem cells, including CD47/IAP (integrin associated protein), CD45 (common leukocyte antigen) and CLL-1 (C-type lectin protein-1). Therefore, targeting AML based on these surface markers often comes at a risk of myelosuppression. It's a limitation of clinical applications owing to adverse life-threatening reactions. Cas9 nucleases have been applied to disrupt CD33 gene in the primary cells. CD33-null human HSPCs remain functional and proliferating while being resistant to CD33-targeted AML therapy, e.g., antibody drug conjugate (ADC) therapeutics. Gene editing tools are likely to make the surface antigens promising AML therapy targets.

The combination of CRISPR-Cas9 and cytidine deaminases leads to cytosine base editors (CBEs) for programmable cytosine to thymine (C-T) substitution, which has been applied to achieve efficient editing in various species successfully and holds great potentials in clinical applications. As base editor avoids inducing DNA double strand break (DSB), unwanted nucleotide insertions/deletions (indels) or DNA damage responses (DDRs) can be largely avoided.

The safety and efficiency of gene editing tools are of great importance in clinical applications. Previous studies have reported that the DSBs induced by Cas9 nuclease can activate a p53-mediated DDR pathway and then lead to cell death. Moreover, APOBEC/AID family members can trigger C-to-T base substitutions in single-stranded DNA (ssDNA) regions, which are formed randomly during various cellular processes including DNA replication, repair and transcription. Thus, the specificity of previous base editing systems is compromised, limiting the applications of BEs for therapeutic purposes.

### SUMMARY

The instant disclosure, in some embodiments, describes gene editing technologies, including specifically designed and tested guide RNA sequences for improved base editors, useful for disrupting the expression of genes, such as CD33, CD123, CD47, CD45 and CLL1, in a cell. Such methods and edited cells are useful in reducing the toxicity associated with therapies targeting such cell surface antigens, such as those for treating acute myeloid leukemia.

One embodiment of the present disclosure, accordingly, provides a method for reducing the biological activity of a gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides.

The gene, in some embodiments, is a surface antigen expressed on a cancer cell but is also expressed in a non-cancerous cell, such as CD33, CD123, CD47, CD45, and CLL1. Example sgRNA and the hsgRNA are provided in Tables 1A-1M. In some embodiments, the hsgRNA comprises a corresponding 10-nt sequence listed therein. In some embodiments, the hsgRNA comprises a corresponding 20-nt sequence listed therein.

In some embodiments, the nucleobase deaminase is a cytidine deaminase, such as APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4) and AICDA (AID).

In some embodiments, the method further comprises introducing into the cell a nucleobase deaminase inhibitor, fused to the nucleobase deaminase, via a protease cleavage site. In some embodiments, the nucleobase deaminase inhibitor is an inhibitory domain of a nucleobase deaminase. In some embodiments, the nucleobase deaminase inhibitor is an inhibitory domain of a cytidine deaminase.

In some embodiments, the method further comprises introducing into the cell a protease that is capable of cleaving at the protease cleavage site. In some embodiments, the protease is selected from the group consisting of TuMV protease, PPV protease, PVY protease, ZIKV protease and WNV protease.

Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b. In some embodiments, the Cas protein is catalytically impaired, such as nCas9 or dCpf1.

The cell being targeted here, in some embodiments, is a blood cell, such as a myeloid cell, in particular non-cancerous blood cells. In some embodiments, the cell is in vitro, ex vivo, or in vivo in a human patient. In some embodiments, the patient suffers from a cancer.

Also provided, in some embodiments, is one or more polynucleotides encoding a CRISPR-associated (Cas) protein, a nucleobase deaminase, a single-guide RNA (sgRNA), and a helper single-guide RNA (hsgRNA), wherein the sgRNA and the hsgRNA are selected from the sequences from Table 1.

Also provided, in some embodiments, is a cell prepared by the method of the present disclosure, and methods of using the cell. One embodiment provides a method of reducing toxicity in a patient undergoing a therapy targeting a cell surface antigen on a cancer cell, comprising administering to the patient the cell. Another embodiment provides a method of reducing toxicity in a patient undergoing a therapy targeting a cell surface antigen on a cancer cell, comprising administering to the patient the polynucleotides.

Also provided are genomic sequences, mRNA sequences and protein sequences that can be prepared by the disclosed base editing technologies and guide RNA sequences.

### BRIEF DESCRIPTION OF THE DRAWINGS

FIG. 1: Editing efficiencies induced by tBE with the pairs of sgRNA-CD33-CAA-1 and its hsgRNAs targeting human CD33 gene. A: Schematic diagram illustrating the co-transfection of sgRNA-CD33-CAA-1 and its different hsgRNA-CD33-CAA-1s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 2: Editing efficiencies induced by tBE with the pairs of sgRNA-CD33-CAG-2 and its hsgRNAs targeting human CD33 gene. A: Schematic diagram illustrating the co-transfection of sgRNA-CD33-CAG-2 and its different hsgRNA-CD33-CAG-2s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 3: Editing efficiencies induced by tBE with the pairs of sgRNA-CD33-CAG-3 and its hsgRNAs targeting human CD33 gene. A: Schematic diagram illustrating the co-transfection of sgRNA-CD33-CAG-3 and its different hsgRNA-CD33-CAG-3s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 4: Editing efficiencies induced by tBE with the pairs of sgRNA-CD33-CAG-4 and its hsgRNAs targeting human CD33 gene. A: Schematic diagram illustrating the co-transfection of sgRNA-CD33-CAG-4 and its different hsgRNA-CD33-CAG-4s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 5: Editing efficiencies induced by tBE with the pairs of sgRNA-CD33-CAG-5 and its hsgRNAs targeting human CD33 gene. A: Schematic diagram illustrating the co-transfection of sgRNA-CD33-CAG-5 and its different hsgRNA-CD33-CAG-5s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 6: Editing efficiencies induced by tBE with the pairs of sgRNA-CD33-CAG-6 and its hsgRNAs targeting human CD33 gene. A: Schematic diagram illustrating the co-transfection of sgRNA-CD33-CAG-6 and its different hsgRNA-CD33-CAG-6s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 7: Editing efficiencies induced by tBE with the pairs of sgRNA-CD33-TGG-7 and its hsgRNAs targeting human CD33 gene. A: Schematic diagram illustrating the co-transfection of sgRNA-CD33-TGG-7 and its different hsgRNA-CD33-TGG-7s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 8: Editing efficiencies induced by tBE with the pairs of sgRNA-CD33-TGG-8 and its hsgRNAs targeting human CD33 gene. A: Schematic diagram illustrating the co-transfection of sgRNA-CD33-TGG-8 and its different hsgRNA-CD33-TGG-8s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 9: Editing efficiencies induced by tBE with the pairs of sgRNA-CD33-TGG-9 and its hsgRNAs targeting human CD33 gene. A: Schematic diagram illustrating the co-transfection of sgRNA-CD33-TGG-9 and its different hsgRNA-CD33-TGG-9s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 10: Editing efficiencies induced by tBE with the pairs of sgRNA-CD33-TGG-10 and its hsgRNAs targeting human CD33 gene. A: Schematic diagram illustrating the co-transfection of sgRNA-CD33-TGG-10 and its different hsgRNA-CD33-TGG-10s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 11: Editing efficiencies induced by tBE with the pairs of sgRNA-CD33-TGG-11 and its hsgRNAs targeting human CD33 gene. A: Schematic diagram illustrating the co-transfection of sgRNA-CD33-TGG-11 and its different hsgRNA-CD33-TGG-1 Is with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 12: Editing efficiencies induced by tBE with the pairs of sgRNA-CD33-GU-12 and its hsgRNAs targeting human CD33 gene. A: Schematic diagram illustrating the co-transfection of sgRNA-CD33-GU-12 and its different hsgRNA-CD33-GU-12s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 13: Editing efficiencies induced by tBE with the pairs of sgRNA-CD33-AG-13 and its hsgRNAs targeting human CD33 gene. A: Schematic diagram illustrating the co-transfection of sgRNA-CD33-AG-13 and its different hsgRNA-CD33-AG-13s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 14: Editing efficiencies induced by tBE with the pairs of sgRNA-CD33-GU-14 and its hsgRNAs targeting human CD33 gene. A: Schematic diagram illustrating the co-transfection of sgRNA-CD33-GU-14 and its different hsgRNA-CD33-GU-14s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 15: Editing efficiencies induced by tBE with the pair of sgRNA-CD33-AG-15 and its hsgRNA targeting human CD33 gene. A: Schematic diagram illustrating the co-transfection of sgRNA-CD33-AG-15 and its hsgRNA-CD33-AG-15 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 16: Editing efficiencies induced by tBE with the pair of sgRNA-CD33-GU-16 and its hsgRNA targeting human CD33 gene. A: Schematic diagram illustrating the co-transfection of sgRNA-CD33-GU-16 and its hsgRNA-CD33-GU-16 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 17: Editing efficiencies induced by tBE with the pair of sgRNA-CD33-GU-17 and its hsgRNA targeting human CD33 gene. A: Schematic diagram illustrating the co-transfection of sgRNA-CD33-GU-17 and its hsgRNA-CD33-GU-17 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 18: Editing efficiencies induced by tBE with the pair of sgRNA-CD33-AG-18 and its hsgRNA targeting human CD33 gene. A: Schematic diagram illustrating the co-transfection of sgRNA-CD33-AG-18 and its hsgRNA-CD33-AG-18 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 19: Editing efficiencies induced by tBE with the pairs of sgRNA-CD123-CAA-1 and its hsgRNAs targeting human CD123 gene. A: Schematic diagram illustrating the co-transfection of sgRNA-CD123-CAA-1 and its different hsgRNA-CD123-CAA-1s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 20: Editing efficiencies induced by tBE with the pair of sgRNA-CD123-CAA-2 and its hsgRNA targeting human CD123 gene. A: Schematic diagram illustrating the co-transfection of sgRNA-CD123-CAA-2 and its hsgRNA-CD123-CAA-2 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 21: Editing efficiencies induced by tBE with the pair of sgRNA-CD123-CAG-3 and its hsgRNA targeting human CD123 gene. A: Schematic diagram illustrating the co-transfection of sgRNA-CD123-CAG-3 and its hsgRNA-CD123-CAG-3 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 22: Editing efficiencies induced by tBE with the pair of sgRNA-CD123-CAG-4 and its hsgRNA targeting human CD123 gene. A: Schematic diagram illustrating the co-transfection of sgRNA-CD123-CAG-4 and its hsgRNA-CD123-CAG-4 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 23: Editing efficiencies induced by tBE with the pair of sgRNA-CD123-CAG-5 and its hsgRNA targeting human CD123 gene. A: Schematic diagram illustrating the co-transfection of sgRNA-CD123-CAG-5 and its hsgRNA-CD123-CAG-5 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 24: Editing efficiencies induced by tBE with the pairs of sgRNA-CD123-CGA-6 and its hsgRNAs targeting human CD123 gene. A: Schematic diagram illustrating the co-transfection of sgRNA-CD123-CGA-6 and its different hsgRNA-CD123-CGA-6s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 25: Editing efficiencies induced by tBE with the pairs of sgRNA-CD123-TGG-7 and its hsgRNAs targeting human CD123 gene. A: Schematic diagram illustrating the co-transfection of sgRNA-CD123-TGG-7 and its different hsgRNA-CD123-TGG-7s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 26: Editing efficiencies induced by tBE with the pair of sgRNA-CD123-TGG-8 and its hsgRNA targeting human CD123 gene. A: Schematic diagram illustrating the co-transfection of sgRNA-CD123-TGG-8 and its hsgRNA-CD123-TGG-8 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 27: Editing efficiencies induced by tBE with the pairs of sgRNA-CD123-GU-9 and its hsgRNAs targeting human CD123 gene. A: Schematic diagram illustrating the co-transfection of sgRNA-CD123-GU-9 and its different hsgRNA-CD123-GU-9s with tBE-V5-mA3 and nCas9. B: Editing efficiency induced by tBE-V5-mA3 with indicated pairs of sgRNA/hsgRNA and BE4max-YE1 with indicated sgRNA at indicated sites.

FIG. 28: Editing efficiencies induced by tBE with the pair of sgRNA-CD123-AG-10 and its hsgRNA targeting human CD123 gene. A: Schematic diagram illustrating the co-transfection of sgRNA-CD123-AG-10 and its hsgRNA-CD123-AG-10 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 29: Editing efficiencies induced by tBE with the pair of sgRNA-CD123-GU-11 and its hsgRNA targeting human CD123 gene. A: Schematic diagram illustrating the co-transfection of sgRNA-CD123-GU-11 and its hsgRNA-CD123-GU-11 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 30: Editing efficiencies induced by tBE with the pair of sgRNA-CD123-AG-12 and its hsgRNA targeting human CD123 gene. A: Schematic diagram illustrating the co-transfection of sgRNA-CD123-AG-12 and its hsgRNA-CD123-AG-12 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 31: Editing efficiencies induced by tBE with the pair of sgRNA-CD123-AG-13 and its hsgRNA targeting human CD123 gene. A: Schematic diagram illustrating the co-transfection of sgRNA-CD123-AG-13 and its hsgRNA-CD123-AG-13 with tBE-V5-mA3 and nCas9. B: Editing efficiency induced by tBE-V5-mA3 with indicated pair of sgRNA/hsgRNA and BE4max-YE1 with indicated sgRNA at indicated sites.

FIG. 32: Editing efficiencies induced by tBE with the pairs of sgRNA-CD47-TGG-1 and its hsgRNAs targeting human CD47 gene. A: Schematic diagram illustrating the co-transfection of sgRNA-CD47-TGG-1 and its different hsgRNA-CD47-TGG-1s with tBE-V5-mA3 and nCas9. B: Editing efficiency induced by tBE-V5-mA3 with indicated pairs of sgRNA/hsgRNA at indicated sites.

FIG. 33: Editing efficiencies induced by tBE with the pairs of sgRNA-CD47-GU-2 and its hsgRNAs targeting human CD47 gene. A: Schematic diagram illustrating the co-transfection of sgRNA-CD47-GU-2 and its different hsgRNA-CD47-GU-2s with tBE-V5-mA3 and nCas9. B: Editing efficiency induced by tBE-V5-mA3 with indicated pair of sgRNA/hsgRNA at indicated sites.

FIG. 34: Editing efficiencies induced by tBE with the pairs of sgRNA-CD47-AG-3 and its different hsgRNAs targeting human CD47 gene. A: Schematic diagram illustrating the co-transfection of sgRNA-CD47-AG-3 and its different hsgRNA-CD47-AG-3s with tBE-V5-mA3 and nCas9. B: Editing efficiency induced by tBE-V5-mA3 with indicated pairs of sgRNA/hsgRNA at indicated sites.

FIG. 35: Editing efficiencies induced by tBE with the pair of sgRNA-CD45-CAA-1 and its hsgRNA targeting human CD45 gene. A: Schematic diagram illustrating the co-transfection of sgRNA-CD45-CAA-1 and its hsgRNA-CD45-CAA-1 with tBE-V5-mA3 and nCas9. B: Editing efficiency induced by tBE-V5-mA3 with indicated pair of sgRNA/hsgRNA at indicated sites.

FIG. 36: Editing efficiencies induced by tBE with the pair of sgRNA-CD45-CAG-2 and its hsgRNA targeting human CD45 gene. A: Schematic diagram illustrating the co-transfection of sgRNA-CD45-CAG-2 and its hsgRNA-CD45-CAG-2 with tBE-V5-mA3 and nCas9. B: Editing efficiency induced by tBE-V5-mA3 with indicated pair of sgRNA/hsgRNA at indicated sites.

FIG. 37: Editing efficiencies induced by tBE with the pair of sgRNA-CD45-CAG-3 and its hsgRNA targeting human CD45 gene. A: Schematic diagram illustrating the co-transfection of sgRNA-CD45-CAG-3 and its hsgRNA-CD45-CAG-3 with tBE-V5-mA3 and nCas9. B: Editing efficiency induced by tBE-V5-mA3 with indicated pair of sgRNA/hsgRNA at indicated sites.

FIG. 38: Editing efficiencies induced by tBE with the pair of sgRNA-CD45-CAG-4 and its hsgRNA targeting human CD45 gene. A: Schematic diagram illustrating the co-transfection of sgRNA-CD45-CAG-4 and its hsgRNA-CD45-CAG-4 with tBE-V5-mA3 and nCas9. B: Editing efficiency induced by tBE-V5-mA3 with indicated pair of sgRNA/hsgRNA at indicated sites.

FIG. 39: Editing efficiencies induced by tBE with the pair of sgRNA-CD45-CAG-5 and its hsgRNA targeting human CD45 gene. A: Schematic diagram illustrating the co-transfection of sgRNA-CD45-CAG-5 and its hsgRNA-CD45-CAG-5 with tBE-V5-mA3 and nCas9. B: Editing efficiency induced by tBE-V5-mA3 with indicated pair of sgRNA/hsgRNA at indicated sites.

FIG. 40: Editing efficiencies induced by tBE with the pair of sgRNA-CD45-CGA-6 and its hsgRNA targeting human CD45 gene. A: Schematic diagram illustrating the co-transfection of sgRNA-CD45-CGA-6 and its hsgRNA-CD45-CGA-6 with tBE-V5-mA3 and nCas9. B: Editing efficiency induced by tBE-V5-mA3 with indicated pair of sgRNA/hsgRNA at indicated sites.

FIG. 41: Editing efficiencies induced by tBE with the pair of sgRNA-CD45-CAG/CGA-7 and its hsgRNA targeting human CD45 gene. A: Schematic diagram illustrating the co-transfection of sgRNA-CD45-CAG/CGA-7 and its hsgRNA-CD45-CAG/CGA-7 with tBE-V5-mA3 and nCas9. B: Editing efficiency induced by tBE-V5-mA3 with indicated pair of sgRNA/hsgRNA at indicated sites.

FIG. 42: Editing efficiencies induced by tBE with the pair of sgRNA-CD45-TGG-8 and its hsgRNA targeting human CD45 gene. A: Schematic diagram illustrating the co-transfection of sgRNA-CD45-TGG-8 and its hsgRNA-CD45-TGG-8 with tBE-V5-mA3 and nCas9. B: Editing efficiency induced by tBE-V5-mA3 with indicated pair of sgRNA/hsgRNA at indicated sites.

FIG. 43: Editing efficiencies induced by tBE with the pair of sgRNA-CD45-TGG-9 and its hsgRNA targeting human CD45 gene. A: Schematic diagram illustrating the co-transfection of sgRNA-CD45-TGG-9 and its hsgRNA-CD45-TGG-9 with tBE-V5-mA3 and nCas9. B: Editing efficiency induced by tBE-V5-mA3 with indicated pair of sgRNA/hsgRNA at indicated sites.

FIG. 44: Editing efficiencies induced by tBE with the pairs of sgRNA-CD45-AG-10 and its different hsgRNAs targeting human CD45 gene. A: Schematic diagram illustrating the co-transfection of sgRNA-CD45-AG-10 and its different hsgRNA-CD45-AG-10s with tBE-V5-mA3 and nCas9. B: Editing efficiency induced by tBE-V5-mA3 with indicated pairs of sgRNA/hsgRNA at indicated sites.

FIG. 45: Editing efficiencies induced by tBE with the pairs of sgRNA-CD45-AG-11 and its different hsgRNAs targeting human CD45 gene. A: Schematic diagram illustrating the co-transfection of sgRNA-CD45-AG-11 and its different hsgRNA-CD45-AG-11s with tBE-V5-mA3 and nCas9. B: Editing efficiency induced by tBE-V5-mA3 with indicated pairs of sgRNA/hsgRNA at indicated sites.

FIG. 46: Editing efficiencies induced by tBE with the pairs of sgRNA-CD45-GU-12 and its different hsgRNAs targeting human CD45 gene. A: Schematic diagram illustrating the co-transfection of sgRNA-CD45-GU-12 and its different hsgRNA-CD45-GU-12s with tBE-V5-mA3 and nCas9. B: Editing efficiency induced by tBE-V5-mA3 with indicated pairs of sgRNA/hsgRNA at indicated sites.

FIG. 47: Editing efficiencies induced by tBE with the pair of sgRNA-CD45-GU-13 and its hsgRNA targeting human CD45 gene. A: Schematic diagram illustrating the co-transfection of sgRNA-CD45-GU-13 and its hsgRNA-CD45-GU-13 with tBE-V5-mA3 and nCas9. B: Editing efficiency induced by tBE-V5-mA3 with indicated pair of sgRNA/hsgRNA at indicated sites.

FIG. 48: Editing efficiencies induced by tBE with the pairs of sgRNA-CLL1-CAG-1 and its different hsgRNAs targeting human CLL1 gene. A: Schematic diagram illustrating the co-transfection of sgRNA-CLL1-CAG-1 and its different hsgRNA-CLL1-CAG-1s with tBE-V5-mA3 and nCas9. B: Editing efficiency induced by tBE-V5-mA3 with indicated pairs of sgRNA/hsgRNA at indicated sites.

FIG. 49: Editing efficiencies induced by tBE with the pairs of sgRNA-CLL1-TGG-2 and its different hsgRNAs targeting human CLL1 gene. A: Schematic diagram illustrating the co-transfection of sgRNA-CLL1-TGG-2 and its different hsgRNA-CLL1-TGG-2s with tBE-V5-mA3 and nCas9. B: Editing efficiency induced by tBE-V5-mA3 with indicated pairs of sgRNA/hsgRNA at indicated sites.

FIG. 50: Editing efficiencies induced by tBE with the pairs of sgRNA-CLL1-TGG-3 and its different hsgRNAs targeting human CLL1 gene. A: Schematic diagram illustrating the co-transfection of sgRNA-CLL1-TGG-3 and its different hsgRNA-CLL1-TGG-3s with tBE-V5-mA3 and nCas9. B: Editing efficiency induced by tBE-V5-mA3 with indicated pairs of sgRNA/hsgRNA at indicated sites.

FIG. 51: Comparison of on-target editing and off-target mutation frequencies induced by tBE-V5-mA3, hA3A-BE3, BE4 and BE4max at hCD33-EXON2-CAG-4 site and a sgRNA-dependent OT site.

FIG. 52: Comparison of on-target editing and off-target mutation frequencies induced by tBE-V5-mA3, hA3A-BE3 and BE4 at hCD33-EXON2-TGG-9 site and a sgRNA-dependent OT site.

FIG. 53: Comparison of on-target editing and off-target mutation frequencies induced by tBE-V5-mA3, hA3A-BE3, BE4 and BE4max at hCD33-EXON2-TGG-10 site and three sgRNA-dependent OT sites.

FIG. 54: Comparison of on-target editing and off-target mutation frequencies induced by tBE-V5-mA3, hA3A-BE3 and BE4 at hCD33-EXON3-TGG-11 site and two sgRNA-dependent OT sites.

FIG. 55: Comparison of on-target editing and off-target mutation frequencies induced by tBE-V5-mA3, hA3A-BE3, BE4 and BE4max at hCD33-EXON4-AG-15 site and two sgRNA-dependent OT sites.

FIG. 56: Comparison of on-target editing and off-target mutation frequencies induced by tBE-V5-mA3, hA3A-BE3 and BE4 at hCD123-EXON5-CAG-3 site and two sgRNA-dependent OT sites.

FIG. 57: Comparison of on-target editing and off-target mutation frequencies induced by tBE-V5-mA3, hA3A-BE3, BE4 and BE4max at hCD123-EXON5-CGA-6 site and a sgRNA-dependent OT site.

FIG. 58: Comparison of on-target editing and off-target mutation frequencies induced by tBE-V5-mA3, hA3A-BE3 and BE4 at hCD123-EXON1-TGG-7 site and a sgRNA-dependent OT site.

FIG. 59: Comparison of on-target editing and off-target mutation frequencies induced by tBE-V5-mA3, hA3A-BE3 and BE4 at hCD47-EXON6-AG-3 site and two sgRNA-dependent OT sites.

FIG. 60: Comparison of on-target editing and off-target mutation frequencies induced by tBE-V5-mA3, hA3A-BE3 and BE4 at hCD45-EXON11-CAA-1 site and two sgRNA-dependent OT sites.

FIG. 61: Comparison of on-target editing and off-target mutation frequencies induced by tBE-V5-mA3, hA3A-BE3 and BE4 at hCD45-EXON13-TGG-8 site and a sgRNA-dependent OT site.

FIG. 62: Comparison of on-target editing and off-target mutation frequencies induced by tBE-V5-mA3, hA3A-BE3 and BE4 at hCLL1-EXON4-TGG-2 site and two sgRNA-dependent OT sites.

FIG. 63: Comparison of on-target editing and off-target mutation frequencies induced by tBE-V5-mA3, hA3A-BE3 and BE4 at hCLL1-EXON4-TGG-3 site and two sgRNA-dependent OT sites.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### Regeneration of Surface Antigen-Negative Cells

As provided, the expression of surface antigens on a normal cell can cause serious toxicities in cancer patients that are treated with therapies targeting such antigens. Generating non-cancerous cells not targeted by such therapies can help reduce the toxicities. The instant inventors have developed a new base editing system, transformer base editor (tBE), which can specifically edit cytosine in target regions with no observable off-target mutations. The tBE technology can be suitably employed to generate surface antigen-negative cells.

The tBE system is composed of a cytidine deaminase inhibitor (dCDI) and split-TEV system. tBE remains inactive at off-target sites with a cleavable fusion of dCDI domain, thus eliminating unintended mutations. Only when binding at on-target sites, tBE is transformed to cleave off the dCDI domain and catalyzes targeted deamination for precise editing. More specifically, tBE uses a sgRNA (normally 20 nt) to bind at the target genomic site and a helper sgRNA (hsgRNA, normally 10 or 20 nt) to bind at a nearby region upstream to the target genomic site. The binding of two sgRNAs can guide the components of the tBE system to correctly assemble at the target genomic site for base editing.

As demonstrated in the accompanying examples, the tBE technology can be used to perform highly specific and efficient base editing in living organisms and enables potential clinical applications, e.g., inducing a premature stop codon to repress CD33 or CD123 protein expression or breaking the GU-AG rule to disrupt splicing sites. Generation of CD33 or CD123-negative cells can widen the targeted therapeutic index of a variety of treatment modalities in AML, including monoclonal antibodies, antibody-drug conjugates, and bi-specific T cell engagers.

In accordance with one embodiment of the present disclosure, therefore, provided is a base editing system, or one or more polynucleotides encoding the base editing system, useful for reducing the biological activity of a cell surface antigen in a cell.

In some embodiments, the base editing system includes a CRISPR-associated (Cas) protein, a nucleobase deaminase, a single-guide RNA (sgRNA)/helper single-guide RNA (hsgRNA) pair targeting the cell surface antigen to introduce a premature stop codon or disrupt a splicing site.

“Guide RNAs” are non-coding short RNA sequences which bind to the complementary target DNA sequences. A guide RNA first binds to the Cas enzyme and the gRNA sequence guides the complex via pairing to a specific location on the DNA, where Cas performs its endonuclease activity by cutting the target DNA strand. A “single guide RNA,” frequently simply referred to as “guide RNA”, refers to synthetic or expressed single guide RNA (sgRNA) that consists of both the crRNA and tracrRNA as a single construct. The tracrRNA portion is responsible for Cas endonuclease activity and the crRNA portion binds to the target specific DNA region. Therefore, the trans activating RNA (tracrRNA, or scaffold region) and crRNA are two key components and are joined by tetraloop which results in formation of sgRNA. Guide RNA targets the complementary sequences by simple Watson-Crick base pairing. TracrRNA are base pairs having a stemloop structure in itself and attaches to the endonuclease enzyme. crRNA includes a spacer, complementary to the target sequence, flanked region due to repeat sequences.

In one embodiment, the cell surface antigen is CD33. Example sgRNA/hsgRNA pairs are shown in Tables 1A-1C. In Table 1A, each sgRNA can be paired with any one of the corresponding hsgRNA, and they can be used to make C-to-T editing to create a stop codon at a CAG or CAA codon in the CD33 gene. In Table 1B, each sgRNA can be paired with any one of the corresponding hsgRNA, and they can be used to make G-to-A editing to create a stop codon at a TGG codon in the CD33 gene. In Table 1C, each sgRNA can be paired with any one of the corresponding hsgRNA, and they can be used to make G-to-A editing to disrupt a GU-AG splicing site in the CD33 gene. For each hsgRNA, the tables provide a 20-nt sequence and a shorter 10-nt version within the 20-nt sequence, either of which is sufficient for the editing.

In some embodiments, the sgRNA includes the nucleic acid sequence of SEQ ID NO:1, and the hsgRNA includes any of the nucleic acid sequences of SEQ ID NO:51 to 62. In some embodiments, the sgRNA includes the nucleic acid sequence of SEQ ID NO:2, and the hsgRNA includes any of the nucleic acid sequences of SEQ ID NO:63-74. In some embodiments, the sgRNA includes the nucleic acid sequence of SEQ ID NO:3, and the hsgRNA includes any of the nucleic acid sequences of SEQ ID NO:75-86. In some embodiments, the sgRNA includes the nucleic acid sequence of SEQ ID NO:4, and the hsgRNA includes any of the nucleic acid sequences of SEQ ID NO:87-98. In some embodiments, the sgRNA includes the nucleic acid sequence of SEQ ID NO:5, and the hsgRNA includes any of the nucleic acid sequences of SEQ ID NO:99-108. In some embodiments, the sgRNA includes the nucleic acid sequence of SEQ ID NO:6, and the hsgRNA includes any of the nucleic acid sequences of SEQ ID NO:109-114.

In some embodiments, the sgRNA includes the nucleic acid sequence of SEQ ID NO:7, and the hsgRNA includes any of the nucleic acid sequences of SEQ ID NO:115-124. In some embodiments, the sgRNA includes the nucleic acid sequence of SEQ ID NO:8, and the hsgRNA includes any of the nucleic acid sequences of SEQ ID NO:125-130. In some embodiments, the sgRNA includes the nucleic acid sequence of SEQ ID NO:9, and the hsgRNA includes any of the nucleic acid sequences of SEQ ID NO:131-136. In some embodiments, the sgRNA includes the nucleic acid sequence of SEQ ID NO:10, and the hsgRNA includes any of the nucleic acid sequences of SEQ ID NO:137-154. In some embodiments, the sgRNA includes the nucleic acid sequence of SEQ ID NO:11, and the hsgRNA includes any of the nucleic acid sequences of SEQ ID NO:155-168.

In some embodiments, the sgRNA includes the nucleic acid sequence of SEQ ID NO:12, and the hsgRNA includes any of the nucleic acid sequences of SEQ ID NO:169-178. In some embodiments, the sgRNA includes the nucleic acid sequence of SEQ ID NO:13, and the hsgRNA includes any of the nucleic acid sequences of SEQ ID NO:179-194. In some embodiments, the sgRNA includes the nucleic acid sequence of SEQ ID NO:14, and the hsgRNA includes any of the nucleic acid sequences of SEQ ID NO:195-198. In some embodiments, the sgRNA includes the nucleic acid sequence of SEQ ID NO:15, and the hsgRNA includes any of the nucleic acid sequences of SEQ ID NO:199-208. In some embodiments, the sgRNA includes the nucleic acid sequence of SEQ ID NO:16, and the hsgRNA includes any of the nucleic acid sequences of SEQ ID NO:209-210. In some embodiments, the sgRNA includes the nucleic acid sequence of SEQ ID NO:17, and the hsgRNA includes any of the nucleic acid sequences of SEQ ID NO:211-216. In some embodiments, the sgRNA includes the nucleic acid sequence of SEQ ID NO:18, and the hsgRNA includes any of the nucleic acid sequences of SEQ ID NO:217-224.

In Table 1D, each sgRNA can be paired with any one of the corresponding hsgRNA, and they can be used to make C-to-T editing to create a stop codon at a CGA, CAG or CAA codon in the CD123 gene. In Table 1E, each sgRNA can be paired with any one of the corresponding hsgRNA, and they can be used to make G-to-A editing to create a stop codon at a TGG codon in the CD123 gene. In Table 1F, each sgRNA can be paired with any one of the corresponding hsgRNA, and they can be used to make G-to-A editing to disrupt a GU-AG splicing site in the CD123 gene. For each hsgRNA, the tables provide a 20-nt sequence and a shorter 10-nt version within the 20-nt sequence, either of which is sufficient for the editing.

In some embodiments, the sgRNA includes the nucleic acid sequence of SEQ ID NO:19, and the hsgRNA includes any of the nucleic acid sequences of SEQ ID NO:225-230. In some embodiments, the sgRNA includes the nucleic acid sequence of SEQ ID NO:20, and the hsgRNA includes any of the nucleic acid sequences of SEQ ID NO:231-236. In some embodiments, the sgRNA includes the nucleic acid sequence of SEQ ID NO:21, and the hsgRNA includes any of the nucleic acid sequences of SEQ ID NO:237-240. In some embodiments, the sgRNA includes the nucleic acid sequence of SEQ ID NO:22, and the hsgRNA includes any of the nucleic acid sequences of SEQ ID NO:241-244. In some embodiments, the sgRNA includes the nucleic acid sequence of SEQ ID NO:23, and the hsgRNA includes any of the nucleic acid sequences of SEQ ID NO:245-254. In some embodiments, the sgRNA includes the nucleic acid sequence of SEQ ID NO:24, and the hsgRNA includes any of the nucleic acid sequences of SEQ ID NO:255-260.

In some embodiments, the sgRNA includes the nucleic acid sequence of SEQ ID NO:25, and the hsgRNA includes any of the nucleic acid sequences of SEQ ID NO:261-270. In some embodiments, the sgRNA includes the nucleic acid sequence of SEQ ID NO:26, and the hsgRNA includes any of the nucleic acid sequences of SEQ ID NO:271-274.

In some embodiments, the sgRNA includes the nucleic acid sequence of SEQ ID NO:27, and the hsgRNA includes any of the nucleic acid sequences of SEQ ID NO:275-278. In some embodiments, the sgRNA includes the nucleic acid sequence of SEQ ID NO:28, and the hsgRNA includes any of the nucleic acid sequences of SEQ ID NO:279-284. In some embodiments, the sgRNA includes the nucleic acid sequence of SEQ ID NO:29, and the hsgRNA includes any of the nucleic acid sequences of SEQ ID NO:285-286. In some embodiments, the sgRNA includes the nucleic acid sequence of SEQ ID NO:30, and the hsgRNA includes any of the nucleic acid sequences of SEQ ID NO:287-290. In some embodiments, the sgRNA includes the nucleic acid sequence of SEQ ID NO:31, and the hsgRNA includes any of the nucleic acid sequences of SEQ ID NO:291-300.

In Table 1G, each sgRNA can be paired with any one of the corresponding hsgRNA, and they can be used to make G-to-A editing to create a stop codon at a TGG codon in the CD47 gene. In Table 1H, each sgRNA can be paired with any one of the corresponding hsgRNA, and they can be used to make G-to-A editing to disrupt a GU-AG splicing site in the CD47 gene. For each hsgRNA, the tables provide a 20-nt sequence and a shorter 10-nt version within the 20-nt sequence, either of which is sufficient for the editing.

In some embodiments, the sgRNA includes the nucleic acid sequence of SEQ ID NO:32, and the hsgRNA includes any of the nucleic acid sequences of SEQ ID NO:301-306.

In some embodiments, the sgRNA includes the nucleic acid sequence of SEQ ID NO:33, and the hsgRNA includes any of the nucleic acid sequences of SEQ ID NO:307-310. In some embodiments, the sgRNA includes the nucleic acid sequence of SEQ ID NO:34, and the hsgRNA includes any of the nucleic acid sequences of SEQ ID NO:311-318.

In Table 1I, each sgRNA can be paired with any one of the corresponding hsgRNA, and they can be used to make C-to-T editing to create a stop codon at a CGA, CAG or CAA codon in the CD45 gene. In Table 1J, each sgRNA can be paired with any one of the corresponding hsgRNA, and they can be used to make G-to-A editing to create a stop codon at a TGG codon in the CD45 gene. In Table 1K, each sgRNA can be paired with any one of the corresponding hsgRNA, and they can be used to make G-to-A editing to disrupt a GU-AG splicing site in the CD45 gene. For each hsgRNA, the tables provide a 20-nt sequence and a shorter 10-nt version within the 20-nt sequence, either of which is sufficient for the editing.

In some embodiments, the sgRNA includes the nucleic acid sequence of SEQ ID NO:35, and the hsgRNA includes any of the nucleic acid sequences of SEQ ID NO:319-322. In some embodiments, the sgRNA includes the nucleic acid sequence of SEQ ID NO:36, and the hsgRNA includes any of the nucleic acid sequences of SEQ ID NO:323-324. In some embodiments, the sgRNA includes the nucleic acid sequence of SEQ ID NO:37, and the hsgRNA includes any of the nucleic acid sequences of SEQ ID NO:325-326. In some embodiments, the sgRNA includes the nucleic acid sequence of SEQ ID NO:38, and the hsgRNA includes any of the nucleic acid sequences of SEQ ID NO:327-328. In some embodiments, the sgRNA includes the nucleic acid sequence of SEQ ID NO:39, and the hsgRNA includes any of the nucleic acid sequences of SEQ ID NO:329-332. In some embodiments, the sgRNA includes the nucleic acid sequence of SEQ ID NO:40, and the hsgRNA includes any of the nucleic acid sequences of SEQ ID NO:333-338. In some embodiments, the sgRNA includes the nucleic acid sequence of SEQ ID NO:41, and the hsgRNA includes any of the nucleic acid sequences of SEQ ID NO:339-340.

In some embodiments, the sgRNA includes the nucleic acid sequence of SEQ ID NO:42, and the hsgRNA includes any of the nucleic acid sequences of SEQ ID NO:341-342. In some embodiments, the sgRNA includes the nucleic acid sequence of SEQ ID NO:43, and the hsgRNA includes any of the nucleic acid sequences of SEQ ID NO:343-344.

In some embodiments, the sgRNA includes the nucleic acid sequence of SEQ ID NO:44, and the hsgRNA includes any of the nucleic acid sequences of SEQ ID NO:345-354. In some embodiments, the sgRNA includes the nucleic acid sequence of SEQ ID NO:45, and the hsgRNA includes any of the nucleic acid sequences of SEQ ID NO:355-372. In some embodiments, the sgRNA includes the nucleic acid sequence of SEQ ID NO:46, and the hsgRNA includes any of the nucleic acid sequences of SEQ ID NO:373-382. In some embodiments, the sgRNA includes the nucleic acid sequence of SEQ ID NO:47, and the hsgRNA includes any of the nucleic acid sequences of SEQ ID NO:383-384.

In Table 1L, each sgRNA can be paired with any one of the corresponding hsgRNA, and they can be used to make C-to-T editing to create a stop codon at a CAG codon in the CLL1 gene. In Table 1M, each sgRNA can be paired with any one of the corresponding hsgRNA, and they can be used to make G-to-A editing to create a stop codon at a TGG codon in the CLL1 gene. For each hsgRNA, the tables provide a 20-nt sequence and a shorter 10-nt version within the 20-nt sequence, either of which is sufficient for the editing.

In some embodiments, the sgRNA includes the nucleic acid sequence of SEQ ID NO:48, and the hsgRNA includes any of the nucleic acid sequences of SEQ ID NO:385-392.

In some embodiments, the sgRNA includes the nucleic acid sequence of SEQ ID NO:49, and the hsgRNA includes any of the nucleic acid sequences of SEQ ID NO:393-398. In some embodiments, the sgRNA includes the nucleic acid sequence of SEQ ID NO:50, and the hsgRNA includes any of the nucleic acid sequences of SEQ ID NO:399-404.

The term “nucleobase deaminase” as used herein, refers to a group of enzymes that catalyze the hydrolytic deamination of nucleobases such as cytidine, deoxycytidine, adenosine and deoxyadenosine. Non-limiting examples of nucleobase deaminases include cytidine deaminases and adenosine deaminases.

“Cytidine deaminase” refers to enzymes that catalyze the irreversible hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

Various mutants of the APOBEC proteins are also known that have bring about different editing characteristics for base editors. For instance, for human APOBEC3A, certain mutants (e.g., W98Y, Y130F, Y132D, W104A, D131Y and P134Y) even outperform the wildtype human APOBEC3A in terms of editing efficiency or editing window. Accordingly, the term APOBEC and each of its family member also encompasses variants and mutants that have certain level (e.g., 70%, 75%, 80%, 85%, 90%, 95%, 98%, 99%) of sequence identity to the corresponding wildtype APOBEC protein or the catalytic domain and retain the cytidine deaminating activity. The variants and mutants can be derived with amino acid additions, deletions and/or substitutions. Such substitutions, in some embodiments, are conservative substitutions.

“Adenosine deaminase”, also known as adenosine aminohydrolase, or ADA, is an enzyme (EC 3.5.4.4) involved in purine metabolism. It is needed for the breakdown of adenosine from food and for the turnover of nucleic acids in tissues.

Non-limiting examples of adenosine deaminases include tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), adenosine deaminase RNA specific (ADAR) and adenosine deaminase RNA specific B1 (ADARB1).

Some of the nucleobase deaminases have a single, catalytic domain, while others also have other domains, such as an inhibitory domain as currently discovered by the instant inventors.

In some embodiments, therefore, the first fragment only includes the catalytic domain, such as mA3-CDA1, hA3F-CDA2 and hA3B-CDA2. In some embodiments, the first fragment includes at least a catalytic core of the catalytic domain.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

In some embodiments, the base editing system further includes a nucleobase deaminase inhibitor fused to the nucleobase deaminase. A “nucleobase deaminase inhibitor,” accordingly, refers to a protein or a protein domain that inhibits the deaminase activity of a nucleobase deaminase. In some embodiments, the second fragment includes at least an inhibitory core of the inhibitory protein/domain.

Two example nucleobase deaminase inhibitors are mA3-CDA2, hA3F-CDA1 and hA3B-CDA1, which are the inhibitory domains of the corresponding nucleobase deaminases. Additional nucleobase deaminase inhibitors have been identified in the protein databases as homologues of mA3-CDA2, hA3F-CDA1 and hA3B-CDA1 (see, e.g., WO2020156575A1). Their biological equivalents (e.g., having at least about 80%, 85%, 90%, 95%, 97%, 98%, 99%, 99.5% sequence identity, or having one, two, or three amino acid addition/deletion/substitution, and having nucleobase deaminase inhibitor activity) can also be prepared with known methods in the art, such as conservative amino acid substitutions.

When the nucleobase deaminase inhibitor is included, it is fused to the nucleobase deaminase but is separated by a protease cleavage site. In some embodiments, the base editing system further includes the protease that is capable of cleaving the protease cleavage site.

The protease cleavage site can be any known protease cleavage site (peptide) for any proteases. Non-limiting examples of proteases include TEV protease, TuMV protease, PPV protease, PVY protease, ZIKV protease and WNV protease.

In some embodiments, the protease cleavage site is a self-cleaving peptide, such as the 2A peptides. “2A peptides” are 18-22 amino-acid-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified.

In some embodiments, the protease cleavage site is a cleavage site for the TEV protease. In some embodiments, the TEV protease provided in the base editing system includes two separate fragments, each of which on its own is not active. However, in the presence of the remaining fragment of the TEV protease, they will be able to execute the cleavage. Such an arrangement provides additional control and flexible of the base editing capabilities. The TEV fragments may be the TEV N-terminal domain or the TEV C-terminal domain.

Such fusion proteins may include other fragments, such as uracil DNA glycosylase inhibitor (UGI) and nuclear localization sequences (NLS). A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A non-limiting example of NLS is the internal SV40 nuclear localization sequence (iNLS).

The “Uracil Glycosylase Inhibitor” (UGI), which can be prepared from Bacillus subtilis bacteriophage PBS1, is a small protein (9.5 kDa) which inhibits E. coli uracil-DNA glycosylase (UDG) as well as UDG from other species. Inhibition of UDG occurs by reversible protein binding with a 1:1 UDG:UGI stoichiometry. UGI is capable of dissociating UDG-DNA complexes.

In some embodiments, a peptide linker is optionally provided between each of the fragments in the fusion protein. In some embodiments, the peptide linker has from 1 to 100 amino acid residues (or 3-20, 4-15, without limitation). In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine.

The disclosed base editing system can be used to engineer a target cell. The editing approach can disrupt the expression of a normal cell surface antigen in the target cell, which can be carried out in vitro, ex vivo, or in vivo. The engineered target cell would be resistant to therapies designed to destroy cells, such as tumor cells, that express such surface antigens. For instance, if a patient receiving an anti-CD33 immunotherapy suffers from dysfunction of CD33-expressing myeloid cells, the present editing technology can lead to production of myeloid cells not targeted by the anti-CD33 therapy and thus restore the function of regular myeloid cells.

In some embodiments, each component of the base editing system can be introduced to the target cell individually, or in combination. For instance, a fusion protein may be packaged into nanoparticle such as liposome. In another example, a guide RNA and a protein may be combined into a complex for introduction.

In some embodiments, some or all of the components of the base editing system can be introduced as one or more polynucleotides encoding them. These polynucleotides may be constructed as plasmids or viral vectors, without limitation.

In an example ex vivo approach, CD34+ hematopoietic stem and progenitor cells (HSPCs) can be collected from a patient. The HSPCs can then be edited with the disclosed gene editing technology, along with the designed sgRNA/hsgRNA, to produce edited cells. DNA sequencing can be used to evaluate the percentage of allelic editing at the on-target site. The edited cells can be injected back to the patient which can help reduce surface antigen-targeted therapy-mediated toxicities. Prior to infusion of the edited cells, the patient can be given a pharmacokinetically adjusted busulfan myeloablation. The edited cells can be administered through intravenous infusion.

Cells, genomic sequences, mRNA sequences, and proteins that can be prepared by the instant base editing technologies and designed sgRNA/hsgRNA sequences are also provided, in some embodiments.

In some embodiments, the genomic sequence originally encodes the human CD33 protein, but has been edited by the instant base editing system such that the normal expression of the CD33 protein is disrupted. The disrupted expression, in some embodiments, is due to introduction of a premature stop codon, a frame shift mutation or an altered splicing site. In some embodiments, a mutated mRNA encoded by the edited genomic sequence is provided. In some embodiments, a mutated CD33 protein encoded by the edited genomic sequence is provided. In some embodiments, a cell that contains the genomic sequence, the mRNA or the protein is provided.

Likewise, in some embodiments, a genomic sequence that encode a disrupted CD123, CD47, CD45 or CLL1 protein is also provided. In some embodiments, a mutated mRNA encoded by the edited genomic sequence is provided. In some embodiments, a mutated CD123, CD47, CD45 or CLL1 protein encoded by the edited genomic sequence is provided. In some embodiments, a cell that contains the genomic sequence, the mRNA or the protein is provided.

### Examples

### Example 1. Gene Editing for Disrupting the Expression of Genes

This example employed a transformer Base Editor (tBE) to disrupt certain genes which can be useful for treating acute myeloid leukemia (AML).

The transformer Base Editor (tBE) a new base editor that specifically edits cytosine in a target region with no observable off-target mutations. In the tBE system, a cytidine deaminase is fused with a nucleobase deaminase inhibitor to inhibit the activity of the nucleobase deaminase until the tBE complex is assembled at the target genomic site. In some instances, the tBE employs a sgRNA to bind at the target genomic site and a helper sgRNA to bind at a nearby region upstream to the target genomic site. The binding of two sgRNAs can guide the components of tBE to correctly assemble at the target genomic site for efficient base editing. Upon such assembly, a protease in the tBE system is activated, capable of cleaving the nucleobase deaminase inhibitor off from the nucleobase deaminase, which becomes activated.

To apply the tBE system to generate stop codons or disrupt splicing site in CD33 gene, this example designed 87 pairs of sgRNA/hsgRNAs that target the CD33 gene (Table 1A-1M).

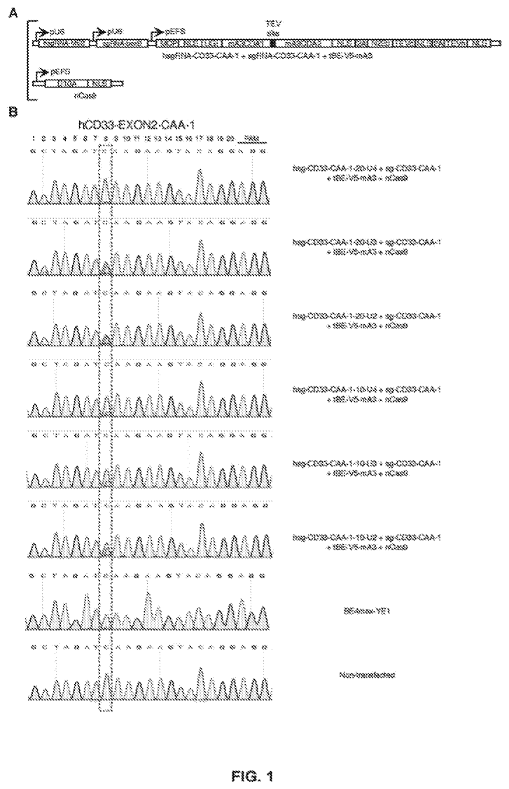

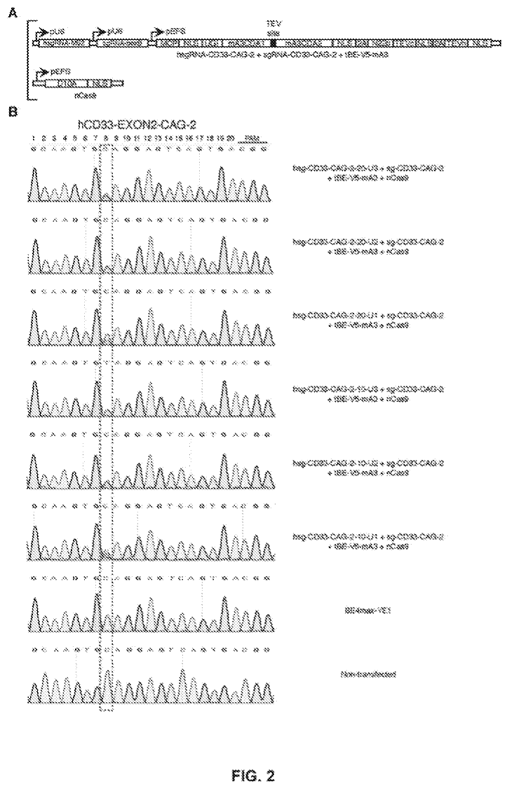

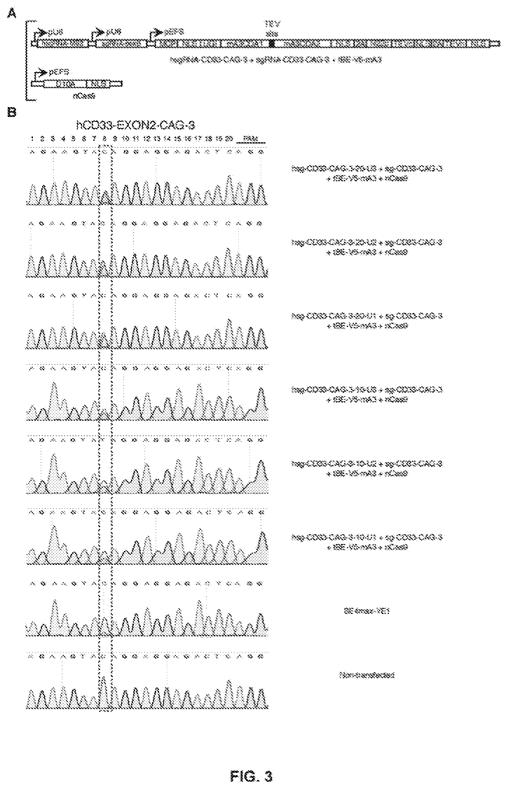

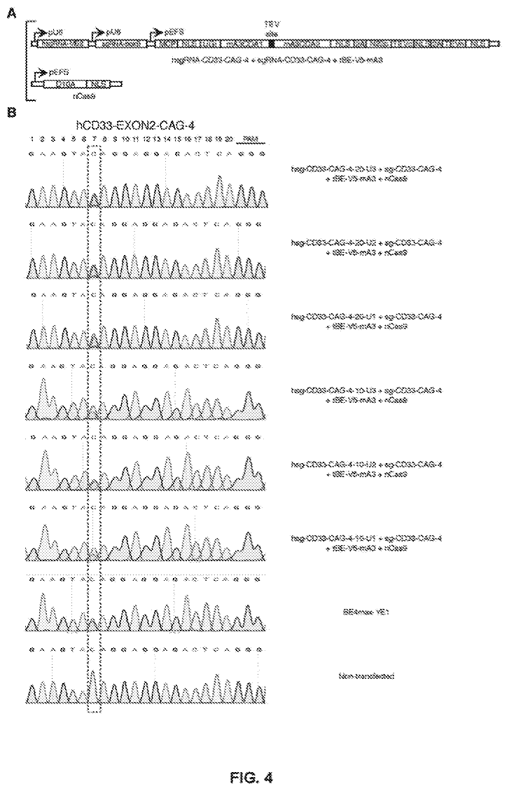

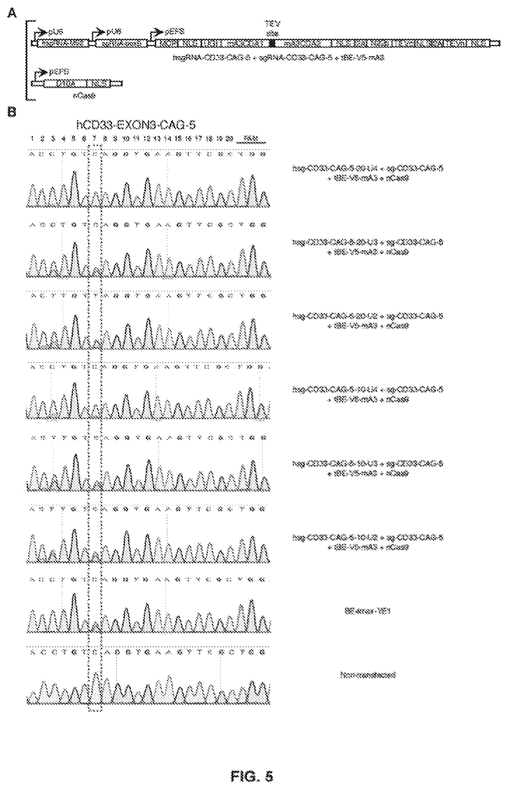

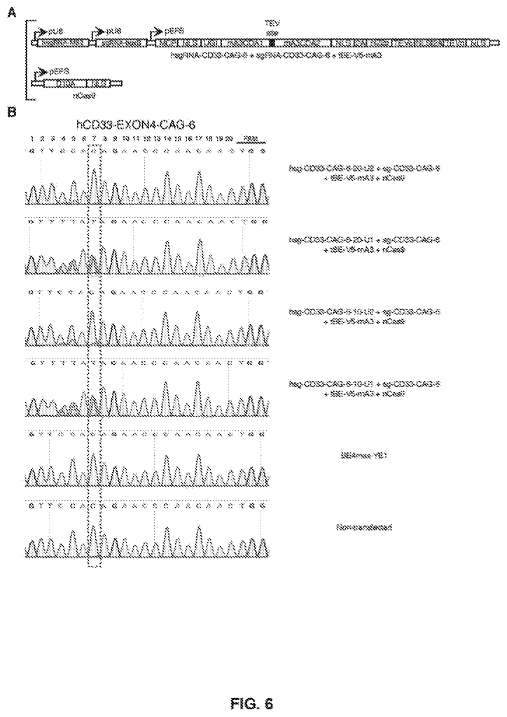

First, this example used tBE to induce C-to-T base editing in the codons of CAG (Gln) and CAA (Gln) in CD33 genes to create TAG and TAA stop codon (Table 1, FIG. 1-6). For comparison, we co-transfected the sgRNAs of sgRNA/hsgRNA pairs with a previously reported CBE variant, BE4max-YE1. We extracted genomic DNA 72 hours after transfecting plasmids into the cells, and compared the C-to-T editing efficiencies of these BEs at target sites. From Sanger sequencing results, we found that both tBE and BE4max-YE1 induced gene editing in the CD33 gene. tBE, however induced higher base editing efficiencies than BE4max-YE1 at most target sites, such as the target sites for hsgRNA-CD33-CAA-1-20-U2/sgRNA-CD33-CAA-1 (FIG. 1B) and hsgRNA-CD33-CAG-2-20-U2/sgRNA-CD33-CAG-2 (FIG. 2B). These results demonstrate that tBE can perform highly efficient base editing to generate stop codons in the CD33 gene.

TABLE 1A
sgRNA and hsgRNA Sequences
C-to-T editing to create stop codon at CAA or CAG in CD33

| | | | SEQ | | SEQ |
|---|---|---|---|---|---|
| | | | ID | | ID |
| Type | Name | 20 nt | NO: | 10 nt | NO: |
| sgRNA | CD33-CAA-1 | gcuagaucaagaaguacagg | 1 | — | |
| hsgRNA | CD33-CAA-1-U1 | uauauccagggacucuccag | 51 | gacucuccag | 57 |
| | CD33-CAA-1-U2 | ggaaggagccauuauaucca | 52 | auuauaucca | 58 |
| | CD33-CAA-1-U3 | caugguuacugguuccggga | 53 | gguuccggga | 59 |
| | CD33-CAA-1-U4 | aguucaugguuacugguucc | 54 | uacugguucc | 60 |
| | CD33-CAA-1-U5 | acuccccaguucaugguuac | 55 | ucaugguuac | 61 |
| | CD33-CAA-1-U6 | gacaagaacuccccaguuca | 56 | ccccaguuca | 62 |
| sgRNA | CD33-CAG-2 | gcaagugcaggagucaguga | 2 | — | |
| hsgRNA | CD33-CAG-2-U1 | ccccacagggguccuggcua | 63 | guccuggcua | 69 |
| | CD33-CAG-2-U2 | ucguuuccccacaggggucc | 64 | acaggggucc | 70 |
| | CD33-CAG-2-U3 | ugacccucguuuccccacag | 65 | uuccccacag | 71 |
| | CD33-CAG-2-U4 | gcugacccucguuuccccac | 66 | guuuccccac | 72 |
| | CD33-CAG-2-U5 | gggagagggguugucgggcu | 67 | uugucgggcu | 73 |
| | CD33-CAG-2-U6 | cuguggggagagggguuguc | 68 | agggguuguc | 74 |
| sgRNA | CD33-CAG-3 | agaaguacaggaggagacuc | 3 | — | |
| hsgRNA | CD33-CAG-3-U1 | uauauccagggacucuccag | 75 | gacucuccag | 81 |
| | CD33-CAG-3-U2 | ggaaggagccauuauaucca | 76 | auuauaucca | 82 |
| | CD33-CAG-3-U3 | caugguuacugguuccggga | 77 | gguuccggga | 83 |
| | CD33-CAG-3-U4 | aguucaugguuacugguucc | 78 | uacugguucc | 84 |
| | CD33-CAG-3-U5 | acuccccaguucaugguuac | 79 | ucaugguuac | 85 |
| | CD33-CAG-3-U6 | gacaagaacuccccaguuca | 80 | ccccaguuca | 86 |
| sgRNA | CD33-CAG-4 | gaaguacaggaggagacuca | 4 | — | |
| hsgRNA | CD33-CAG-4-U1 | uauauccagggacucuccag | 87 | gacucuccag | 93 |
| | CD33-CAG-4-U2 | ggaaggagccauuauaucca | 88 | auuauaucca | 94 |
| | CD33-CAG-4-U3 | caugguuacugguuccggga | 89 | gguuccggga | 95 |
| | CD33-CAG-4-U4 | aguucaugguuacugguucc | 90 | uacugguucc | 96 |
| | CD33-CAG-4-U5 | acuccccaguucaugguuac | 91 | ucaugguuac | 97 |
| | CD33-CAG-4-U6 | gacaagaacuccccaguuca | 92 | ccccaguuca | 98 |
| sgRNA | CD33-CAG-5 | accugucaggugaaguucgc | 5 | — | |
| hsgRNA | CD33-CAG-5-U1 | accccacggccccaggacca | 99 | cccaggacca | 104 |
| | CD33-CAG-5-U2 | cauaaucaccccacggcccc | 100 | ccacggcccc | 105 |
| | CD33-CAG-5-U3 | cggugcucauaaucacccca | 101 | aaucacccca | 106 |
| | CD33-CAG-5-U4 | ccccaggacuacucacuccu | 102 | acucacuccu | 107 |
| | CD33-CAG-5-U5 | cccccaccucccugggcccc | 103 | ccugggcccc | 108 |
| sgRNA | CD33-CAG-6 | guuccacagaacccaacaac | 6 | — | |
| hsgRNA | CD33-CAG-6-U1 | ucaucucuacccccaacuga | 109 | ccccaacuga | 112 |
| | CD33-CAG-6-U2 | ugguuucuggcaggaguaag | 110 | caggaguaag | 113 |
| | CD33-CAG-6-U3 | ugugguuucuggcaggagua | 111 | ggcaggagua | 114 |

TABLE 1B
G-to-A editing to create stop codon at TGG in CD33

| | | | SEQ | | SEQ |
|---|---|---|---|---|---|
| | | | ID | | ID |
| Type | Name | 20 nt | NO: | 10 nt | NO: |
| sgRNA | CD33-TGG-7 | cacucaccugcccacagcag | 7 | — | |
| hsgRNA | CD33-TGG-7-U1 | uggggaaacgagggucagcu | 115 | agggucagcu | 120 |
| | CD33-TGG-7-U2 | ggaccccuguggggaaacga | 116 | ggggaaacga | 121 |
| | CD33-TGG-7-U3 | ccauagccaggaccccugug | 117 | gaccccugug | 122 |
| | CD33-TGG-7-U4 | auccauagccaggaccccug | 118 | aggaccccug | 123 |
| | CD33-TGG-7-U5 | agaaauuuggauccauagcc | 119 | auccauagcc | 124 |
| sgRNA | CD33-TGG-8 | gaaccaguaaccaugaacug | 8 | — | |
| hsgRNA | CD33-TGG-8-U1 | uguggccacuggagaguccc | 125 | ggagaguccc | 128 |
| | CD33-TGG-8-U2 | ucuagcuuguuuguggccac | 126 | uuguggccac | 129 |
| | CD33-TGG-8-U3 | uucuugaucuagcuuguuug | 127 | agcuuguuug | 130 |
| sgRNA | CD33-TGG-9 | cggaaccaguaaccaugaac | 9 | — | |
| hsgRNA | CD33-TGG-9-U1 | uguggccacuggagaguccc | 131 | ggagaguccc | 134 |
| | CD33-TGG-9-U2 | ucuagcuuguuuguggccac | 132 | uuguggccac | 135 |
| | CD33-TGG-9-U3 | uucuugaucuagcuuguuug | 133 | agcuuguuug | 136 |
| sgRNA | CD33-TGG-10 | acaggcccaggacacagagc | 10 | — | |
| hsgRNA | CD33-TGG-10- | gacaaccaggagaagaucgg | 137 | agaagaucgg | 146 |
| | U1 | | | | |
| | CD33-TGG-10- | gcugacaaccaggagaagau | 138 | aggagaagau | 147 |
| | U2 | | | | |
| | CD33-TGG-10- | Ggugggggcagcugacaacc | 139 | gcugacaacc | 148 |
| | U3 | | | | |
| | CD33-TGG-10- | ccuggggcccagggaggugg | 140 | agggaggugg | 149 |
| | U4 | | | | |
| | CD33-TGG-10- | aguccuggggcccagggagg | 141 | cccagggagg | 150 |
| | U5 | | | | |
| | CD33-TGG-10- | aguaguccuggggcccaggg | 142 | gggcccaggg | 151 |
| | U6 | | | | |
| | CD33-TGG-10- | agugaguaguccuggggccc | 143 | ccuggggccc | 152 |
| | U7 | | | | |
| | CD33-TGG-10- | ccgaggagugaguaguccug | 144 | aguaguccug | 153 |
| | U8 | | | | |
| | CD33-TGG-10- | caccgaggagugaguagucc | 145 | ugaguagucc | 154 |
| | U9 | | | | |
| sgRNA | CD33-TGG-11 | gacaaccaggagaagaucgg | 11 | — | |
| hsgRNA | CD33-TGG-11- | aguaguccuggggcccaggg | 155 | gggcccaggg | 162 |
| | U1 | | | | |
| | CD33-TGG-11- | agugaguaguccuggggccc | 156 | ccuggggccc | 163 |
| | U2 | | | | |
| | CD33-TGG-11- | ccgaggagugaguaguccug | 157 | aguaguccug | 164 |
| | U3 | | | | |
| | CD33-TGG-11- | caccgaggagugaguagucc | 158 | ugaguagucc | 165 |
| | U4 | | | | |
| | CD33-TGG-11- | uggggugauuaugagcaccg | 159 | augagcaccg | 166 |
| | U5 | | | | |
| | CD33-TGG-11- | gccgugguccuggggccgug | 160 | uggggccgug | 167 |
| | U6 | | | | |
| | CD33-TGG-11- | agguuggugccgugguccug | 161 | cgugguccug | 168 |
| | U7 | | | | |

TABLE 1C
G-to-A editing to disrupt GU-AG splicing site in CD33

| | | | SEQ | | SEQ |
|---|---|---|---|---|---|
| | | | ID | | ID |
| Type | Name | 20 nt | NO: | 10 nt | NO: |
| sgRNA | CD33-GU-12 | ccacucaccugcccacagca | 12 | — | |
| hsgRNA | CD33-GU-12-U1 | uggggaaacgagggucagcu | 169 | agggucagcu | 174 |
| | CD33-GU-12-U2 | ggaccccuguggggaaacga | 170 | ggggaaacga | 175 |
| | CD33-GU-12-U3 | ccauagccaggaccccugug | 171 | gaccccugug | 176 |
| | CD33-GU-12-U4 | auccauagccaggaccccug | 172 | aggaccccug | 177 |
| | CD33-GU-12-U5 | agaaauuuggauccauagcc | 173 | auccauagcc | 178 |
| sgRNA | CD33-AG-13 | caagucuagugaggagaaag | 13 | — | |
| hsgRNA | CD33-AG-13-U1 | guucuagagugccagggaug | 179 | gccagggaug | 187 |
| | CD33-AG-13-U2 | ggccggguucuagagugcca | 180 | uagagugcca | 188 |
| | CD33-AG-13-U3 | uggccggguucuagagugcc | 181 | cuagagugcc | 189 |
| | CD33-AG-13-U4 | gucagguuuuuggaguggcc | 182 | uggaguggcc | 190 |
| | CD33-AG-13-U5 | agcaggucagguuuuuggag | 183 | guuuuuggag | 191 |
| | CD33-AG-13-U6 | cacagagcaggucagguuuu | 184 | gucagguuuu | 192 |
| | CD33-AG-13-U7 | cccaggacacagagcagguc | 185 | agagcagguc | 193 |
| | CD33-AG-13-U8 | acaggcccaggacacagagc | 186 | gacacagagc | 194 |
| sgRNA | CD33-AG-14 | acuuacaggugacguugagc | 14 | — | |
| hsgRNA | CD33-AG-14-U1 | cugucuccccuacacccuca | 195 | uacacccuca | 197 |
| | CD33-AG-14-U2 | ccugucuccccuacacccuc | 196 | cuacacccuc | 198 |
| sgRNA | CD33-AG-15 | aacaucuaggagaggaagag | 15 | — | |
| hsgRNA | CD33-AG-15-U1 | uuccuaccugagccaucucc | 199 | agccaucucc | 204 |
| | CD33-AG-15-U2 | aguaacagccccaggcgggg | 200 | ccaggcgggg | 205 |
| | CD33-AG-15-U3 | gucaguaacagccccaggcg | 201 | gccccaggcg | 206 |
| | CD33-AG-15-U4 | augucaguaacagccccagg | 202 | cagccccagg | 207 |
| | CD33-AG-15-U5 | ucaaugucaguaacagcccc | 203 | uaacagcccc | 208 |
| sgRNA | CD33-AG-16 | uuccuaccugagecaucucc | 16 | — | |
| hsgRNA | CD33-AG-16-U1 | ucaaugucaguaacagcccc | 209 | uaacagcccc | 210 |
| sgRNA | CD33-AG-17 | augcucacaugaagaagaug | 17 | — | |
| hsgRNA | CD33-AG-17-U1 | agguccauccucuucaccuc | 211 | ucuucaccuc | 214 |
| | CD33-AG-17-U2 | cuccaggacccuucuacacc | 212 | cuucuacacc | 215 |
| | CD33-AG-17-U3 | ccagcccucacagccccucc | 213 | cagccccucc | 216 |
| sgRNA | CD33-AG-18 | cacucugaugggagacacca | 18 | — | |
| hsgRNA | CD33-AG-18-U1 | auuccugcccacugcugucc | 217 | acugcugucc | 221 |
| | CD33-AG-18-U2 | ggcugacccugugguagggu | 218 | gugguagggu | 222 |
| | CD33-AG-18-U3 | gggaggcugacccuguggua | 219 | cccuguggua | 223 |
| | CD33-AG-18-U4 | caccggggaggcugacccug | 220 | gcugacccug | 224 |

TABLE 1D
C-to-T editing to create stop codon at CAA, CAG, or CGA in CD123

| | | | SEQ | | SEQ |
|---|---|---|---|---|---|
| | | | ID | | ID |
| Type | Name | 20 nt | NO: | 10 nt | NO: |
| sgRNA | CD123-CAA-1 | ugucuccugcaaacgaagga | 19 | — | |
| hsgRNA | CD123-CAA-1- | cguucccgaugguccuccuu | 225 | gguccuccuu | 228 |
| | U1 | | | | |
| | CD123-CAA-1- | uuccggagcugcguucccga | 226 | gcguucccga | 229 |
| | U2 | | | | |
| | CD123-CAA-1- | ggcaccucuguccugcguuc | 227 | uccugcguuc | 230 |
| | U3 | | | | |
| sgRNA | CD123-CAA-2 | uucucaaaguucccacaucc | 20 | — | |
| hsgRNA | CD123-CAA-2- | augcucagggaacacguauc | 231 | aacacguauc | 234 |
| | U1 | | | | |
| | CD123-CAA-2- | cacuacaaaacggaugcuca | 232 | cggaugcuca | 235 |
| | U2 | | | | |
| | CD123-CAA-2- | cgagugucuucacuacaaaa | 233 | cacuacaaaa | 236 |
| | U3 | | | | |
| sgRNA | CD123-CAG-3 | augcucagggaacacguauc | 21 | — | |
| hsgRNA | CD123-CAG-3- | accuuaccgcuuaccgcagc | 237 | uuaccgcagc | 239 |
| | U1 | | | | |
| | CD123-CAG-3- | gugcgggugccaucggcgug | 238 | caucggcgug | 240 |
| | U2 | | | | |
| sgRNA | CD123-CAG-4 | gaugcucagggaacacguau | 22 | — | |
| hsgRNA | CD123-CAG-4- | accuuaccgcuuaccgcagc | 241 | uuaccgcagc | 243 |
| | U1 | | | | |
| | CD123-CAG-4- | gugcgggugccaucggcgug | 242 | caucggcgug | 244 |
| | U2 | | | | |
| sgRNA | CD123-CAG-5 | ucacagauuggugaguagcc | 23 | — | |
| hsgRNA | CD123-CAG-5- | cggggcaggagcgcagccuu | 245 | gcgcagccuu | 250 |
| | U1 | | | | |
| | CD123-CAG-5- | cccacauccuggugcggggc | 246 | ggugcggggc | 251 |
| | U2 | | | | |
| | CD123-CAG-5- | aguucccacauccuggugcg | 247 | uccuggugcg | 252 |
| | U3 | | | | |
| | CD123-CAG-5- | aaaguucccacauccuggug | 248 | cauccuggug | 253 |
| | U4 | | | | |
| | CD123-CAG-5- | uucucaaaguucccacaucc | 249 | ucccacaucc | 254 |
| | U5 | | | | |
| sgRNA | CD123-CGA-6 | gacaucucucgacucuccag | 24 | — | |
| hsgRNA | CD123-CGA-6- | gaugcucagggaacacguau | 255 | gaacacguau | 258 |
| | U1 | | | | |
| | CD123-CGA-6- | cacuacaaaacggaugcuca | 256 | cggaugcuca | 259 |
| | U2 | | | | |
| | CD123-CGA-6- | cgagugucuucacuacaaaa | 257 | cacuacaaaa | 260 |
| | U3 | | | | |

TABLE 1E
G-to-A editing to create stop codon at TGG in CD123

| | | | SEQ | | SEQ |
|---|---|---|---|---|---|
| | | | ID | | ID |
| Type | Name | 20 nt | NO: | 10 nt | NO: |
| sgRNA | CD123-TGG-7 | ugagccaaaggaggaccauc | 25 | — | |
| hsgRNA | CD123-TGG-7-U1 | cguuugcaggagacagggca | 261 | agacagggca | 266 |
| | CD123-TGG-7-U2 | ucguuugcaggagacagggc | 262 | gagacagggc | 267 |
| | CD123-TGG-7-U3 | uccuucguuugcaggagaca | 263 | gcaggagaca | 268 |
| | CD123-TGG-7-U4 | uuccuucguuugcaggagac | 264 | ugcaggagac | 269 |
| | CD123-TGG-7-U5 | ucuuaccuuccuucguuugc | 265 | cuucguuugc | 270 |
| sgRNA | CD123-TGG-8 | ccugcccaaggcuucccacc | 26 | — | |
| hsgRNA | CD123-TGG-8-U1 | auccacgucaugaauccagc | 271 | ugaauccagc | 273 |
| | CD123-TGG-8-U2 | ggacguccgcgggggccccc | 272 | gggggccccc | 274 |

TABLE 1F
G-to-A editing to disrupt GU-AG splicing site in CD123

| | | | SEQ | | SEQ |
|---|---|---|---|---|---|
| | | | ID | | ID |
| Type | Name | 20 nt | NO: | 10 nt | NO: |
| sgRNA | CD123-GU-9 | ucuuaccuuccuucguuugc | 27 | — | |
| hsgRNA | CD123-GU-9-U1 | uacccccaccgcuccccagg | 275 | gcuccccagg | 277 |
| | CD123-GU-9-U2 | gucuacccccaccgcucccc | 276 | accgcucccc | 278 |
| sgRNA | CD123-AG-10 | uuuggaucuaaaacggugac | 28 | — | |
| hsgRNA | CD123-AG-10- | gagccuuugcuuucauccuu | 279 | uuucauccuu | 282 |
| | U1 | | | | |
| | CD123-AG-10- | cacauuucuguuaagguccc | 280 | uuaagguccc | 283 |
| | U2 | | | | |
| | CD123-AG-10- | uaucggucacauuucuguua | 281 | auuuuuu | 284 |
| | U3 | | | | |
| sgRNA | CD123-GU-11 | cucaccuguucugugauuac | 29 | — | |
| hsgRNA | CD123-GU-11- | cuuugcaaugucaaguacag | 285 | ucaaguacag | 286 |
| | U1 | | | | |
| sgRNA | CD123-AG-12 | ggucgcacucuagggguaaa | 30 | — | |
| hsgRNA | CD123-AG-12- | gaucagcagcgacguccgcc | 287 | gacguccgcc | 289 |
| | U1 | | | | |
| | CD123-AG-12- | gaucacgaagacacagacca | 288 | acacagacca | 290 |
| | U2 | | | | |
| sgRNA | CD123-AG-13 | uaccucggaggaaagagaaa | 31 | — | |
| hsgRNA | CD123-AG-13- | cgauggggucuuucauguga | 291 | uuucauguga | 296 |
| | U1 | | | | |
| | CD123-AG-13- | ccgauggggucuuucaugug | 292 | cuuucaugug | 297 |
| | U2 | | | | |
| | CD123-AG-13- | uuuggaagcugucaccgaug | 293 | gucaccgaug | 298 |
| | U3 | | | | |
| | CD123-AG-13- | guuuuggaagcugucaccga | 294 | cugucaccga | 299 |
| | U4 | | | | |
| | CD123-AG-13- | aacauaccagcuugucguuu | 295 | | 300 |
| | U5 | | | | |

TABLE 1G
G-to-A editing to create stop codon at TGG in CD47

| | | | SEQ | | SEQ |
|---|---|---|---|---|---|
| | | | ID | | ID |
| Type | Name | 20 nt | NO: | 10 nt | NO: |
| sgRNA | CD47-TGG-1 | uggagaaaaccaugaaacug | 32 | — | |
| hsgRNA | CD47-TGG-1-U1 | acaggaguauagcaaaaauu | 301 | agcaaaaauu | 304 |
| | CD47-TGG-1-U2 | aacaggaguauagcaaaaau | 302 | uagcaaaaau | 305 |
| | CD47-TGG-1-U3 | uaccaaacuguccccagaac | 303 | uccccagaac | 306 |

TABLE 1H
G-to-A editing to disrupt GU-AG splicing site in CD47

| | | | SEQ | | SEQ |
|---|---|---|---|---|---|
| | | | ID | | ID |
| Type | Name | 20 nt | NO: | 10 nt | NO: |
| sgRNA | CD47-GU-2 | cuuaccugggacgaaaagaa | 33 | — | |
| hsgRNA | CD47-GU-2-U1 | uaccuccugcguuccugccu | 307 | guuccugccu | 309 |
| | CD47-GU-2-U2 | uaaugcagcccuccucaccu | 308 | cuccucaccu | 310 |
| sgRNA | CD47-AG-3 | caaucgcuggaggaaggaaa | 34 | — | |
| hsgRNA | CD47-AG-3-U1 | aaccaauauggcaaugacga | 311 | gcaaugacga | 315 |
| | CD47-AG-3-U2 | uaucaccugaauaaccaaua | 312 | auaaccaaua | 316 |
| | CD47-AG-3-U3 | uccaaccacagcgaggauau | 313 | gcgaggauau | 317 |
| | CD47-AG-3-U4 | gacucaguccaaccacageg | 314 | aaccacagcg | 318 |

TABLE 11
C-to-T editing to create stop codon at CAA, CAG, or CGA in CD45

| | | | SEQ | | SEQ |
|---|---|---|---|---|---|
| | | | ID | | ID |
| Type | Name | 20 nt | NO: | 10 nt | NO: |
| sgRNA | CD45-CAA-1 | cacaucaaggaguaauuacc | 35 | — | |
| hsgRNA | CD45-CAA-1-U1 | cuuauucuuuuaacaggucc | 319 | uaacaggucc | 321 |
| | CD45-CAA-1-U2 | aacauucuuauucuuuuaac | 320 | uuuuuuaac | 322 |
| sgRNA | CD45-CAG-2 | gacucgcagacgcccucugc | 36 | — | |
| hsgRNA | CD45-CAG-2-U1 | ugaucucacuuuccuaccuu | 323 | uuccuaccuu | 324 |
| sgRNA | CD45-CAG-3 | ugcugcucagggaccacuga | 37 | — | |
| hsgRNA | CD45-CAG-3-U1 | uuuugucuaaaaagagcuac | 325 | aaagagcuac | 326 |
| sgRNA | CD45-CAG-4 | ugugcucaguacuggggaga | 38 | — | |
| hsgRNA | CD45-CAG-4-U1 | auccuuugcauauuucaaau | 327 | uauuucaaau | 328 |
| sgRNA | CD45-CAG-5 | ccccagaagaauuccucuga | 39 | — | |
| hsgRNA | CD45-CAG-5-U1 | gcagcuuccugcagaaccca | 329 | gcagaaccca | 331 |
| | CD45-CAG-5-U2 | ccaauauacaaacuggagug | 330 | aacuggagug | 332 |
| sgRNA | CD45-CGA-6 | cucgaugugaagaaggaaac | 40 | — | |
| hsgRNA | CD45-CGA-6-U1 | ugauuucuggaggaugauuu | 333 | aggaugauuu | 336 |
| | CD45-CGA-6-U2 | aaacuguugaugauuucugg | 334 | ugauuucugg | 337 |
| | CD45-CGA-6-U3 | uguuuaccuccuagguccca | 335 | cuagguccca | 338 |
| sgRNA | CD45- | aaggcgacagagaugccuga | 41 | — | |
| | CAG/CGA-7 | | | | |
| hsgRNA | CD45- | ccuggaagccgagaacaaag | 339 | gagaacaaag | 340 |
| | CAG/CGA-7-U1 | | | | |

TABLE 1J
G-to-A editing to create stop codon at TGG in CD45

| | | | SEQ | | SEQ |
|---|---|---|---|---|---|
| | | | ID | | ID |
| Type | Name | 20 nt | NO: | 10 nt | NO: |
| sgRNA | CD45-TGG-8 | gucauguuccagaccuggcu | 42 | — | |
| hsgRNA | CD45-TGG-8-U1 | gggccauuacggucccuggg | 341 | ggucccuggg | 342 |
| sgRNA | CD45-TGG-9 | ccacacuccaguuuguauau | 43 | — | |
| hsgRNA | CD45-TGG-9-U1 | aaucauagagauuaauuccu | 343 | | 344 |

TABLE 1K
G-to-A editing to disrupt GU-AG splicing site in CD45

| | | | SEQ | | SEQ |
|---|---|---|---|---|---|
| | | | ID | | ID |
| Type | Name | 20 nt | NO: | 10 nt | NO: |
| sgRNA | CD45-AG-10 | uccuguuaauuaauggaaaa | 44 | — | |
| hsgRNA | CD45-AG-10-U1 | gucacuugaaaguggaacac | 345 | aguggaacac | 350 |
| | CD45-AG-10-U2 | gguaaggggucacuugaaag | 346 | cacuugaaag | 351 |
| | CD45-AG-10-U3 | gcaguggugugaguagguaa | 347 | gaguagguaa | 352 |
| | CD45-AG-10-U4 | gagaaugcaguggugugagu | 348 | uggugugagu | 353 |
| | CD45-AG-10-U5 | gcuugcgggugagaaugcag | 349 | gagaaugcag | 354 |
| sgRNA | CD45-AG-11 | ugggacaucugcaaucagaa | 45 | — | |
| hsgRNA | CD45-AG-11-U1 | ugggucuguaggaaaggugc | 355 | ggaaaggugc | 364 |
| | CD45-AG-11-U2 | ggaaacugggucuguaggaa | 356 | ucuguaggaa | 365 |
| | CD45-AG-11-U3 | aauggggaaacugggucugu | 357 | cugggucugu | 366 |
| | CD45-AG-11-U4 | ugguugucaauggggaaacu | 358 | uggggaaacu | 367 |
| | CD45-AG-11-U5 | gcugagggugguugucaaug | 359 | guugucaaug | 368 |
| | CD45-AG-11-U6 | aggcugagggugguugucaa | 360 | ugguugucaa | 369 |
| | CD45-AG-11-U7 | guggugugcaaggcugaggg | 361 | aggcugaggg | 370 |
| | CD45-AG-11-U8 | agcuguggugugcaaggcug | 362 | ugcaaggcug | 371 |
| | CD45-AG-11-U9 | cagcagagcuguggugugca | 363 | guggugugca | 372 |
| sgRNA | CD45-GU-12 | gguaauaucaccuauuguug | 46 | — | |
| hsgRNA | CD45-GU-12-U1 | ccaggcacauggggauggug | 373 | ggggauggug | 378 |
| | CD45-GU-12-U2 | caucaccaggcacaugggga | 374 | cacaugggga | 379 |
| | CD45-GU-12-U3 | gagcacaucaccaggcacau | 375 | ccaggcacau | 380 |
| | CD45-GU-12-U4 | ccuugugagagcacaucacc | 376 | gcacaucacc | 381 |
| | CD45-GU-12-U5 | cccgagguagagugggugga | 377 | agugggugga | 382 |
| sgRNA | CD45-GU-13 | uuaccacauguuggcuuaga | 47 | — | |
| hsgRNA | CD45-GU-13-U1 | uaaagugcuauuccaaagug | 383 | uuccaaagug | 384 |

TABLE 1L
C-to-T editing to create stop codon at CAG in CLL1

| | | | SEQ | | SEQ |
|---|---|---|---|---|---|
| | | | ID | | ID |
| Type | Name | 20 nt | NO: | 10 nt | NO: |
| sgRNA | CLL1-CAG-1 | aacauggcaggagaguaaaa | 48 | — | |
| hsgRNA | CLL1-CAG-1-U1 | aaggagauggauuuggcaua | 385 | auuuggcaua | 389 |
| | CLL1-CAG-1-U2 | cuuguccaaggagauggauu | 386 | gagauggauu | 390 |
| | CLL1-CAG-1-U3 | guaagccuuguccaaggaga | 387 | uccaaggaga | 391 |
| | CLL1-CAG-1-U4 | acaaauguaagccuugucca | 388 | gccuugucca | 392 |

TABLE 1M
G-to-A editing to create stop codon at TGG in CLL1

| | | | SEQ | | SEQ |
|---|---|---|---|---|---|
| | | | ID | | ID |
| Type | Name | 20 nt | NO: | 10 nt | NO: |
| sgRNA | CLL1-TGG-2 | caaauccaucuccuuggaca | 49 | — | |
| hsgRNA | CLL1-TGG-2-U1 | auguuuggacaucaucacuu | 393 | aucaucacuu | 396 |
| | CLL1-TGG-2-U2 | uuuuacucuccugccauguu | 394 | cugccauguu | 397 |
| | CLL1-TGG-2-U3 | gcuggcauucugagcagcac | 395 | ugagcagcac | 398 |
| sgRNA | CLL1-TGG-3 | uuaugccaaauccaucuccu | 50 | — | |
| hsgRNA | CLL1-TGG-3-U1 | auguuuggacaucaucacuu | 399 | aucaucacuu | 402 |
| | CLL1-TGG-3-U2 | uuuuacucuccugccauguu | 400 | cugccauguu | 403 |
| | CLL1-TGG-3-U3 | gcuggcauucugagcagcac | 401 | ugagcagcac | 404 |

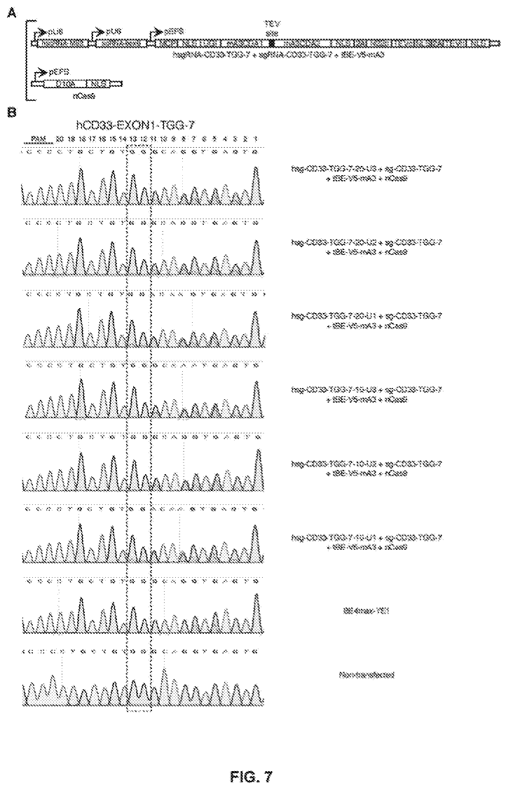

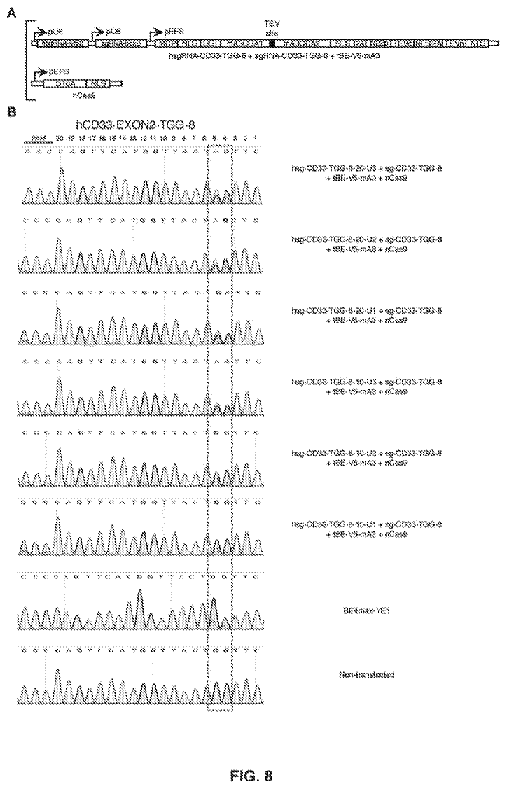

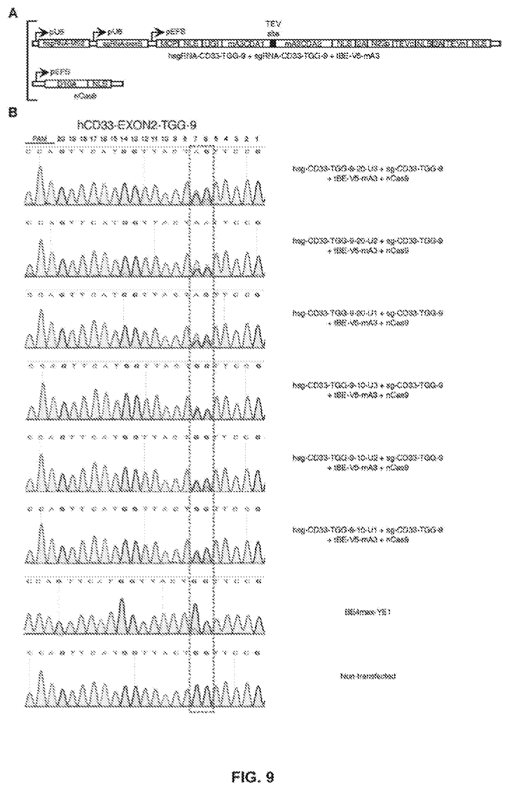

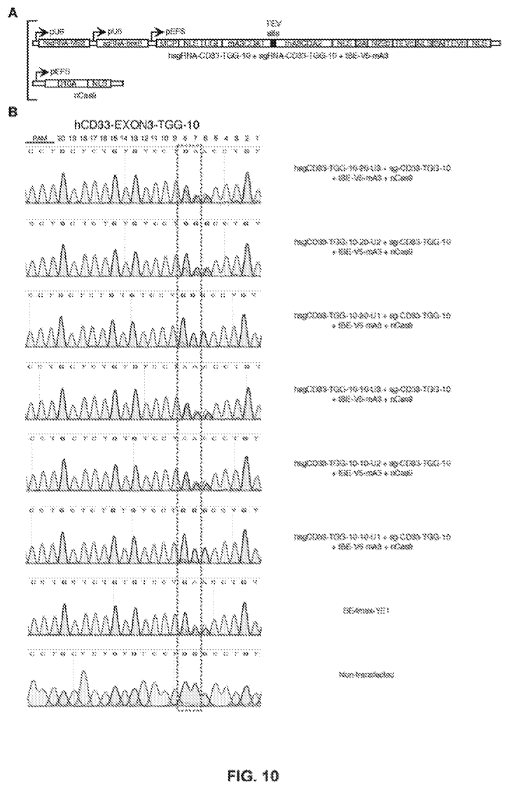

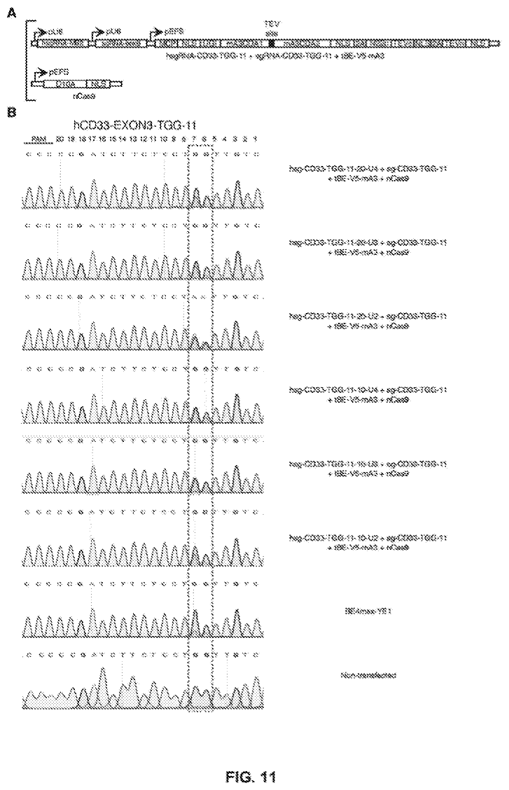

Next, this example used tBE to induce G-to-A (C-to-T on the opposite strand) base editing in the codon of TGG (Trp) in CD33 to create the TGA, TAG or TAA stop codon (Table 1B, FIG. 7-11). From the sanger sequencing results, it was confirmed that tBE induced higher base editing efficiencies than BE4max-YE1 at all these target sites, e.g., the target sites for hsgRNA-CD33-TGG-8-20/10-U3/2/1/sgRNA-CD33-TGG-8 (FIG. 8B).

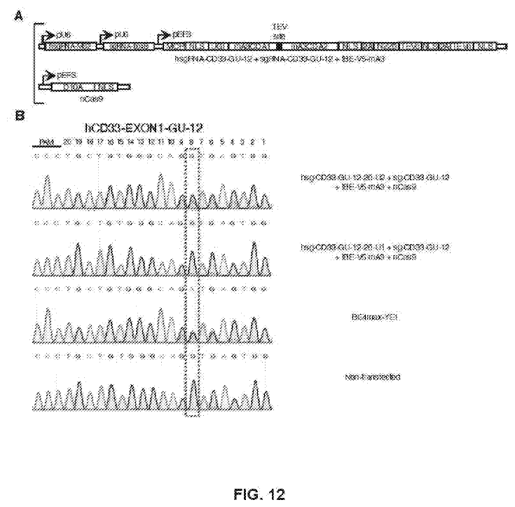

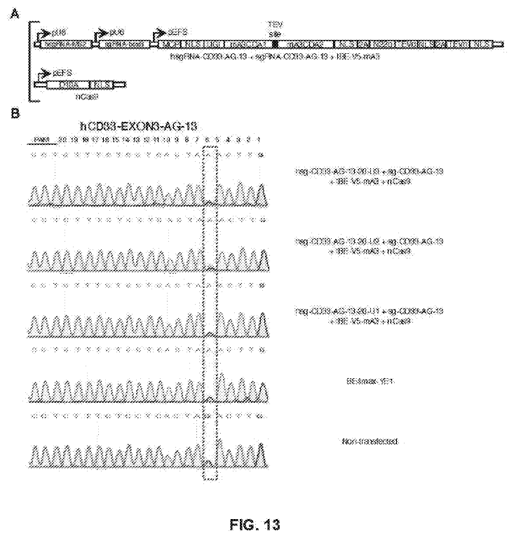

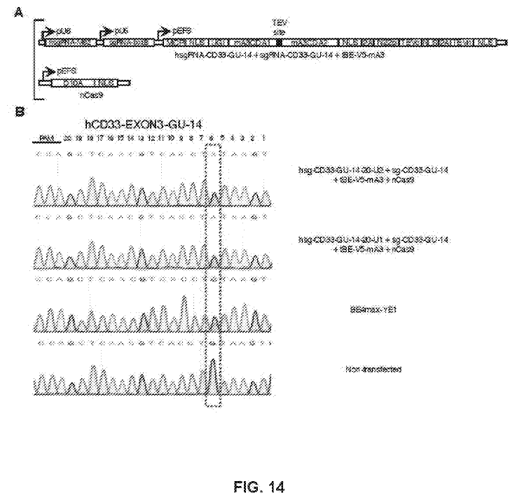

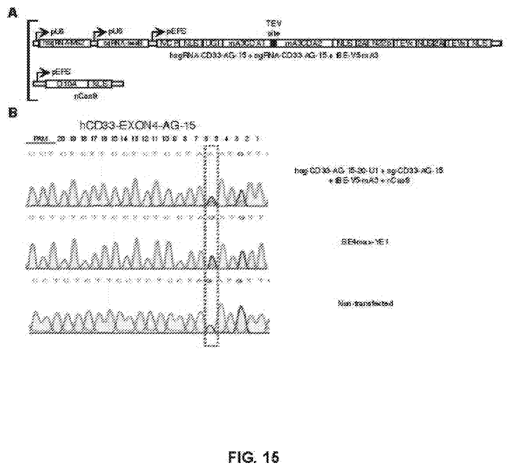

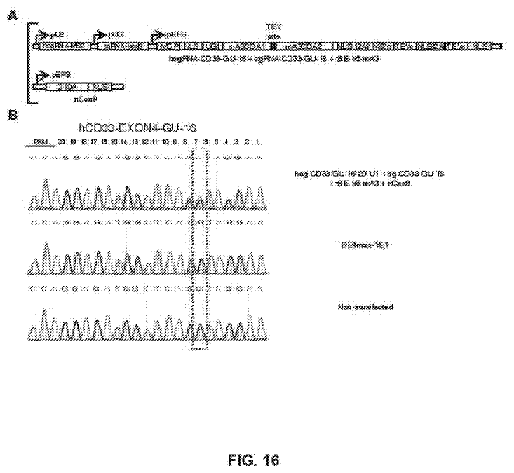

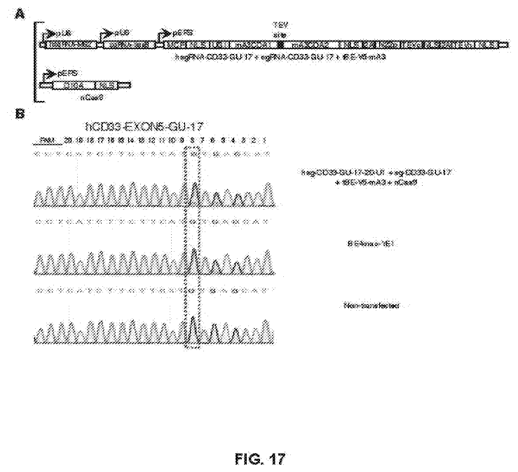

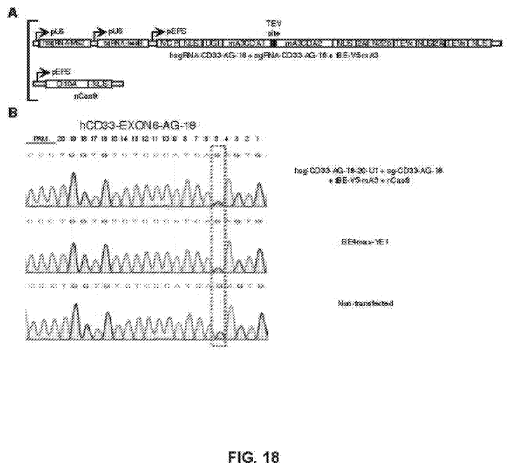

Then, this example used tBE to induce G-to-A base editing in 5′ GU or 3′ AG splice site to disrupt GU-AG canonical splicing rule (Table 1C, FIG. 12-18). From the sanger sequencing results, it was confirmed that tBE induced higher base editing efficiencies than BE4max-YE1 at most of these target sites, e.g., the target sites for hsgRNA-CD33-GU-14-20-U2/1/sgRNA-CD33-GU-14 (FIG. 14B).

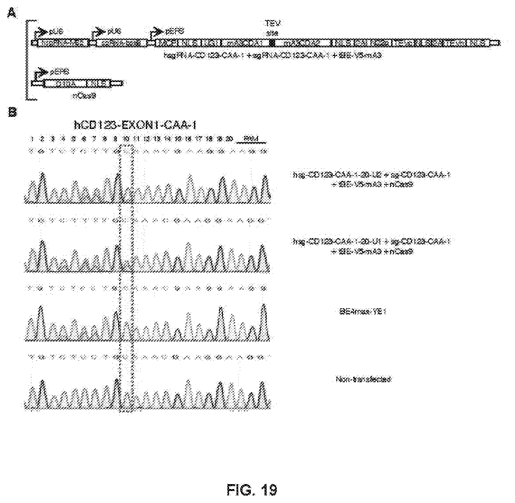

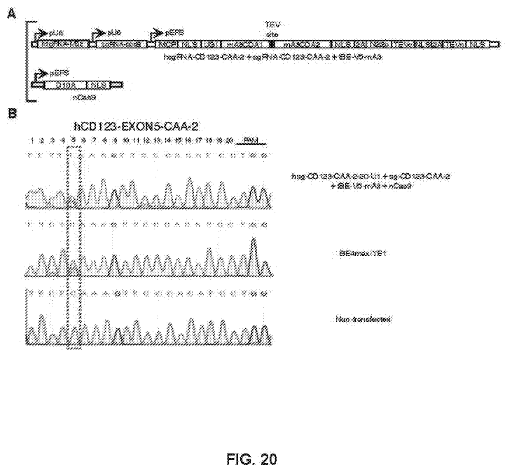

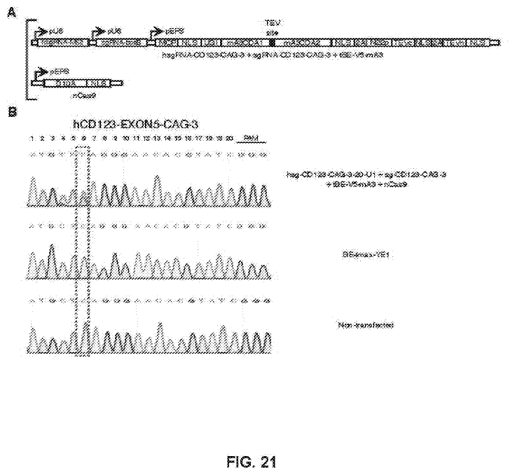

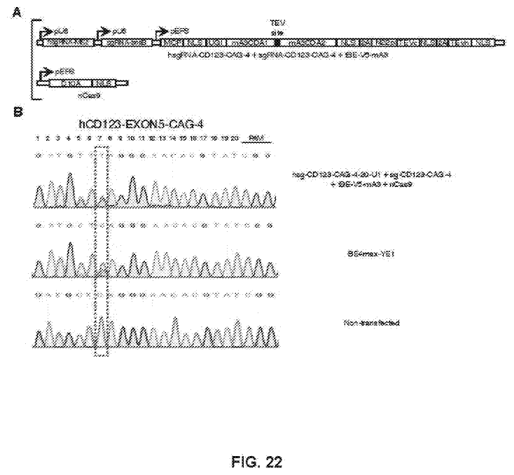

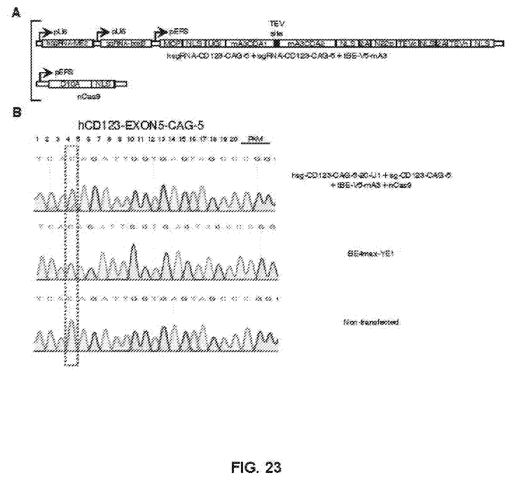

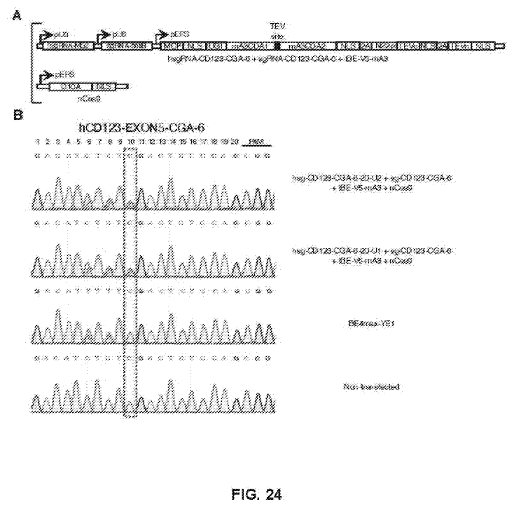

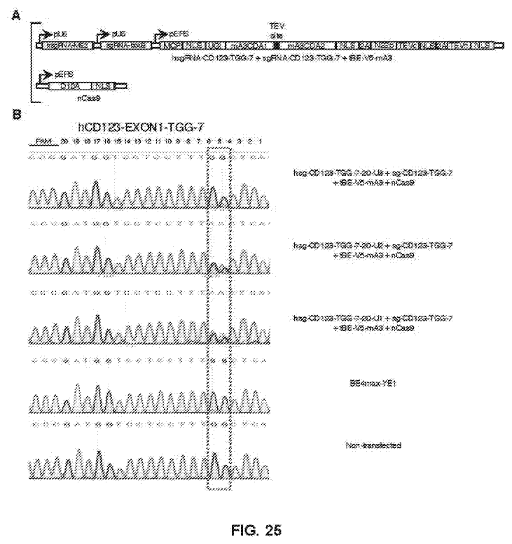

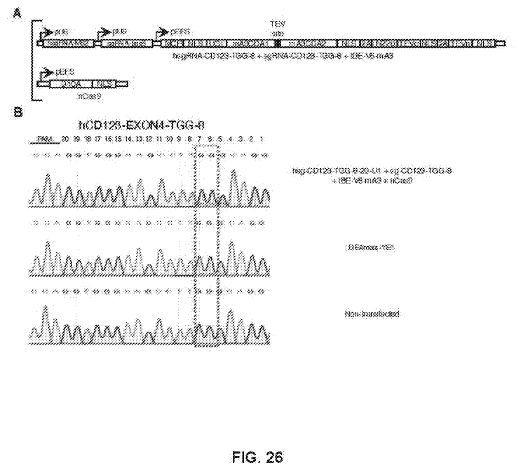

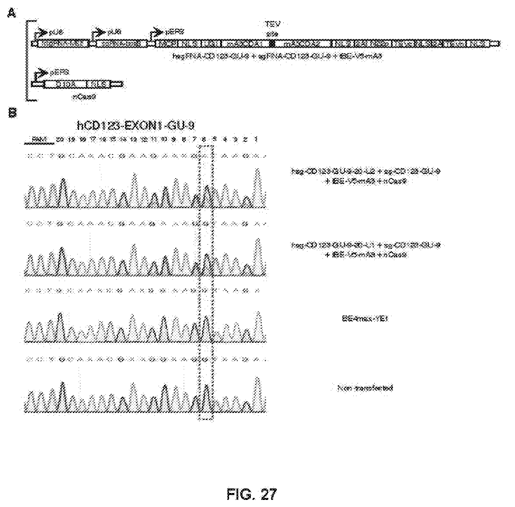

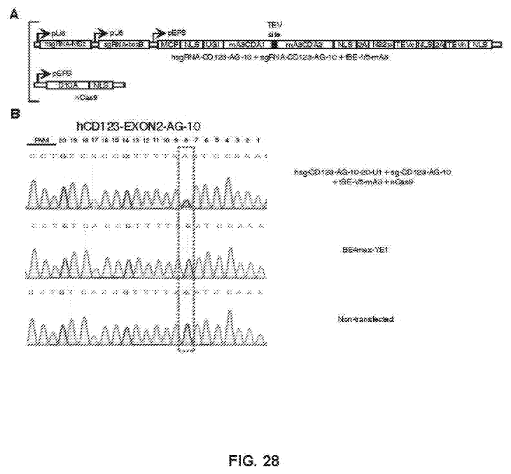

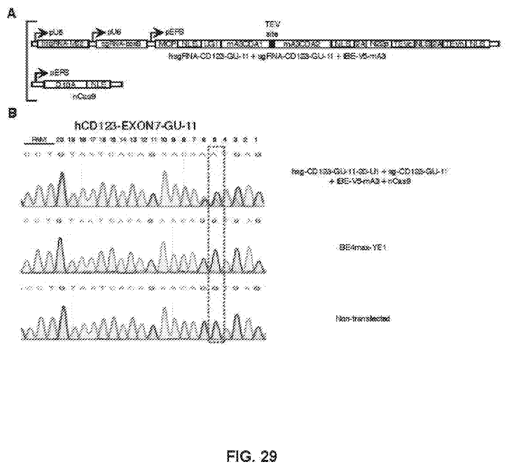

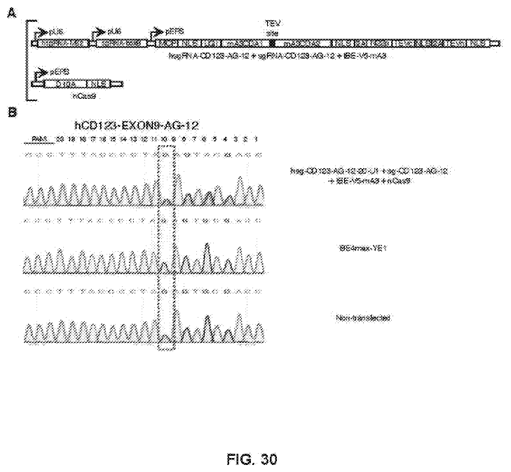

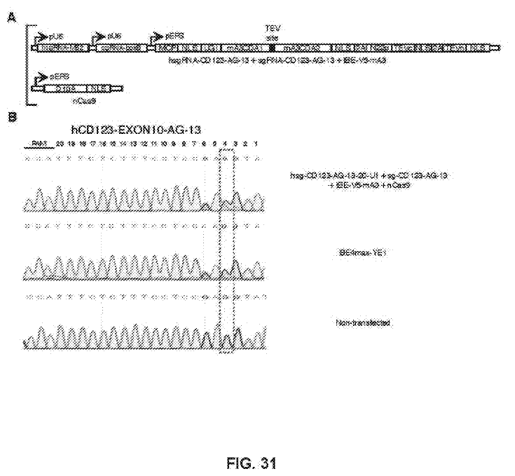

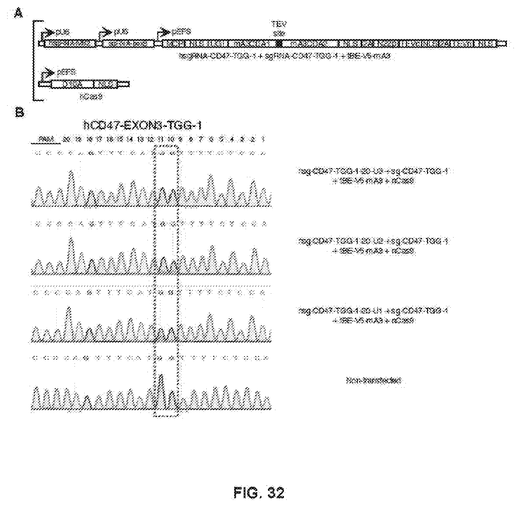

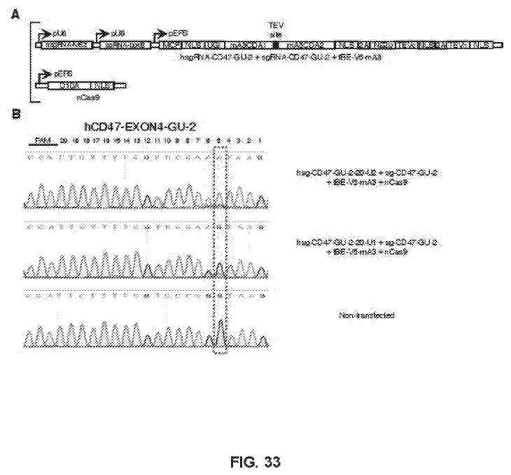

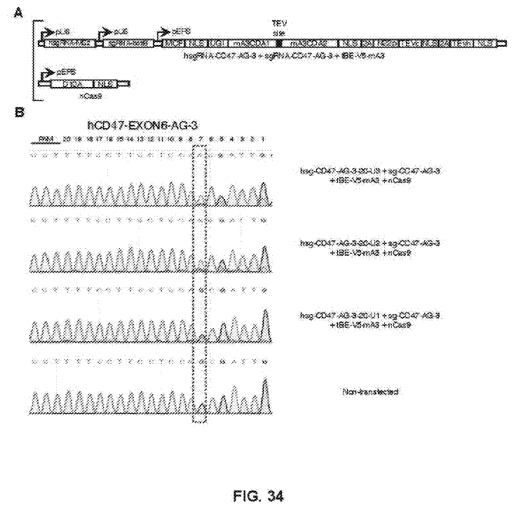

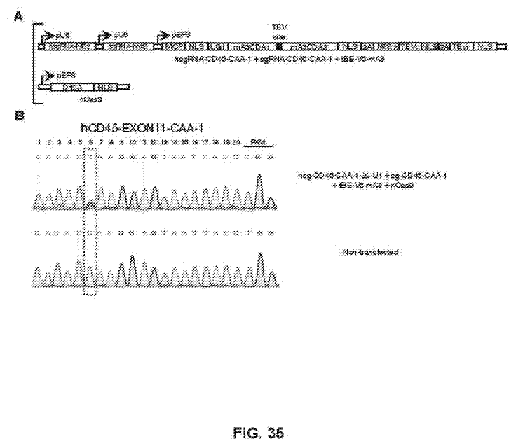

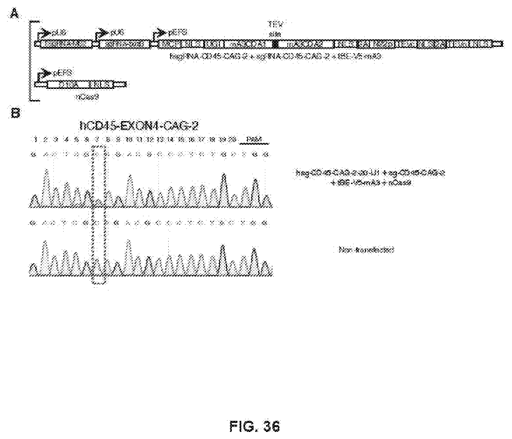

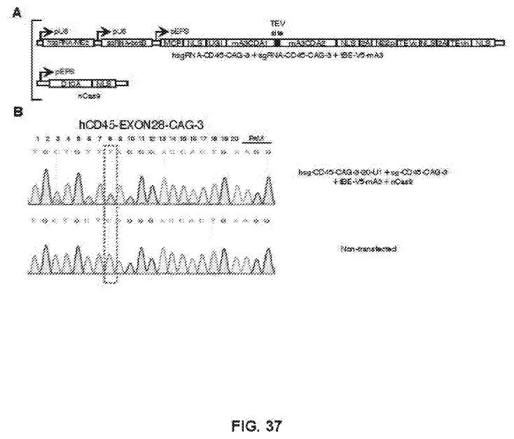

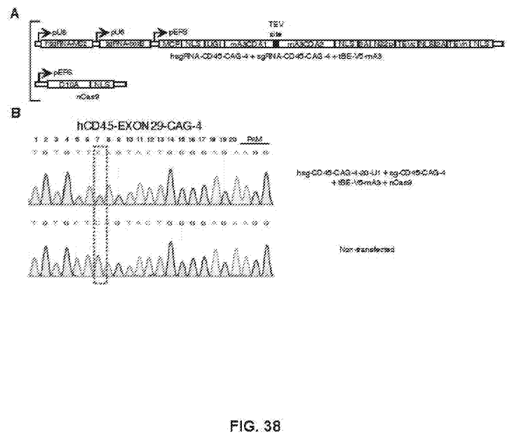

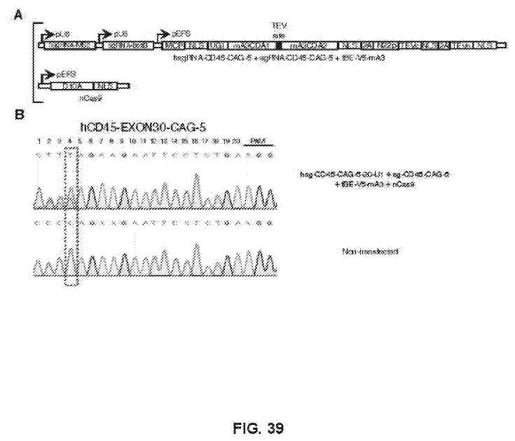

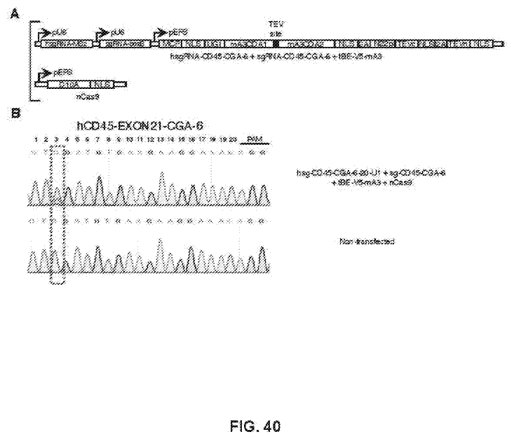

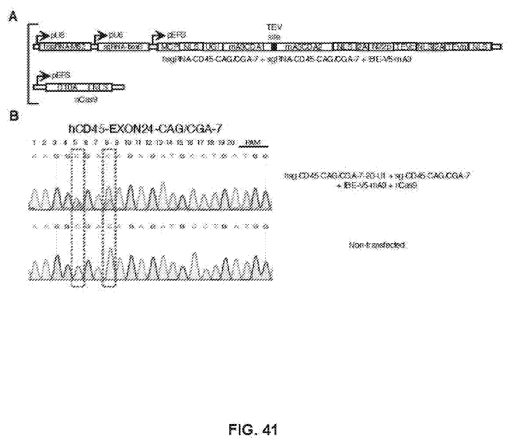

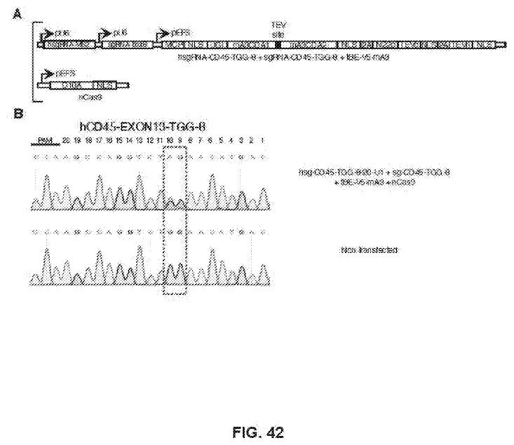

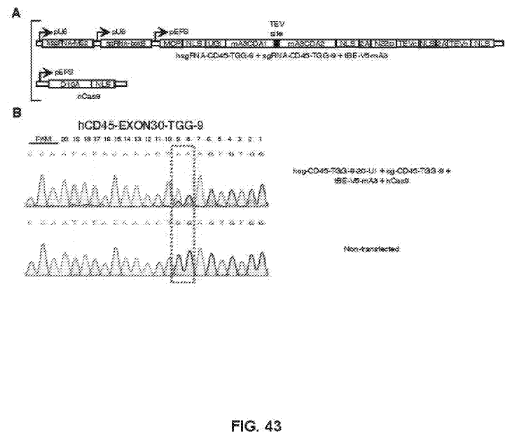

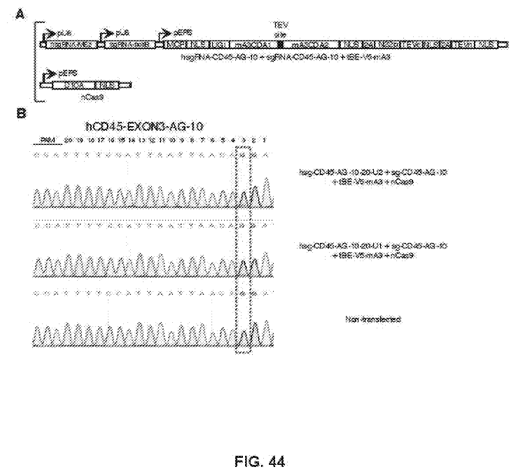

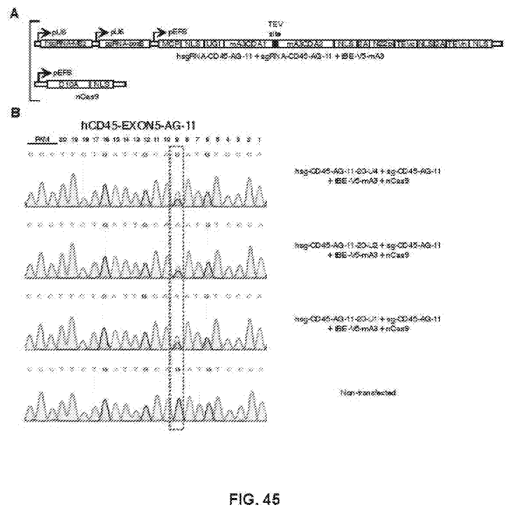

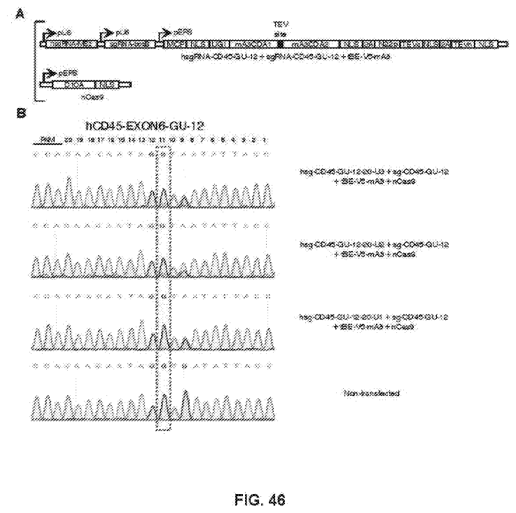

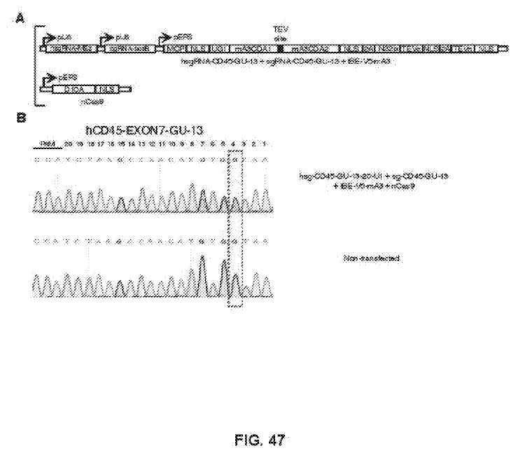

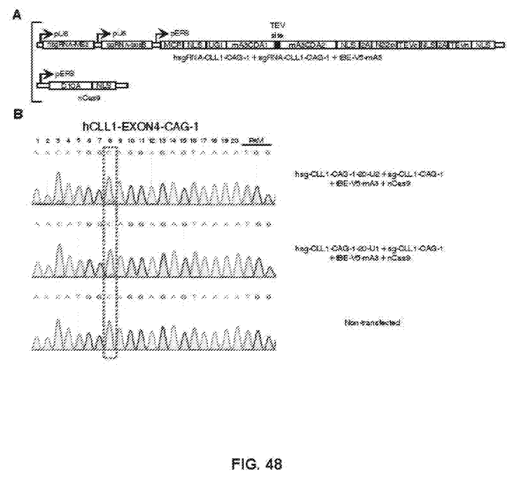

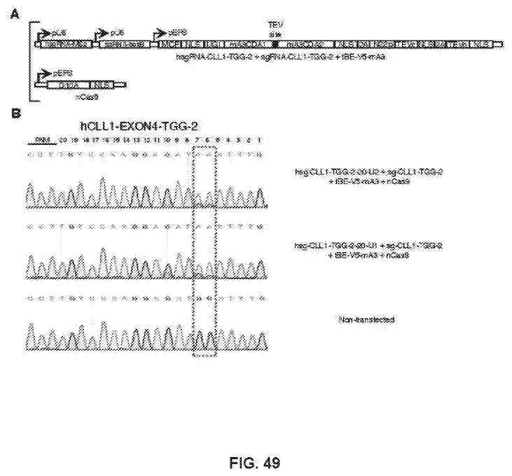

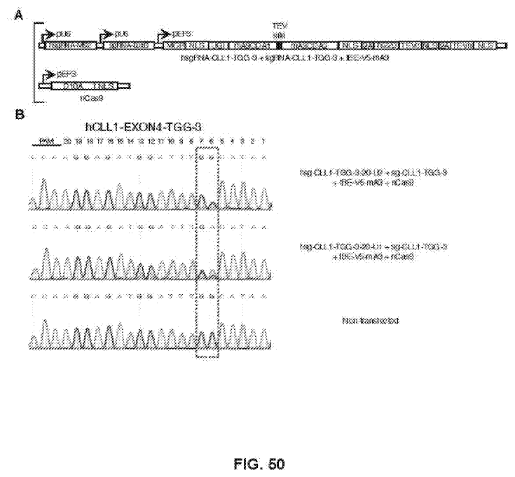

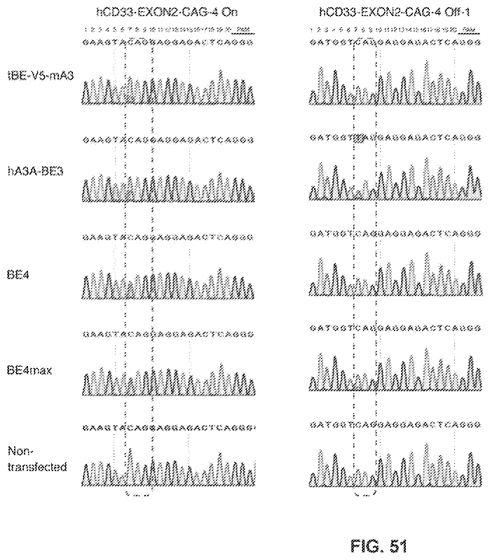

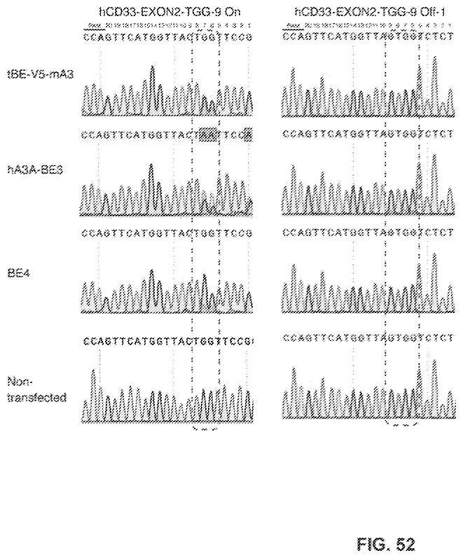

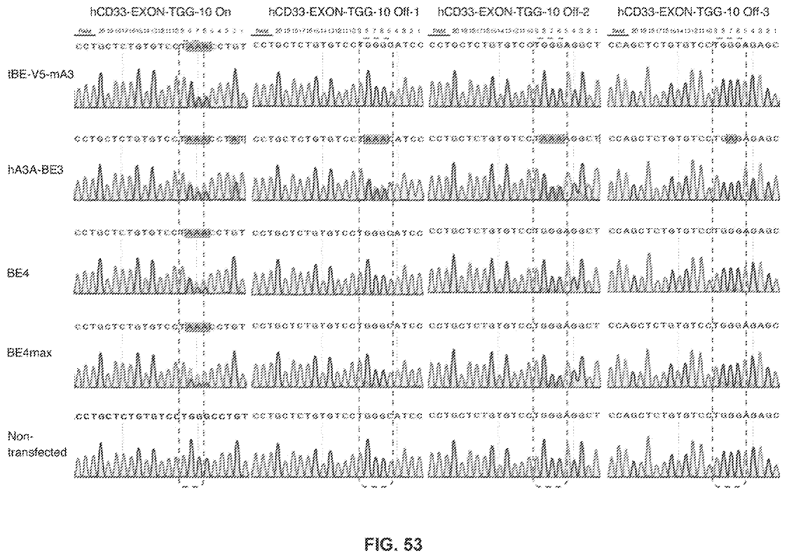

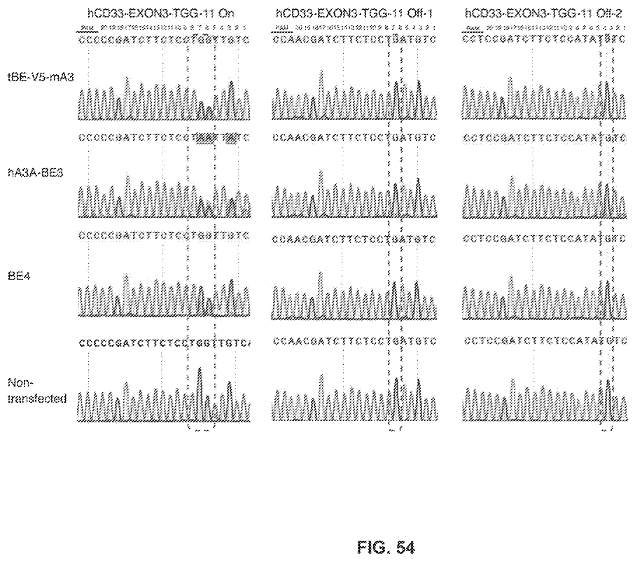

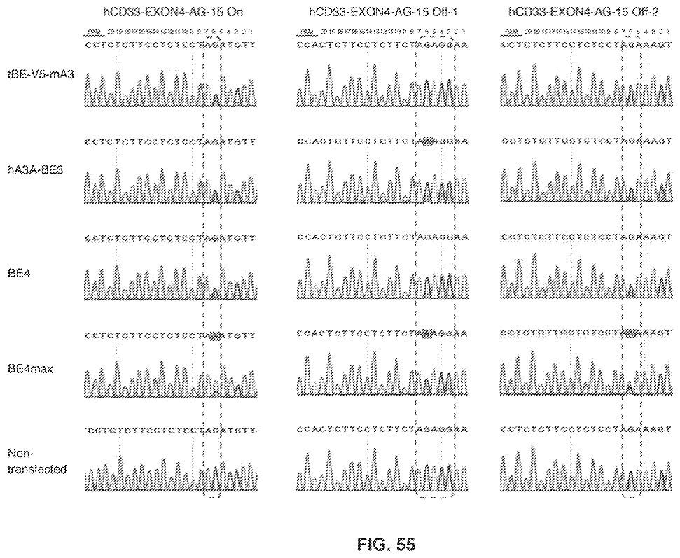

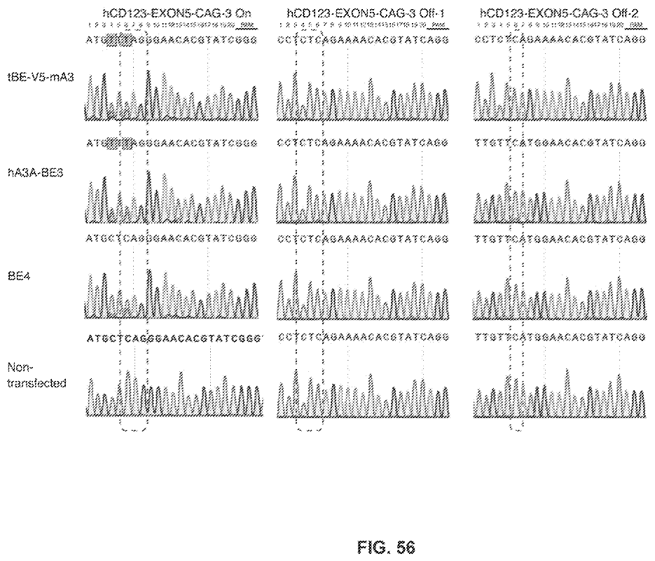

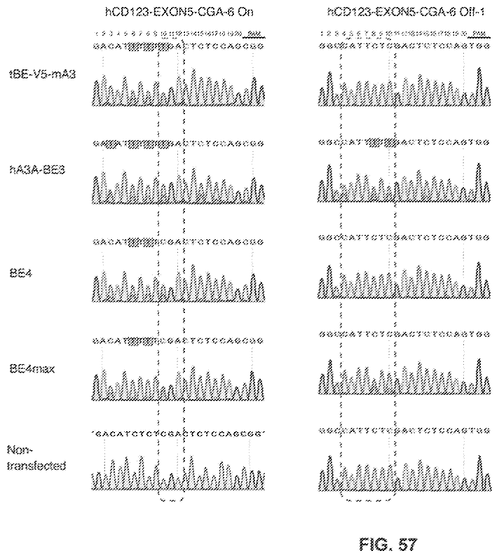

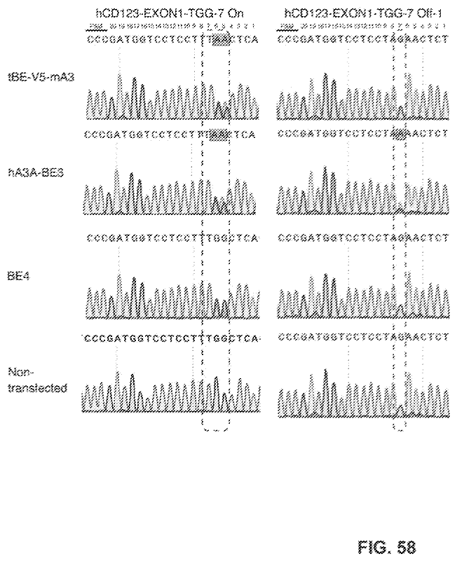

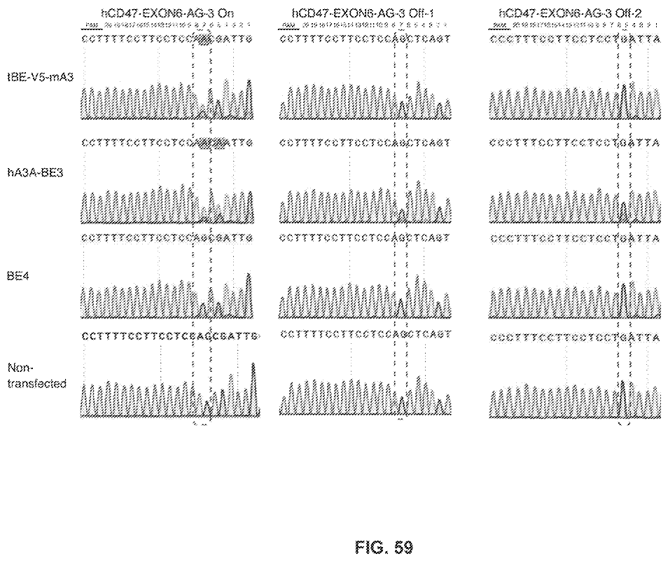

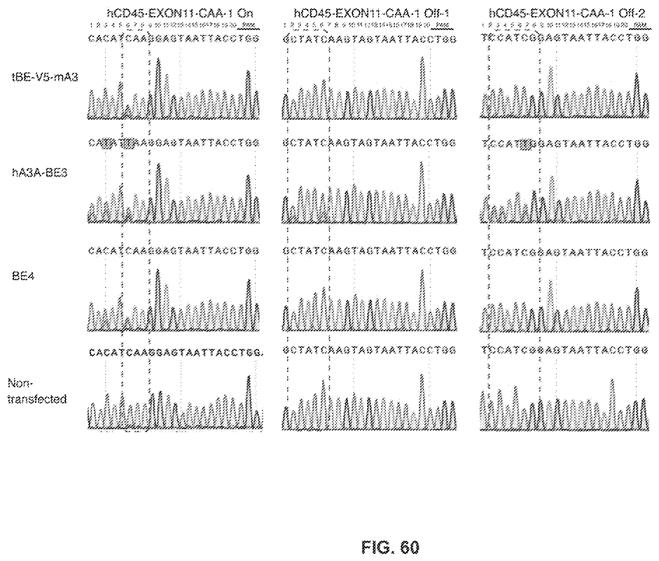

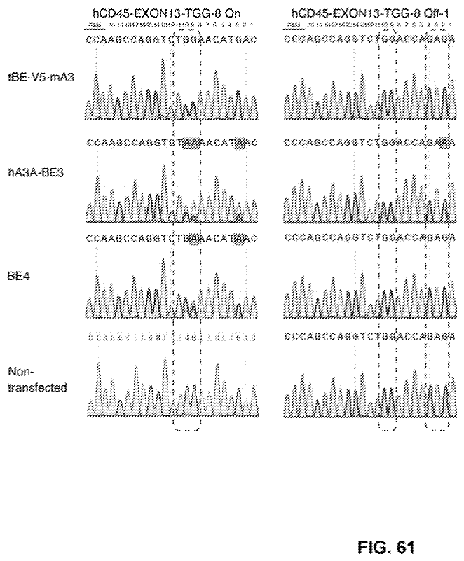

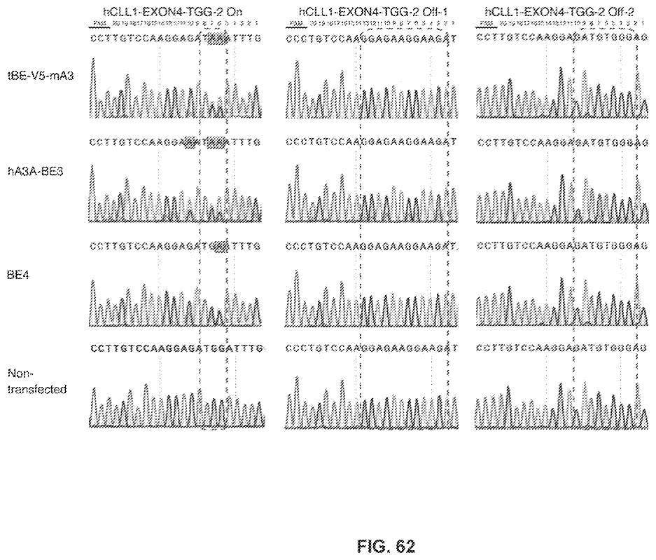

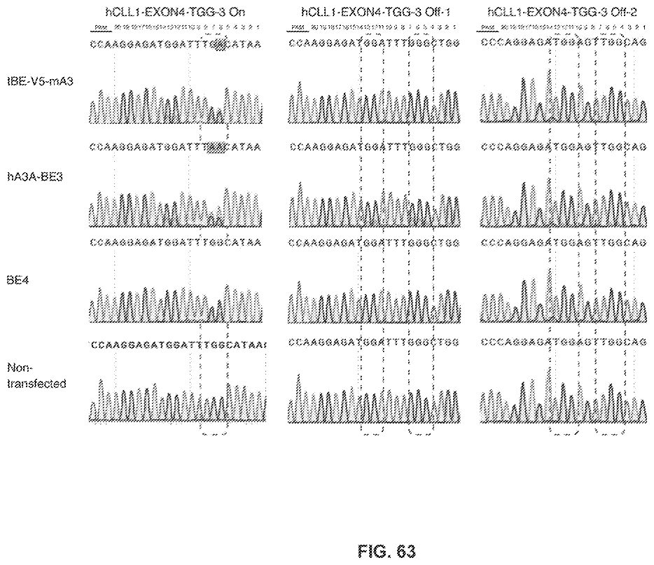

Similar experiments were conducted for the CD123, CD47, CD45 and CLL1 genes. The sgRNA/hsgRNA sequences are provided in Tables 1D-1M. As shown in FIG. 19-50, with these sgRNA/hsgRNA sequences, the tBE technology achieved excellent editing efficiencies. As shown in FIG. 51-63, these sgRNA/hsgRNA sequences induced greatly reduced or even no off-target editing.

The base editors and base editing method, along with the designed sgRNA/hsgRNA sequences, therefore, can perform high-specificity and high-efficiency base editing in the genome of various eukaryotes. Furthermore, the tBE system, which contains Cas9 nickase (D10A), is less toxic to cells than Cas9 nuclease as Cas9 nickase activates a lower level of p53-mediated DDR.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for reducing the biological activity of the CD33, CD123, CD47, CD45 or CLL1 gene in a cell to reduce toxicity in a patient undergoing a therapy targeting a cell surface antigen on a cancer cell, comprising administering to the patient, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 1 | SEQ ID NO: 57-62 |
| SEQ ID NO: 2 | SEQ ID NO: 69-74 |
| SEQ ID NO: 4 | SEQ ID NO: 93-98 |
| SEQ ID NO: 5 | SEQ ID NO: 104-108 |
| SEQ ID NO: 6 | SEQ ID NO: 112-114 |
| SEQ ID NO: 9 | SEQ ID NO: 134-136 |
| SEQ ID NO: 10 | SEQ ID NO: 146-154 |
| SEQ ID NO: 11 | SEQ ID NO: 162-168 |
| SEQ ID NO: 12 | SEQ ID NO: 174-178 |
| SEQ ID NO: 20 | SEQ ID NO: 234-236 |
| SEQ ID NO: 21 | SEQ ID NO: 239-240 |
| SEQ ID NO: 22 | SEQ ID NO: 243-244 |
| SEQ ID NO: 24 | SEQ ID NO: 258-260 |
| SEQ ID NO: 25 | SEQ ID NO: 266-270 |
| SEQ ID NO: 34 | SEQ ID NO: 315-318 |
| SEQ ID NO: 35 | SEQ ID NO: 321-322 |
| SEQ ID NO: 42 | SEQ ID NO: 342 |
| SEQ ID NO: 49 | SEQ ID NO: 396-398 |
| SEQ ID NO: 50 | SEQ ID NO: 402-404. |

2. A method for reducing the biological activity of the CD33 gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 1 | SEQ ID NO: 57-62 |
| SEQ ID NO: 2 | SEQ ID NO: 69-74 |
| SEQ ID NO: 3 | SEQ ID NO: 81-86 |
| SEQ ID NO: 4 | SEQ ID NO: 93-98 |
| SEQ ID NO: 5 | SEQ ID NO: 104-108 |
| SEQ ID NO: 6 | SEQ ID NO: 112-114 |
| SEQ ID NO: 7 | SEQ ID NO: 120-124 |
| SEQ ID NO: 8 | SEQ ID NO: 128-130 |
| SEQ ID NO: 9 | SEQ ID NO: 134-136 |
| SEQ ID NO: 10 | SEQ ID NO: 146-154 |
| SEQ ID NO: 11 | SEQ ID NO: 162-168 |
| SEQ ID NO: 12 | SEQ ID NO: 174-178 |
| SEQ ID NO: 13 | SEQ ID NO: 187-194 |
| SEQ ID NO: 14 | SEQ ID NO: 197-198 |
| SEQ ID NO: 15 | SEQ ID NO: 204-208 |
| SEQ ID NO: 16 | SEQ ID NO: 210 |
| SEQ ID NO: 17 | SEQ ID NO: 214-216 |
| SEQ ID NO: 18 | SEQ ID NO: 221-224. |

3. The method of claim 2, wherein the sgRNA and the hsgRNA, respectively, comprise the nucleic acid sequences selected from:

| sgRNA | hsgRNA |
|---|---|
| SEQ ID NO: 1 | SEQ ID NO: 51-56 |
| SEQ ID NO: 2 | SEQ ID NO: 63-68 |
| SEQ ID NO: 3 | SEQ ID NO: 75-80 |
| SEQ ID NO: 4 | SEQ ID NO: 87-92 |
| SEQ ID NO: 5 | SEQ ID NO: 99-103 |
| SEQ ID NO: 6 | SEQ ID NO: 109-111 |
| SEQ ID NO: 7 | SEQ ID NO: 115-119 |
| SEQ ID NO: 8 | SEQ ID NO: 125-127 |
| SEQ ID NO: 9 | SEQ ID NO: 131-133 |
| SEQ ID NO: 10 | SEQ ID NO: 137-145 |
| SEQ ID NO: 11 | SEQ ID NO: 155-161 |
| SEQ ID NO: 12 | SEQ ID NO: 169-173 |
| SEQ ID NO: 13 | SEQ ID NO: 179-186 |
| SEQ ID NO: 14 | SEQ ID NO: 195-196 |
| SEQ ID NO: 15 | SEQ ID NO: 199-203 |
| SEQ ID NO: 16 | SEQ ID NO: 209 |
| SEQ ID NO: 17 | SEQ ID NO: 211-213 |
| SEQ ID NO: 18 | SEQ ID NO: 217-220. |

4. A method for reducing the biological activity of the CD123 gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 19 | SEQ ID NO: 228-230 |
| SEQ ID NO: 20 | SEQ ID NO: 234-236 |
| SEQ ID NO: 21 | SEQ ID NO: 239-240 |
| SEQ ID NO: 22 | SEQ ID NO: 243-244 |
| SEQ ID NO: 23 | SEQ ID NO: 250-254 |
| SEQ ID NO: 24 | SEQ ID NO: 258-260 |
| SEQ ID NO: 25 | SEQ ID NO: 266-270 |
| SEQ ID NO: 26 | SEQ ID NO: 273-274 |
| SEQ ID NO: 27 | SEQ ID NO: 277-278 |
| SEQ ID NO: 28 | SEQ ID NO: 282-284 |
| SEQ ID NO: 29 | SEQ ID NO: 286 |
| SEQ ID NO: 30 | SEQ ID NO: 289-290 |
| SEQ ID NO: 31 | SEQ ID NO: 296-300. |

5. The method of claim 4, wherein the sgRNA and the hsgRNA, respectively, comprise the nucleic acid sequences selected from:

| sgRNA | hsgRNA |
|---|---|
| SEQ ID NO: 19 | SEQ ID NO: 225-227 |
| SEQ ID NO: 20 | SEQ ID NO: 231-233 |
| SEQ ID NO: 21 | SEQ ID NO: 237-238 |
| SEQ ID NO: 22 | SEQ ID NO: 237-238 |
| SEQ ID NO: 23 | SEQ ID NO: 245-249 |
| SEQ ID NO: 24 | SEQ ID NO: 255-257 |
| SEQ ID NO: 25 | SEQ ID NO: 261-265 |
| SEQ ID NO: 26 | SEQ ID NO: 271-272 |
| SEQ ID NO: 27 | SEQ ID NO: 275-276 |
| SEQ ID NO: 28 | SEQ ID NO: 279-281 |
| SEQ ID NO: 29 | SEQ ID NO: 285 |
| SEQ ID NO: 30 | SEQ ID NO: 287-288 |
| SEQ ID NO: 31 | SEQ ID NO: 291-295. |

6. A method for reducing the biological activity of the CD47 gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 32 | SEQ ID NO: 304-306 |
| SEQ ID NO: 33 | SEQ ID NO: 309-310 |
| SEQ ID NO: 34 | SEQ ID NO: 315-318. |

7. The method of claim 6, wherein the sgRNA and the hsgRNA, respectively, comprise the nucleic acid sequences selected from:

| sgRNA | hsgRNA |
|---|---|
| SEQ ID NO: 32 | SEQ ID NO: 301-303 |
| SEQ ID NO: 33 | SEQ ID NO: 307-308 |
| SEQ ID NO: 34 | SEQ ID NO: 311-314. |

8. A method for reducing the biological activity of the CD45 gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 35 | SEQ ID NO: 321-322 |
| SEQ ID NO: 36 | SEQ ID NO: 324 |
| SEQ ID NO: 37 | SEQ ID NO: 326 |
| SEQ ID NO: 38 | SEQ ID NO: 328 |
| SEQ ID NO: 39 | SEQ ID NO: 331-332 |
| SEQ ID NO: 40 | SEQ ID NO: 336-338 |
| SEQ ID NO: 41 | SEQ ID NO: 340 |
| SEQ ID NO: 42 | SEQ ID NO: 342 |
| SEQ ID NO: 43 | SEQ ID NO: 344 |
| SEQ ID NO: 44 | SEQ ID NO: 350-354 |
| SEQ ID NO: 45 | SEQ ID NO: 364-372 |
| SEQ ID NO: 46 | SEQ ID NO: 378-382 |
| SEQ ID NO: 47 | SEQ ID NO: 384. |

9. The method of claim 8, wherein the sgRNA and the hsgRNA, respectively, comprise the nucleic acid sequences selected from:

| sgRNA | hsgRNA |
|---|---|
| SEQ ID NO: 35 | SEQ ID NO: 319-320 |
| SEQ ID NO: 36 | SEQ ID NO: 323 |
| SEQ ID NO: 37 | SEQ ID NO: 325 |
| SEQ ID NO: 38 | SEQ ID NO: 327 |
| SEQ ID NO: 39 | SEQ ID NO: 329-330 |
| SEQ ID NO: 40 | SEQ ID NO: 333-335 |
| SEQ ID NO: 41 | SEQ ID NO: 339 |
| SEQ ID NO: 42 | SEQ ID NO: 341 |
| SEQ ID NO: 43 | SEQ ID NO: 343 |
| SEQ ID NO: 44 | SEQ ID NO: 345-349 |
| SEQ ID NO: 45 | SEQ ID NO: 355-363 |
| SEQ ID NO: 46 | SEQ ID NO: 373-377 |
| SEQ ID NO: 47 | SEQ ID NO: 383. |

10. A method for reducing the biological activity of the CLL1 gene in a cell, comprising introducing into the cell a CRISPR-associated (Cas) protein, a nucleobase deaminase, a single-guide RNA (sgRNA), and a helper single-guide RNA (hsgRNA), wherein the Cas protein, the nucleobase deaminase, the sgRNA, and the hsgRNA are preferably introduced into the cell by one or more encoding polynucleotides, and wherein the sgRNA and the hsgRNA, respectively, comprise the nucleic acid sequences selected from:

| sgRNA | hsgRNA |
|---|---|
| SEQ ID NO: 48 | SEQ ID NO: 389-392 |
| SEQ ID NO: 49 | SEQ ID NO: 396-398 |
| SEQ ID NO: 50 | SEQ ID NO: 402-404. |

11. The method of claim 10, wherein the sgRNA and the hsgRNA, respectively, comprise the nucleic acid sequences selected from:

| sgRNA | hsgRNA |
|---|---|
| SEQ ID NO: 48 | SEQ ID NO: 385-388 |
| SEQ ID NO: 49 | SEQ ID NO: 393-395 |
| SEQ ID NO: 50 | SEQ ID NO: 399-401. |

12. The method of any one of claims 1-11, wherein the nucleobase deaminase is a cytidine deaminase.

13. The method of claim 12, wherein the cytidine deaminase is selected from the group consisting of APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4) and AICDA (AID).

14. The method of any one of claims 1-13, further comprising introducing into the cell a nucleobase deaminase inhibitor, fused to the nucleobase deaminase, via a protease cleavage site.

15. The method of claim 14, wherein the nucleobase deaminase inhibitor is an inhibitory domain of a nucleobase deaminase.

16. The method of claim 14, wherein the nucleobase deaminase inhibitor is an inhibitory domain of a cytidine deaminase.

17. The method of any one of claims 1-16, further comprising introducing into the cell a protease that is capable of cleaving at the protease cleavage site.

18. The method of claim 17, wherein the protease is selected from the group consisting of TuMV protease, PPV protease, PVY protease, ZIKV protease and WNV protease.

19. The method of any one of claims 1-18, wherein the Cas protein is selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

20. The method of claim 19, wherein the Cas protein is catalytically impaired.

21. The method of claim 20, wherein the Cas protein is nCas9 or dCpf1.

22. The method of any one of claims 1-21, wherein the cell is a myeloid cell.

23. The method of any one of claims 1-22, wherein the cell is ex vivo, or in vivo in a human patient.

24. The method of claim 23, wherein the patient suffers from a cancer.

25. One or more polynucleotides encoding a CRISPR-associated (Cas) protein, a nucleobase deaminase, a single-guide RNA (sgRNA), and a helper single-guide RNA (hsgRNA), wherein the sgRNA and the hsgRNA are selected from the sequences from Table 1.

26. A cell prepared by the method of any one of claims 1-24.

27. A human genomic sequence encoding a mutant human CD33, CD123, CD47, CD45 or CLL1, wherein the genomic sequence comprises an edited base introduced by a method of any one of claims 1-24.

28. An mRNA that can be transcribed from the genomic sequence of claim 27.

29. A mutant protein encoded by the genomic sequence of claim 27.

30. A cell comprising the genomic sequence of claim 27, the mRNA of claim 28 or the protein of claim 29.

31. A method of reducing toxicity in a patient undergoing a therapy targeting a cell surface antigen on a cancer cell, comprising administering to the patient the cell of claim 26.

32. A method of reducing toxicity in a patient undergoing a therapy targeting a cell surface antigen on a cancer cell, comprising administering to the patient the polynucleotides of claim 25.
